SEQUENCE LISTING (55 sequences)

SEQ ID NO: 1 - DNA, 528 bp, Arabidopsis thaliana
atgaagctcgaattcattattgttgctatgatgctaagtctcgtactcgtctccggtgagattttaactaaatcctcaccggctccgtcaccggatctagcagattcgcctttaatccacgcatcaccaccatcgaaactcggatctcataattctccagcggaatctccaattgaatactcttctcctccagagcctgaaacagaacactctccatctccttctccggcgaattctccatcggtttctccaccattaccgaatgattctcaatctccttcttcgtctgcttctccgtctccgtcaccggaagctagcgatgtgaatcacagtgatattactgggatcgaaggggagaagttgccgtcgggaagcggtggaggaatgagcggcgggaagaaagttggagttgcttttggagcgattgcggcggtttgtgtggttggagtcgccggatttgtgtacaagaaacggcaagagaatattcgcaggtctcgttacggttacgccgccagagagattctgtaa

SEQ ID NO: 2 - protein, 175 aa, Arabidopsis thaliana
MKLEFIIVAMMLSLVLVSGEILTKSSPAPSPDLADSPLIHASPPSKLGSHNSPAESPIEYSSPPEPETEHSPSPSPANSPSVSPPLPNDSQSPSSSASPSPSPEASDVNHSDITGIEGEKLPSGSGGGMSGGKKVGVAFGAIAAVCVVGVAGFVYKKRQENIRRSRYGYAAREIL

SEQ ID NO: 3 - DNA, 525 bp, Populus trichocarpa
atgggtagcatcactggattagtgcttgttttagccttgtttttattgcaaatctcctcctcctccgcggaaacacctgaacagtcaccatctccatctccatctaccgaagaatccgccgcccccgccaattcaccatttctatctcctcctcttccatctccttcaccagaaactggatctccgtcagattcgccgttggcatccccaccagcaccaccgccttcagatccggttccgtccgttgttccaggctcagcacctgcatcagcgccgacggaagggagcgagatcaatcacagcaacaatgtggaagcaggaagtgggggtgaagggagtgggggcgacggaagtgagggtgaaggagaatcgaagggaatgagcggagggaagaaggcggggatagtagtgggagtgatagtggcggcgtgtatggttggatttggaggattggtttataagaagagacaagataacattcgaaggtctgattatggttatgctgctagaagagagattctctga

SEQ ID NO: 4 - protein, 174 aa, Populus trichocarpa
MGSITGLVLVLALFLLQISSSSAETPEQSPSPSPSTEESAAPANSPFLSPPLPSPSPETGSPSDSPLASPPAPPPSDPVPSVVPGSAPASAPTEGSEINHSNNVEAGSGGEGSGGDGSEGEGESKGMSGGKKAGIVVGVIVAACMVGFGGLVYKKRQDNIRRSDYGYAARREIL

SEQ ID NO: 5 - DNA, 516 bp, Populus trichocarpa
atggctaccaccaccaccattagaagaacaccaattgtttacgctcttttagccttcttcttgttattacgattctcctcctccacggatacacctcctgaacaatcaccatctccatctcctcaacaatccgattcaccattactatctcctcctcctcttcttccacccccctctctctcaccagaaaccggatctccctcaccgacgacaatggcatccccaccagcatcacctccttcagatctgacggctcatgtgcccgcaccggctgagaatgttccagatccggatccgtcggtggcgagcgatataaatgtgaaagcaggaaatgggagtgaagacgatgaagaacaagggtctgagggaatgagtggggggaagaaggcggggatagcagcagcagttataggggcagcttgtttggttggctttggaggattggtttataagaagagacaggataatattcgaaggtctgcttatggttatgctgctagaagagagcttctttga

SEQ ID NO: 6 - protein, 171 aa, Populus trichocarpa
MATTTTIRRTPIVYALLAFFLLLRFSSSTDTPPEQSPSPSPQQSDSPLLSPPPLLPPPSLSPETGSPSPTTMASPPASPPSDLTAHVPAPAENVPDPDPSVASDINVKAGNGSEDDEEQGSEGMSGGKKAGIAAAVIGAACLVGFGGLVYKKRQDNIRRSAYGYAARRELL

SEQ ID NO: 7 - DNA, 629 bp, Arabidopsis thaliana
atggatcgcaatttcctcctaacagttacattgatctgcattgtcgtcgccggtgtcggtggccaatctcctatctcttctccgaccaaatctcccaccactccttctgctccaactacttcccctactaaatcccccgccgttacttctcccactacggctccggcgaaaactccaactgcttcggcttcttcaccggttgagtcaccaaaatctccggctcctgttagcgagtcgtctccaccaccgacacctgttccagagagctctcctccggttcctgcaccaatggtttcttctccagtgagctctccaccggttccagcaccagtagctgattctcctccagctccggtagccgctccggttgctgatgtaccggctcctgctccaagcaagcataagaagactacaaagaaatcgaaaaagcatcaagctgcacctgctccggctccggaacttctcggtccacctgcaccaccgactgaatctcccggacctaactccgacgctttttctcccggtccttccgccgacgatcagagcggagcagcgagcacaagggtgttgaggaatgtagcggtgggagcggttgcaaccgcatgggccgttctcgttatggcattcta

SEQ ID NO: 8 - protein, 209 aa, Arabidopsis thaliana
MDRNFLLTVTLICIVVAGVGGQSPISSPTKSPTTPSAPTTSPTKSPAVTSPTTAPAKTPTASASSPVESPKSPAPVSESSPPPTPVPESSPPVPAPMVSSPVSSPPVPAPVADSPPAPVAAPVADVPAPAPSKHKKTTKKSKKHQAAPAPAPELLGPPAPPTESPGPNSDAFSPGPSADDQSGAASTRVLRNVAVGAVATAWAVLVMAF

SEQ ID NO: 9 - DNA, 1080 bp, Arabidopsis thaliana
atgggtttcattggtaagagtgtattagtgagtctcgtagcactttggtgcttcacttcctctgtcttcactgaagaagtgaaccataagactcaaaccccttctctagctcctgctcctgctccttaccaccacggccaccaccacccacaccctcctcatcaccaccaccctcaccctcaccctcacccccacccaccggccaaatctcctgttaaacccccggttaaggcaccagtgtctccaccagccaaacctccggttaagcccccggtttacccacccaccaaagccccggtgaaacccccaactaaacccccggtcaagccacctgtttccccaccagccaaacctccggttaagccaccagtttaccctccaaccaaagctccagttaagcccccaactaaacccccagtcaagccaccagtttaccctccaaccaaagccccggttaagcccccaactaaacccccagtcaagccaccagtttaccctccaaccaaagccccggttaagcccccaactaaacccccggtcaagccaccagtttccccaccagccaaacctccagttaagcccccggtttaccctcctaccaaagctccggttaagcctccggtttctcctccgacgaagccacctgttacaccgccggtttacccgcctaagttcaaccgtagtctagtcgccgttcggggtacggtttactgcaaaagctgcaagtacgctgctttcaacacactcttaggcgctaaacccatcgaaggtgctacggtgaaactagtgtgtaagagcaagaagaacataacagcggagacgacgacagacaagaacggatacttcttgttgttggctcctaagacggtgacgaacttcggtttcagaggatgtcgtgtgtatttggtgaaatcaaaggactacaaatgcagcaaagtctcaaagctcttcggtggagatgtcggcgctgaacttaagcctgagaaaaagctgggaaaatctacggtggtcgtcaacaagctcgtatacggtctctttaacgttggtccatttgcctttaacccgtcttgccccaaatga

SEQ ID NO: 10 - protein, 359 aa, Arabidopsis thaliana
MGFIGKSVLVSLVALWCFTSSVFTEEVNHKTQTPSLAPAPAPYHHGHHHPHPPHHHHPHPHPHPHPPAKSPVKPPVKAPVSPPAKPPVKPPVYPPTKAPVKPPTKPPVKPPVSPPAKPPVKPPVYPPTKAPVKPPTKPPVKPPVYPPTKAPVKPPTKPPVKPPVYPPTKAPVKPPTKPPVKPPVSPPAKPPVKPPVYPPTKAPVKPPVSPPTKPPVTPPVYPPKFNRSLVAVRGTVYCKSCKYAAFNTLLGAKPIEGATVKLVCKSKKNITAETTTDKNGYFLLLAPKTVTNFGFRGCRVYLVKSKDYKCSKVSKLFGGDVGAELKPEKKLGKSTVVVNKLVYGLFNVGPFAFNPSCPK

SEQ ID NO: 11 - DNA, 636 bp, Oryza sativa
atggcagcggtagcacgagcactagtcctcggcgccgtcgtcgtctgcgccgccgtcgtgatggctgtcaccgccgcggcggacggcgaggcggcggcggcggtcgtcgtcggcttggccaagtgcggtggctgctccaggaagaacatgaaggcgcaggatgctttcaagggtctccaggtggcgatcaagtgcaggaacggcgacggcgagtacgagagcaaggccgtcggcgacctcgacggcgacggcgccttcagcgtgcccctcgccgccgacgacctccacggcgccgccgactgctttgcgcagctgcacagcgcggagagcagcacgccgtgccccgggcaggagccgtccaagatcgtgccgctgtcgtcaacaacggacaacggcggtgataaggctaacaccttcgtcgccgtcgccggcaagaggatgcgctactcgtcgtcggcggagtgcaactcggcgttcctgtgccccttcttcgactacttccacaagagaccccaaggccccaagccgacgccgttgccgaagccgacgccggcgaacggtggtggcgcggcgaacggtggcggagctgcggcgccgtctcccagccctccggccggcatatcacagctgaattaa

SEQ ID NO: 12 - protein, 211 aa, Oryza sativa
MAAVARALVLGAVVVCAAVVMAVTAAADGEAAAAVVVGLAKCGGCSRKNMKAQDAFKGLQVAIKCRNGDGEYESKAVGDLDGDGAFSVPLAADDLHGAADCFAQLHSAESSTPCPGQEPSKIVPLSSTTDNGGDKANTFVAVAGKRMRYSSSAECNSAFLCPFFDYFHKRPQGPKPTPLPKPTPANGGGAANGGGAAAPSPSPPAGISQLN

SEQ ID NO: 13 - DNA, 2869 bp, Arabidopsis thaliana
atgacccgaagaacaatggagaagccttttggctgcttccttctcctcttttgtttcaccatctccatttttttctattccgcagcggctctcaccgatgggaagcttcttttcttacaagacgccaacttttggcattatcagaaaacggagatcttcccgacgacatcgagtacgaagtcgatttagacctcaaattgctaataacagacttaaaagggcttacattgcccttcaagcttggaaaaaggcattctactctgatccgttcaacactgcggctaattgggttggtccagacgtgtgttcttacaaaggagttttctgtgcaccagcacttgacgacccaagcgtattggttgtggcggggattgaccttaaccatgcggatatagccggatacttgccgcctgagctaggtttattgaccgatgtcgccttgtttcacgttaattccaacaggttttgcggagttattccgaaaagtctttcgaagctaacgttaatgtatgaattcgacgtcagcaataaccgtttcgttggtccgtttccgacagttgctctttcttggccgtctttgaaattccttgacataagatacaatgattttgaagggaaactaccaccagagatctttgataaagatctcgacgccatcttcttgaacaacaatagatttgagtcgaccatccccgagacaattggtaaatcaacagcttccgttgtaaccttcgcacacaataaatttagcggttgcatcccaaaaaccattggccagatgaagaacctcaacgagattgtgtttattgggaacaacctaagtggttgcttaccgaacgaaatcggttcgttgaacaatgtgactgtgtttgatgctagcagcaatgggttcgtcggaagtttgccatcgaccttgtcgggcttagccaacgtggaacaaatggatttctcgtacaataagttcaccggatttgtcacagacaatatctgcaagttgcccaaattgtcaaacttcacattttcttacaatttcttcaatggagaagctcaaagttgtgtgcctggatcgagccaagagaaacagtttgatgacacgagcaattgcttacagaacagaccaaaccagaagtcggctaaagaatgtttgccggtggttagccgtcctgtggattgcagtaaagacaagtgtgccggtggaggtggcggtggttcaaacccatcaccaaaaccaacaccaacaccaaaggcaccggagcctaagaaagaaataaacccaccaaacttagaagagccatctaagccaaaaccagaagaatctcccaaaccacaacagcctagtccaaagcctgagacaccatcacatgagccttctaacccaaaggaacctaagccagagtcaccaaaacaggaatctccaaaaacggaacagccaaaacctaagcctgagtcaccaaaacaagagtcgcctaagcaagaggcaccaaaaccagaacagccaaaacctaagcctgagtcaccaaaacaagagtcgtctaagcaagagccaccaaaaccagaagaatctcctaaaccggaacctccaaaaccagaggagtcacctaagcctcaaccaccaaagcaagagacaccaaaaccagaggagtcacctaagcctcaaccaccaaagcaagagacaccaaaaccagaggagtcacctaagcctcaaccaccaaagcaagagaccccaaaaccagaggagtcacctaagcctcaaccaccaaagcaagaacaaccaccaaagacagaggcaccaaaaatggggtcgcctccattggaatcaccggtaccaaatgatccttacgatgcatctccgattaaaaaacgtcgtcctcaaccaccatcaccctcgacagaagagacaaagacaacgtcgccacaatctcctccagtacactctccaccaccaccaccaccggttcattccccaccgccgcctgtattttcacctcctcctccgatgcattctcctccaccaccggtttactcaccaccacctccagtacactctcctccaccaccaccagtccactctcctccaccaccagtacattcaccaccaccaccagtccattctcctcctccaccggtccattcaccaccaccaccagtccactctcctcctccaccagtccactctcctcctccaccggtccagtcacctccaccaccaccagttttctctccaccaccgcctgcaccaatctactcacctcctcctccaccagtccactccccgcctccaccggtccactctccaccaccgccaccagtccactcaccacctcctcctgttcactcaccgcctcctccagttcactccccgccgcctccagtccattctccaccaccaccagtccactctccaccacctccgtcacctatctactctcctcctcctccagttttctcaccgccaccgaaacctgtaacgcctctaccaccagcaacatcgccaatggcaaacgctccaacaccttcatcatcagaatcaggagaaatctcaacaccggttcaagctccgaccccagactctgaggacatcgaagcaccatcagattcaaatcattcaccagtgttcaaatcaagtccagctccatcaccagattccgagccagaagttgaagcaccagtaccatcaagcgaaccagaagttgaagcaccaaaacaatcagaagccaccccttcgtcatcaccgccatcttcaaatccatcaccagatgtaactgcaccaccatcggaagacaacgacgacggcgataatttcatcctcccaccaaacattggacaccaatatgcatcgccaccaccaccgatgttcccaggctactga

SEQ ID NO: 14 - protein, 956 aa, Arabidopsis thaliana
MTRRTMEKPFGCFLLLFCFTISIFFYSAAALTDEEASFLTRRQLLALSENGDLPDDIEYEVDLDLKFANNRLKRAYIALQAWKKAFYSDPFNTAANWVGPDVCSYKGVFCAPALDDPSVLVVAGIDLNHADIAGYLPPELGLLTDVALFHVNSNRFCGVIPKSLSKLTLMYEFDVSNNRFVGPFPTVALSWPSLKFLDIRYNDFEGKLPPEIFDKDLDAIFLNNNRFESTIPETIGKSTASVVTFAHNKFSGCIPKTIGQMKNLNEIVFIGNNLSGCLPNEIGSLNNVTVFDASSNGFVGSLPSTLSGLANVEQMDFSYNKFTGFVTDNICKLPKLSNFTFSYNFFNGEAQSCVPGSSQEKQFDDTSNCLQNRPNQKSAKECLPVVSRPVDCSKDKCAGGGGGGSNPSPKPTPTPKAPEPKKEINPPNLEEPSKPKPEESPKPQQPSPKPETPSHEPSNPKEPKPESPKQESPKTEQPKPKPESPKQESPKQEAPKPEQPKPKPESPKQESSKQEPPKPEESPKPEPPKPEESPKPQPPKQETPKPEESPKPQPPKQETPKPEESPKPQPPKQETPKPEESPKPQPPKQEQPPKTEAPKMGSPPLESPVPNDPYDASPIKKRRPQPPSPSTEETKTTSPQSPPVHSPPPPPPVHSPPPPVFSPPPPMHSPPPPVYSPPPPVHSPPPPPVHSPPPPVHSPPPPVHSPPPPVHSPPPPVHSPPPPVHSPPPPVQSPPPPPVFSPPPPAPIYSPPPPPVHSPPPPVHSPPPPPVHSPPPPVHSPPPPVHSPPPPVHSPPPPVHSPPPPSPIYSPPPPVFSPPPKPVTPLPPATSPMANAPTPSSSESGEISTPVQAPTPDSEDIEAPSDSNHSPVFKSSPAPSPDSEPEVEAPVPSSEPEVEAPKQSEATPSSSPPSSNPSPDVTAPPSEDNDDGDNFILPPNIGHQYASPPPPMFPGY

SEQ ID NO: 15 - DNA, 2647 bp, Oryza sativa
atggctatgaagagagtggtgtttctgttgctgcttgtggcggcatctgcattggtgaagtcgtcaagaggaggaggaggaggggaggagaagttggggaagttctatggatggcggcgccatctttcttctggaccagctgcctcttcgttggttctcagtggggatctcgtagacaaaatatggtcagtttgcttacaagacatagttagcccagaggatacatttggctttggggaatcatttgcctgggatgagttatcgagtcattccacggaagatgaactgaaggcaacactgttcatggaacttatggctcttcttccacctgagaaatcttcttttacttatgattgtatccgtgcaaactgcttcagcctgggtgttccacaaatattcagtgttgcactgagcaactatcttgagagccagaaatcattggtcggttcaaacttctatccgagacggcgtttggtagataaactgatcggagatgctccttccatggctccagctttcgcaccatccatgtcctctggcggtgaagttcactctcccctctctgtggcggaggcaccacttacaccttccaactctctgaacatggaacctcccagcccatattatcccagtaaatctgcacacaagcatcagggagtggcacctccagtttcaccttcagaggagtatcatgactacatgaaagtagtcttgatcgctgttcttccaacagccgcactctcattcctagctgcatttctatgtttctactgctgtggatgcaacaaaagcaaggtctcggttggcgagcaacgagatgaccatcctcttcttcacttgcagttctctaacttgcctggttcgtcacctgatgtgcatgttcctgccagcccgcttcataaggatgatcatggggttaggccatccaatgctggtgtaagcatgagcaagtgctttccatgttgttttaaaacctcaagtgatgccacaacacctacactagtaactggaggaacacaggagaacaatgccacaagtgatgcccctaaactgatgcctccgccacccccacctcctccacctccacctccacctccccctccacctcctccgcgtccacctcctccgcctccacctatcaagaagggtgctccaccacctgcacctcctaaagccaccatggcaagatttcctaagctgtcacctacagagtcaagtcgttctgaagagtcatctgcaagcgagctggccagtgaatcgtctgaaactgaggtgaatgctccaagagccaagctccggcctttctattgggataaagttcttgctaatcctgatcagtcaatggcctggcatgacatcaaatttggttcttttcatgtgaacgaggagatgatagaagaattgtttggttatggtgctggcaaccaaaacaatgtcaaggacaaggaaatttccattgcagatccttcacctcagcatgtttctcttcttgatgttaagaaatcatgcaatctggcagttgtttttaaggcaatgaatgtgagggcagaggaaattcatgatgctctggttgaagggaatgaacttcctagattacttcttgagacaatcttgaggatgaaaccgactgatgaggaggaacagaaactcaggctttataatggggactgctcgcagctaggcctagcagaacaagtgatgaaggcactaattgatattccttttgctttcgagaggattagagctttgcttttcatgtcctctttgcaggaagatgcttcaagtctaagggaatcattcctccaattggaggctgcttgcggtgaactcaagcaccgccctttttctgaaactactagaagccattctcaaaactggaaatcgtttgaatgatgggacgttccgtggtggtgctaacgcgttcaaacttgacactcttcttaagttatcagatgtcaagggagctgatgggaagaccacattgctgcactttgttgttcaagagatcattcgatctgaaggtgttcgcgaagcaagactcgccatggaaaatggaagaagtccaccatttcctagtacttcggatgacaattccaatgaatctctacaggaggatggcaattactactccaaccttggccttaagattgtatcagggcttagtaatgagctggacaatgtcaagagagtagcagcactggatgctgatgctttgtctacaagtgtggcaaatctgcgacacgaattattgagagccaaagaattcctgaactccgacatggcatcgctagaagagaacagtggattccaccgctcgttagaaagtttcatagaacatgcagagactgagaccaattttctcctgaaagaagacaagagattgaggatgttagtgaagagaacgattcgatatttccatggaaatgatgagaaagatgacggttttcgcctgttcgtcattgtaagagattttctggtgatgctagataaggcatgcaaggaggttggtgcttcacaaaagaaggcaacaaataaatctcaagccaatggcaactctaataacccatcatcccagtcaaatcctcaggagcaacagtttcctgctgttttggatcatcattttgatagctcagactctaatgattga

SEQ ID NO: 16 - protein, 881 aa, Oryza sativa
MAMKRVVFLLLLVAASALVKSSRGGGGGEEKLGKFYGWRRHLSSGPAASSLVLSGDLVDKIWSVCLQDIVSPEDTFGFGESFAWDELSSHSTEDELKATLFMELMALLPPEKSSFTYDCIRANCFSLGVPQIFSVALSNYLESQKSLVGSNFYPRRRLVDKLIGDAPSMAPAFAPSMSSGGEVHSPLSVAEAPLTPSNSLNMEPPSPYYPSKSAHKHQGVAPPVSPSEEYHDYMKVVLIAVLPTAALSFLAAFLCFYCCGCNKSKVSVGEQRDDHPLLHLQFSNLPGSSPDVHVPASPLHKDDHGVRPSNAGVSMSKCFPCCFKTSSDATTPTLVTGGTQENNATSDAPKLMPPPPPPPPPPPPPPPPPPPRPPPPPPPIKKGAPPPAPPKATMARFPKLSPTESSRSEESSASELASESSETEVNAPRAKLRPFYWDKVLANPDQSMAWHDIKFGSFHVNEEMIEELFGYGAGNQNNVKDKEISIADPSPQHVSLLDVKKSCNLAVVFKAMNVRAEEIHDALVEGNELPRLLLETILRMKPTDEEEQKLRLYNGDCSQLGLAEQVMKALIDIPFAFERIRALLFMSSLQEDASSLRESFLQLEAACGELKHRLFLKLLEAILKTGNRLNDGTFRGGANAFKLDTLLKLSDVKGADGKTTLLHFVVQEIIRSEGVREARLAMENGRSPPFPSTSDDNSNESLQEDGNYYSNLGLKIVSGLSNELDNVKRVAALDADALSTSVANLRHELLRAKEFLNSDMASLEENSGFHRSLESFIEHAETETNFLLKEDKRLRMLVKRTIRYFHGNDEKDDGFRLFVIVRDFLVMLDKACKEVGASQKKATNKSQANGNSNNPSSQSNPQEQQFPAVLDHHFDSSDSND

SEQ ID NO: 17 - DNA, 22 bp, artificial
  synthetic oligonucleotide primer
gatgctaagtctcgtactcgtc

SEQ ID NO: 18 - DNA, 21 bp, artificial
  synthetic oligonucleotide primer
ctcttgccgtttcttgtacac

SEQ ID NO: 19 - DNA, 20 bp, artificial
  synthetic oligonucleotide primer
atcctccgtcttgaccttgc

SEQ ID NO: 20 - DNA, 20 bp, artificial
  synthetic oligonucleotide primer
gacctgcctcatcatactcg

SEQ ID NO: 21 - DNA, 20 bp, artificial
  synthetic oligonucleotide primer
tcgccggatttgtgtacaag

SEQ ID NO: 22 - DNA, 20 bp, artificial
  synthetic oligonucleotide primer
aatctctctggcggcgtaac

SEQ ID NO: 23 - DNA, 23 bp, artificial
  synthetic oligonucleotide primer
ggtaacattgtgctcagtggtgg

SEQ ID NO: 24 - DNA, 23 bp, artificial
  synthetic oligonucleotide primer
aacgaccttaatcttcatgctgc

SEQ ID NO: 25 - DNA, 21 bp, artificial
  synthetic oligonucleotide primer
caagtgtttcgcacctttctc

SEQ ID NO: 26 - DNA, 27 bp, artificial
  synthetic oligonucleotide primer
ttcatatcaaacaatatttttgaactc

SEQ ID NO: 27 - DNA, 21 bp, artificial
  synthetic oligonucleotide primer
ggttcggatatctttcggttc

SEQ ID NO: 28 - DNA, 20 bp, artificial
  synthetic oligonucleotide primer
gcaactccaactttcttccc

SEQ ID NO: 29 - DNA, 19 bp, artificial
  synthetic oligonucleotide primer
attttgccgatttcggaac

SEQ ID NO: 30 - DNA, 22 bp, artificial
  synthetic oligonucleotide primer
gatgctaagtctcgtactcgtc

SEQ ID NO: 31 - DNA, 21 bp, artificial
  synthetic oligonucleotide primer
ctcttgccgtttcttgtacac

SEQ ID NO: 32 - DNA, 20 bp, artificial
  synthetic oligonucleotide primer
tcgccggatttgtgtacaag

SEQ ID NO: 33 - DNA, 20 bp, artificial
  synthetic oligonucleotide primer
aatctctctggcggcgtaac

SEQ ID NO: 34 - DNA, 23 bp, artificial
  synthetic oligonucleotide primer
ggtaacattgtgctcagtggtgg

SEQ ID NO: 35 - DNA, 23 bp, artificial
  synthetic oligonucleotide primer
aacgaccttaatcttcatgctgc

SEQ ID NO: 36 - protein, 20 aa, artificial
  N-terminal amino acid sequence for YS1
EILTKSSXAXSXDLADSPLI

SEQ ID NO: 37 - protein, 19 aa, artificial
  N-terminal amino acid sequence for YS2
ILTKSSXAXSXDLADSPLI

SEQ ID NO: 38 - DNA, 21 bp, artificial
  synthetic oligonucleotide primer
taccaaaaaccaagctccatg

SEQ ID NO: 39 - DNA, 21 bp, artificial
  synthetic oligonucleotide primer
agaacacacgtctggaccaac

SEQ ID NO: 40 - DNA, 21 bp, artificial
  synthetic oligonucleotide primer
ggacggtagtatcggctgatc

SEQ ID NO: 41 - DNA, 21 bp, artificial
  synthetic oligonucleotide primer
gacttttcggaataactccgc

SEQ ID NO: 42 - DNA, 21 bp, artificial
  synthetic oligonucleotide primer
ggacggtagtatcggctgatc

SEQ ID NO: 43 - DNA, 21 bp, artificial
  synthetic oligonucleotide primer
gacttttcggaataactccgc

SEQ ID NO: 44 - DNA, 21 bp, artificial
  synthetic oligonucleotide primer
aggcttaggtgactcctctgg

SEQ ID NO: 45 - DNA, 21 bp, artificial
  synthetic oligonucleotide primer
ttggtaaatcaacagcttccg

SEQ ID NO: 46 - DNA, 21 bp, artificial
  synthetic oligonucleotide primer
aaagagttcaaaacgcaaacg

SEQ ID NO: 47 - DNA, 21 bp, artificial
  synthetic oligonucleotide primer
tgatttaccaattgtctcggg

SEQ ID NO: 48 - DNA, 37 bp, artificial
  synthetic oligonucleotide primer
aaattggatccatgaagctcgaattcattattgttgc

SEQ ID NO: 49 - DNA, 38 bp, artificial
  synthetic oligonucleotide primer
aaattgagctcttacagaatctctctggcggcgtaacc

SEQ ID NO: 50 - DNA, 20 bp, artificial
  synthetic oligonucleotide primer
ccttcgcaagacccttcctc

SEQ ID NO: 51 - protein, 22 aa, artificial
  N-terminal amino acid sequence from AtAGP57C
SSXAXSXDLADSXLIHASXXSK

SEQ ID NO: 52 - protein, 4 aa, artificial
  characteristic extensin repeat having hydroxyproline residues for attachment sites
SXXX

SEQ ID NO: 53 - protein, 5 aa, artificial
  characteristic extensin repeat having hydroxyproline residues for attachment sites
SXXXX

SEQ ID NO: 54 - protein, 6 aa, artificial
  characteristic extensin repeat having hydroxyproline residues for attachment sites
SXXXXX

SEQ ID NO: 55 - protein, 5 aa, artificial
  characteristic pentapeptide found in proline- rich proteins
PXVYK

(19) United States
(12) Patent Application Publication
(10) Pub. No.: US 2014/0363868 A1
(43) Pub. Date: Dec. 11, 2014

(54) TRANSGENIC PLANTS AND METHODS OF USING SAME

(71) Applicant: UNIVERSITY OF GEORGIA RESEARCH FOUNDATION, INC., Athens, GA (US)

(72) Inventors: Debra Mohnen, Athens, GA (US); Li Tan, Athens, GA (US)

(73) Assignee: UNIVERSITY OF GEORGIA RESEARCH FOUNDATION, INC., Athens, GA (US)

(21) Appl. No.: 14/373,946

(22) PCT Filed: Jan. 25, 2013
(86) PCT No.: PCT/US13/23221

§ 371 Date: Jul. 23, 2014

Related U.S. Application Data
(60) Provisional application No. 61/591,117, filed on Jan. 26, 2012.

(51) Int. Cl. C12N 15/82; C12P 7/06

(52) U.S. Cl. CPC C12N 15/8251; C12P 7/06; C12N 15/8245
USPC 435/161; 800/298; 435/419

(57) ABSTRACT

Provided herein are plants having altered expression of a coding region encoding a hydroxyproline-rich glycoprotein. In some embodiments the expression is decreased, and the resulting plant has decreased recalcitrance, increased growth, or a combination thereof. In some embodiments the expression is increased, and the resulting plant has decreased growth, biomass that is more cross-linked, biomass that is more dense, or a combination thereof. Also provided are methods for making and using such plants.

### CROSS-REFERENCE TO RELATED APPLICATIONS

This application claims the benefit of U.S. Provisional Application Ser. No. 61/591,117 filed Jan. 26, 2012, which is incorporated by reference herein.

### GOVERNMENT FUNDING

The present invention was made with government support under Grant Nos. 0646109 and DBI-0421683, awarded by the NSF, and Grant Nos. DE-PS02-06ER64304, DE-FG02-09ER20097, and DE-AC05-000R22725, awarded by the DOE. The Government has certain rights in this invention.

### BACKGROUND

The plant primary cell wall is a matrix of the polysaccharides cellulose, hemicellulose, and pectin with small amounts (˜10%) of enzymes and structural proteins such as the hydroxyproline (Hyp)-rich glycoproteins (HRGPs) including extensins and arabinogalactan proteins (AGPs) (Burton et al., 2010, Nature Chem. Biol., 6:724-732). The prevailing tethered network model of the cell wall depicts the type I primary cell wall as a xyloglucan tethered cellulose network embedded in an independent pectin gel (reviewed in Albersheim et al., 2011, Plant cell walls. (New York: Garland Science, Taylor & Francis Group). pp 227-272) while Type II primary wall models consist of a xylan tethered cellulose network and a reduced pectin component. Cell wall components are proposed to interact both covalently and non-covalently to form the functional wall. Non-covalent interactions include ionic bonds between pectic homogalacturonan (HG) domains (Caffall and Mohnen, 2009, Carbohydr. Res. 344:1879-1900) and hydrogen-bonds between cellulose chains and between cellulose and regions of xyloglucan or xylan (Pauly et al., Plant J. 20:629-639). In addition to non-covalent interactions, covalent bonds appear to cross-link at least some of the wall polymers into a durable and rigid structure that can withstand extreme turgor pressures. The full extent of these covalent cross-links is not known, but they include tyrosine-based linkages between the family of wall HRGPs known as extensins (Fry, 1982, Biochem J. 204:449-455; Held et al., 2004, J. Biol. Chem. 279:55474-55482); borate esters between pectin rhamnogalacturonan (RG)-II monomers (Ishii et al., 1999, J. Biol. Chem. 274:13098-13104); and ester linkages between polysaccharides and phenolic moieties of lignin residues (Ishii, 1997, Plant Sci. 127: 111-127; Ishii and Hiroi, 1990, Carbohydr. Res. 206:297-310; Ishii and Hiroi, 1990, Carbohydr. Res. 196:175-183; Harris and Trethewey, 2010, Phytochem. Rev. 9:19-33).

Current models of xylan and pectin structure portray independent wall polysaccharides. Xylan is a major hemicellulosic polysaccharide in secondary walls and in grass primary walls, and is also present in reduced amounts (˜5%) in dicot primary walls (Darvill et al., 1980, Plant Physiol. 66:1135-1139). Xylan is composed of a backbone of 1→4-linked β-D-xylopyranosyl residues that may be partially glycosylated at O-2 or O-3 with arabinofuranosyl and/or with 4-O-methyl glucuronosyl residues at O-2 to form arabinoxylan and/or glucuronoarabinoxylan (reviewed in Doering et al., 2012, Mol. Plant. 5:769-771; Kulkami et al., 2012, Glycobiology. 22:439-451). Arabinoxylan in grasses is commonly arabinosylated at Xyl O-3 and may, to a lesser degree, include O-2 and O-3 di-arabinosyl substitution. Dicot xylan is less frequently arabinosylated, with reported arabinosylation generally at the O-2 of Xyl (reviewed in Scheller and Ulvskov 2010, Annu Rev. Plant. Biol. 61:263-289). Xylan may also be acetylated at O-3.

Pectin is a family of galacturonic acid (GalA)-rich polysaccharides that accounts for 30-35% (w/w) of primary cell walls in dicots and non-graminaceous monocots, but is also present in secondary walls and in grasses. The most abundant pectic polysaccharide, homogalacturonan (HG), is a linear homopolymer of 1,4-linked α-D-GaJA residues that accounts for ˜65% of pectin. HG may reach lengths of 100 residues or more (Thibault et al., 1993, Carbohydr. Res., 238:271-286). The other major pectin, rhamnogalacturonan I (RG-I), comprises 20-35% of pectin and a repetitive [-2-α-L-Rhap-1→4-α-D-GalAp-1-] disaccharide backbone with 20-80% of the rhamonsyl residues decorated by sidechains of 1,5-arabinans, 1,4-galactans, and type I and type II arabinogalactans (Mohnen, 2008, Curr. Opin. Plant Biol., 11:266-277). The GalA residues in HG may also be substituted with four complex side chains to form RG-II, representing ˜10% of wall pectin, or to a lesser degree with terminal-xylosyl or apiosyl residues (Mohnen, 2008, Curr. Opin. Plant Biol., 11:266-277; Harholt et al., 2010, Plant Physiol., 153:384-395). Acetylation and methylation of pectin in vivo may change the charge and hydrophobicity of the polysaccharides, and hence, its roles in the plant.

The arabinogalactan proteins (AGPs) are highly glycosylated hydroxyproline-rich glycoproteins (HRGPs) that have of up to 95% carbohydrate. Clustered non-contiguous Hyp residues in the AGP protein backbone usually have covalently attached so-called type II arabinogalactan (AG) polysaccharides. Individual AG glycans in an AGP have up to 150 sugar residues and are rich in arabinose (Ara) and galactose (Gal) (Kieliszewski, 2001, Phytochemistry 57:319-323; Showalter, 2001, Cell. Mol. Life. Sci., 58:1399-1417). An individual AG glycan consists of a β-1,3-galactan backbone with β-1,6-galactosyl branches that are decorated with arabinosyl residues and occasionally with minor sugar residues such as glucuronic acid (GlcA), rhamnose (Rha), and fucose (Fuc) (Ellis et al., 2010, Plant Physiol., 153:403-419; Carpita and Gibeaut, 1993, Plant J. 3:1-30; Tan et al., 2010, J. Biol. Chem. 285:24575-24583). However, as noted above, type II AGs are also found as side chains of the pectin RG-I (Caffall and Mohnen, 2009, Carbohydr. Res. 344:1879-1900) and as free polysaccharides (Ponder and Richards, 1997, Carbohydr. Polym. 34:251-261).

### SUMMARY OF THE INVENTION

Described herein is a new proteoglycan-based plant cell wall structure in which cell wall pectin and hemicellulose glycans are covalently linked to structural proteins such as arabinogalactan protein (AGP), extensin, extensin-like proteins or other plant hydroxyproline-rich glycoproteins (HRGPs). This is in contrast to the current view of plant walls as complex interacting networks of separate cellulose, hemicellulosic, and pectic polysaccharides. The research described herein has identified a new complex proteoglycan from Arabidopsis cell walls, APAP1 (Arabinoxylan-Pectin-Arabinogalactan Protein1), which, in some embodiments, has pectin and arabinoxylan glycan modules covalently linked to the arabinogalactan protein AGP57C (encoded by Arabidopsis gene At3g45230). Two HRGPs, pollen-specific leucine-rich repeat extensin-like protein 1 (PEX1, gene At3g19020) and AtAGP31 (gene At1g28290), have been identified from an Arabidopsis xyloglucan preparations. Also found were at least two proline/hydroxyproline rich protein sequences, a putative proline-rich protein (OSJNBa0031A07.9, gene Os10g0149900) and a formin-like protein (FH10, gene Os02g0161100) from a cellulose prep (containing mainly cellulose) isolated from rice biomass. It is possible that APAP1 is only the first plant wall proteoglycan structure and that additional hydroxyproline-rich glycoprotein cores with unique glycan modules exist in plant cell walls. The evidence suggests a complex proteoglycan network in plant cell walls, a novel concept for wall structure.

The identification of APAP1 and the new model is helpful in understanding cell wall synthesis, structure, and deconstruction, which are major needs for future biofuel and biomaterial production. Most current efforts to decrease biomass recalcitrance focus on either the identification of mutants/natural variants of cell wall polysaccharide biosynthetic genes, or the characterization of polysaccharide carbohydrate-degrading enzymes. None of these efforts focus on hydroxyproline-rich glycoproteins such as arabinogalactan-proteins as the “root” of wall structure. Based on this new concept, it is proposed that manipulation of AGPs and other hydroxyproline-rich glycoproteins will produce more degradable plant biomass for biofuel/material production. In one embodiment, this invention allows the prediction of novel genes and gene combinations, such as glycan domain biosynthetic genes, and novel mutants and variants of value for economical biofuel, biomaterial, and agricultural production.

Provided are methods for using the plants and plant material disclosed herein. In one embodiment, a method includes processing a part of a transgenic plant to result in pulp, wherein the transgenic plant includes decreased expression of a coding region encoding a hydroxyproline-rich glycoprotein compared to a control plant. The processing may include a physical pretreatment, a chemical pretreatment, or a combination thereof. The method may further include hydrolyzing the processed pulp.

In one embodiment, a method includes hydrolyzing a pulp, wherein the pulp includes plant material from a transgenic plant, wherein the transgenic plant includes a mutation in a coding region encoding a hydroxyproline-rich glycoprotein.

In one embodiment, a method includes producing a metabolic product. Such a method may include contacting under conditions suitable for the production of a metabolic product a microbe with a composition that includes a pulp obtained from a part of a transgenic plant, where the transgenic plant includes decreased expression of a coding region encoding a hydroxyproline-rich glycoprotein compared to a control plant.

Methods for using the plants and plant material disclosed herein may further include contacting the pulp with an ethanologenic microbe, such as a eukaryote. The method may further include obtaining a metabolic product, and in one embodiment, the metabolic product includes ethanol. In one embodiment, the hydroxyproline-rich glycoprotein may be an arabinogalactan-protein, a leucine-rich repeat extensin-like polypeptide, a proline-rich polypeptide, an extensin-like polypeptide, or a formin-like polypeptide. In one embodiment, the transgenic plant may be a dicot plant or a monocot plant. In one embodiment, the transgenic plant is a woody plant. In one embodiment, the transgenic plant is a member of the genus Populus. Also included is the processed pulp.

Also provided herein is a transgenic plant that includes altered expression of a coding region encoding a hydroxyproline-rich glycoprotein compared to a control plant. In one embodiment, the alteration in expression may be a decrease in expression. In one embodiment, the alteration in expression may be an increase in expression. In one embodiment, the transgenic plant is not Arabidopsis thaliana. In one embodiment, the hydroxyproline-rich glycoprotein is an arabinogalactan-protein, a leucine-rich repeat extensin-like polypeptide, a proline-rich polypeptide, an extensin-like polypeptide, or a formin-like polypeptide. In one embodiment, the transgenic plant includes a phenotype selected from decreased recalcitrance, increased growth, or the combination thereof. In one embodiment, the transgenic plant includes a phenotype selected from shorter stems, smaller leaves, smaller inflorescences, smaller pollen, shorter roots, reduced total biomass, or a combination thereof. In one embodiment, the transgenic plant is a dicot or a monocot plant. Also provided is a part of the transgenic plant. In one embodiment, the part is chosen from a leaf, a stem, a flower, an ovary, a fruit, a seed, and a callus. Also provided is progeny of the transgenic plant, including a hybrid plant; a wood obtained from the transgenic plant; and a wood pulp obtained from the transgenic plant.

Provided herein is a method for generating a transgenic plant having decreased recalcitrance, increased growth, or the combination thereof, compared to a plant of substantially the same genetic background grown under the same conditions. The method may include transforming a plant cell with a polynucleotide to result in a recombinant plant cell, and generating a transgenic plant from the recombinant plant cell, wherein the transgenic plant has decreased expression of a coding region encoding a hydroxyproline-rich glycoprotein compared to a control plant. The transgenic plant may be a dicot plant or a monocot plant. The method may further include breeding the transgenic plant with a second plant, wherein the second plant is transgenic or nontransgenic. In one embodiment, the increased growth is selected from increased stem growth, increased root growth, or the combination thereof. In one embodiment, the transgenic plant is a woody plant, such as a member of the genus Populus. In one embodiment, the method may further include screening the transgenic plant for decreased recalcitrance, increased growth, or the combination thereof.

Also provided herein is a method for generating a transgenic plant decreased growth compared to a plant of substantially the same genetic background grown under the same conditions. The method may include transforming a plant cell with a polynucleotide to result in a recombinant plant cell, and generating a transgenic plant from the recombinant plant cell, wherein the transgenic plant has increased expression of a coding region encoding a hydroxyproline-rich glycoprotein compared to a control plant. The transgenic plant may be a dicot plant or a monocot plant. The method may further include breeding the transgenic plant with a second plant, wherein the second plant is transgenic or nontransgenic. In one embodiment, the decreased growth is selected from shorter stems, smaller leaves, smaller inflorescences, smaller pollen, shorter roots, reduced total biomass. In one embodiment, the transgenic plant is a woody plant, such as a member of the genus Populus. In one embodiment, the method may further include screening the transgenic plant for decreased recalcitrance, increased growth, or the combination thereof.

As used herein, the term “transgenic plant” refers to a plant that has been transformed to contain at least one modification to result in altered expression of a coding region. For example, a coding region in a plant may be modified to include a mutation to reduce transcription of the coding region or reduce activity of a polypeptide encoded by the coding region. Alternatively, a plant may be transformed to include a polynucleotide that interferes with expression of a coding region. For example, a plant may be modified to express an antisense RNA or a double stranded RNA that silences or reduces expression of a coding region by decreasing translation of an mRNA encoded by the coding region. In some embodiments more than one coding region may be affected. In another embodiment, a coding region in a plant may be modified to increase transcription of the coding region or increase activity of a polypeptide encoded by the coding region. The term “transgenic plant” includes whole plants, plant parts (stems, roots, leaves, fruit, etc.) or organs, plant cells, seeds, and progeny of same. A transformed plant of the current invention can be a direct transfectant, meaning that the DNA construct was introduced directly into the plant, such as through Agrobacterium, or the plant can be the progeny of a transfected plant. The second or subsequent generation plant can be produced by sexual reproduction, i.e., fertilization. Furthermore, the plant can be a gametophyte (haploid stage) or a sporophyte (diploid stage). A transgenic plant may have a phenotype that is different from a plant that has not been transformed.

As used herein, the term “control plant” refers to a plant that is the same species as a transgenic plant, but has not been transformed with the same polynucleotide used to make the transgenic plant.

As used herein, the term “plant tissue” encompasses any portion of a plant, including plant cells, seed mucilage and root mucilage. Plant cells include suspension cultures, callus, embryos, meristematic regions, callus tissue, leaves, roots, shoots, gametophytes, sporophytes, pollen, seeds, and microspores. Plant tissues can be grown in liquid or solid culture, or in soil or suitable media in pots, greenhouses, or fields. As used herein, “plant tissue” also refers to a clone of a plant, seed, progeny, or propagule, whether generated sexually or asexually, and descendents of any of these, such as cuttings or seeds.

Unless indicated otherwise, as used herein, “altered expression of a coding region” refers to a change in the transcription of a coding region, a change in translation of an mRNA encoded by a coding region, or a change in the activity of a polypeptide encoded by the coding region. The change may be an increase or a decrease.

As used herein, “transformation” refers to a process by which a polynucleotide is inserted into the genome of a plant cell. Such an insertion includes stable introduction into the plant cell and transmission to progeny. Transformation also refers to transient insertion of a polynucleotide, wherein the resulting transformant transiently expresses a polypeptide that may be encoded by the polynucleotide.

As used herein, “phenotype” refers to a distinguishing feature or characteristic of a plant which can be altered according to the present invention by modifying expression of at least one coding region in at least one cell of a plant. The modified expression of at least one coding region can confer a change in the phenotype of a transformed plant by modifying any one or more of a number of genetic, molecular, biochemical, physiological, morphological, or agronomic characteristics or properties of the transformed plant cell or plant as a whole. Whether a phenotype of a transgenic plant is altered is determined by comparing the transformed plant with a plant of the same species that has not been transformed with the same polynucleotide (a “control plant”).

As used herein, “mutation” refers to a modification of the natural nucleotide sequence of a coding region or an operably linked regulatory region made by deleting, substituting, or adding a nucleotide(s) in such a way that the polypeptide encoded by the modified nucleic acid is altered structurally and/or functionally, or the coding region is expressed at a decreased or increased level.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably.

As used herein, a polypeptide may be “structurally similar” to a reference polypeptide if the amino acid sequence of the polypeptide possesses a specified amount of sequence similarity and/or sequence identity compared to the reference polypeptide. Thus, a polypeptide may be “structurally similar” to a reference polypeptide if, compared to the reference polypeptide, it possesses a sufficient level of amino acid sequence identity, amino acid sequence similarity, or a combination thereof.

As used herein, the tetra “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. A polynucleotide described herein may be isolated. An “isolated” polynucleotide is one that has been removed from its natural environment. Polynucleotides that are produced by recombinant, enzymatic, or chemical techniques are considered to be isolated and purified by definition, since they were never present in a natural environment.

A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

The term “complementary” refers to the ability of two single stranded polynucleotides to base pair with each other, where an adenine on one polynucleotide will base pair to a thymine or uracil on a second polynucleotide and a cytosine on one polynucleotide will base pair to a guanine on a second polynucleotide.

As used herein, “recalcitrance” refers to the natural resistance of plant cell walls to deconstruction by, for instance, microbial and/or enzymatic and/or chemical action (see Fu et al., 2011, Proc. Natl. Acad. Sci. USA 108:3803-3808).

Conditions that are “suitable” for an event to occur, or “suitable” conditions are conditions that do not prevent such events from occurring. Thus, these conditions permit, enhance, facilitate, and/or are conducive to the event.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description or in the claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Amino acid sequences of examples of hydroxyproline-rich glycopolypeptides and nucleotide sequences encoding same. Amino acid sequences (SEQ ID NO:2, 4, 6, 8, 10, 12, 14, and 16) and nucleotide sequences encoding the amino acid sequences (SEQ ID NO:1, 3, 5, 7, 9, 11, 13, and 15, respectively).

FIG. 2. Reverse phase HPLC chromatography of APAP1 (A and B) and separation of Hyp-O-glycosides from APAP1 by size exclusion chromatography (C and D). (A) Reverse phase PRP-1 chromatography profile of AGP-enriched material from Arabidopsis suspension culture medium (modified from Xu et al., 2008, Phytochemistry 69:1631-1640). AGPs from the medium of 10-day old Arabidopsis suspension cultured cells were purified by sequential DEAE anion exchange and Superose-12 size exclusion chromatography and separated into seven 220 nm-absorbing peaks (A) by reverse phase PRP-1 chromatography (Xu et al., 2008, Phytochemistry 69:1631-1640). The yield of peak 3 (5 mg/5 L media) was less than 1% w/w of the total AGPs recovered. (B) The material in Peak 3 from (A) was treated with Yariv reagent to yield a Yariv-precipitant and a Yariv-soluble fraction. The Yariv-soluble fraction (B) was separated by PRP-1 reverse phase chromatography into two subfractions, major peak YS1 (1 mg/5 L media) and shoulder peak YS2 (0.6-0.7 mg/5 L media). (C) YS 1 was hydrolyzed with 0.44 N NaOH at 105° C. for 18 hrs, each hydrolysate neutralized with 1N HCl on ice, and freeze-dried residues dissolved and separated over an analytical Superdex-75 column as described herein. An aliquot (10% volume) of each collected fraction was assayed for hydroxyproline (Hyp) and pentose. Squares represent absorbance at 560 nm to detect Hyp derivatives; diamonds represent absorbance at 665 nm to detect pentose derivatives. The asterisks show the fractions used for more extensive sugar analyses (fractions 22 and 50) and NMR analyses (fractions 26 and 27). (D) YS2 hydrolyzed and analyzed as described in (C).

FIG. 3. Protein sequence deduced from YS 1 and YS2 match exactly a portion of Arabidopsis gene, At3g45230/BAC43203. (A) The gene encodes a serine/proline-rich arabinogalactan protein (AGP), AtAGP57C (SEQ ID NO:2). N-terminal sequencing of YS1 yielded a 20 amino acid sequence that matched the underlined sequence of the mature serine/proline-rich protein. 0 stands for hydroxyproline as confirmed by amino acid sequencing. Double underlined sequence matched the N-terminal sequence of anhydrous HF deglycosylated YS2. (B) Domain structure diagram of AtAGP57C. TM1 and TM2 refer to two predicted transmembrane domains of APAP1 calculated by the transmembrane prediction programs TMHMM (available through the world wide web at cbs.dtu.dk/services/TMHMM/) and TMpred (available through the world wide web at ch.embnet.org/software/TMPRED_form.html). The TM1 regions include the predicted N-terminal TM from amino acids 5 to 22 and the predicted C-terminal TM, TM2, from amino acids 135 to 154. The regions at the ends of the protein and adjacent to the transmembrane domains are non-Pro/Hyp-rich regions, both predicted to have the opposite topology of the Pro/Hyp-rich domain. The N-terminal TM was predicted to be a signal peptide by SignalP (available through the world wide web at cbs.dtu.dk/services/SignalP). The C-terminal TM indicated a possible signal sequence for GPI-anchor modification (available through the world wide web at mendel.imp.ac.at/gpi/plant_server.html).

FIG. 4. ELISA analysis of YS2 using 47 monoclonal antibodies reactive against plant cell wall glycans. Forty seven plant cell wall glycan-reactive monoclonal antibodies (see Table 12 for information on antibodies) were used for enzyme-linked immunosorbent assay (ELISA) analysis of YS2 (Pattathil et al., 2010, Plant Physiol. 153:514-525, and Table 12). A total of 43 of the antibodies had absorbance signals greater than 0.1 and were identified as reactive against YS2/APAP 1. These antibodies were shown in previous studies to be reactive against epitopes in cell wall preparations enriched for the following cell wall polysaccharides and to group into the designated antibody-reactivity categories (Pattathil et al., 2010, Plant Physiol. 153:514-525): RG-I/AG, RG-I backbone, linseed mucilage RG-I, xylan-7, AG-2, and AG-4.

FIG. 5. NMR spectra of YS1-HP2627 collected at 25° C. on a Varian VNMRS 800 instrument. (A) HSQC spectrum. The insert is the enlarged anomeric region. Signals A and B were identified as the anomeric C/H of α-L-Araf residues; C, as the anomeric C/H of 1,3-galactan backbone Gal (Gal_{bb}); D, as the anomeric C/H of AG side chain Gal (Gals); E, as the anomeric C/H of β-D-Xylp residues; F, as the anomeric C/H of β-D-GlcAp; G, as the C/H-6 of α-L-Rhap residues (the signal of G is folded in this HSQC spectrum due to the small ¹³C spectral width used. As such, 80 ppm should be subtracted from the respective ¹³C chemical shifts); H, as the C/H-4 of 4-Xylp. (B) COSY spectrum (partial) collected at 25° C. The correlation between Hyp H-3ax and Hyp H-3ex is labeled. (C) TOCSY spectrum identified the protons of Xyl residues, GlcA, and the Gal residue attached to Hyp. The labels of X1 to X5 represent H-1 to H-5 of 4-Xyl; GA3 and GA5 as H-3 and H-5 of GlcA; G12 as H-2 of Gal attached to Hyp. A similar pattern was also observed in TOCSY recorded on YS2-HP2627. (D) NOESY spectrum (partial) (mixing time 100 ms). Cross-peak A shows the NOE between Xyl H-1 to Ara H-5, which established the β-D-Xylp-1→5-α-L-Araf linkage; cross-peak B shows NOEs between Gal_{bb} H1 and Gal_{bb} H3, suggesting the Gal_{bb}-1→3-Gal_{bb} linkage; crosspeak C(NOE between Gal_{bb} H1 and Gal_{bb} H6) supports the Gal_{bb}-1→6-Gal_{bb} linkage; NOEs in D suggest the Gals-1→6-Gal_{bb} linkage. (E) HMQC spectrum (partial) collected at 30° C. Signal A represents the anomeric ¹H/¹³C signals of 2-α-L-Rha, B the anomeric ¹H/¹³C signals of α-D-GalA, C to F the anomeric ¹H/¹³C signals of α-L-Araf. Hyp-glycoside YS1-HP2627 produced by SEC chromatography of base hydrolysed YS1 as depicted in FIG. 2C.

FIG. 6. NMR spectra of YS2-HP2627 collected at 25° C. on a Varian VNMRS 800 instrument. (A) HSQC spectrum. The insert is the enlarged anomeric region. Signal A represents anomeric H/C of α-D-GalAp; B, is H/C-1 of α-L-Rhap; C and D are H/C-1 of -α-L-Araf, E, F, G, H are H/C-1 of β-D-Xylp; I and J are H/C-1 of 1,3-galactan backbone Gal (Gal_{bb}); K is H/C-1 of 1,4-β-D-Galp; L is H/C-1 of AG side chain Gal (Gals); M is H/C-1 of GlcA; N is acetyl methyl group (the signals of N, Q, R, P are folded in this HSQC spectrum due to the small ¹³C spectral width used. As such, 80 ppm should be subtracted from the respective ¹³C chemical shifts); P, Q, and R are attributed to the methyl group of Rha residues. (B) NOESY spectrum (mixing time 100 ms). Signal A shows the NOE between GalA H-1 and Xyl H-1; C and D are the NOEs between Rha H-1 and Xyl H-1. These signals (A, C, and D) support the linkage between Xyl and Rha. Signal B represents the NOE between GalA H-1 and β-1,4-Galp H-1, indicating the attachment of β-1,4-galactan residues to Rha residues on RG-I.

FIG. 7. CID-MS/MS analysis of oligosaccharides released from YS2 by treatment with RGH indicates the presence of RG-I and HG domains in YS2 (A and B) and the existence of a covalent xylan to RG-I linkage in YS2 (C, D, and E). The MS2 fragmental Y and Z, B and C ions are consistent with the structures. (A) Rha-GalA-Rha-GalA-(GalA)₄-GalA-OAc for Rha₂GalA₇OAc (M+H+ Na+ m/z 1608); (B) Rha-GalA-Rha-GalA-(GalA)₃-GalA for Rha₂GalA₆ (M+H+Na+ m/z 1389); (C) Xylp-(Xylp)₃-Xylp-(AcO)Rha-GalA-Rha-GalA for Rha₂GalA₂Xyl₅OAc (M−H₂O+2H+m/z 1348); (D) Xylp-(Xylp)₂-Xylp-(AcO)Rha-GalA-Rha-GalA for Rha₂GalA₂Xyl₄OAc (M−H20+2H+ m/z 1216). These assignments are also based on the sugar linkage analyses and the cleavage features of RGH. These structures provide evidence that HG oligomers are flanked by RG-I oligomers in the pectin domain of APAP1 (A and B) and that some of the xylan oligomers are attached to Rha residues on RG-I sub-domains in APAP1 (C and D). (E) A representative MS2 spectrum of the RGH released oligosaccharides with the parent ion at m/z 1216. The Y, Z, B, and C ions, as well as some of the X ions that were labeled are indicated on the spectrum.

FIG. 8. NMR spectra of YS2 collected at 55° C. on a Bruker DRX800 instrument. (A) Anomeric region of HSQC spectrum. Signal A represents anomeric H/C of α-D-GalAp, B is H/C-1 of α-L-Rhap, C is H/C-1 of α-L-Araf, D is H/C-1 of 3-OAc-[2,4]-β-D-Xylp, E is H/C-1 of 3-OAc-[4]-β-DXylp, F is H/C-1 of 2,4-β-D-Xylp, G is H/C-1 of 4-β-D-Xylp, H and I are H/C-1 of 1→3-galactan Gal, J is H/C-1 of α-D-Galp on AG side chain, and K is H/C-1 of 4-β-D-Galp. (B) HMBC spectrum. Signal A to D show the HMBC connection between Xyl H-1 and other Xyl C-4. Signal E shows a correlation between carbonyl C of the OAc group and position 3 of Xylp, indicating the acetylation of Xyl. Signal E is a folded peak due to the small ¹³C spectral width used in data acquisition. Signal F confirmed the β-D-Gal-1→3-β-D-Gal linkage. Signal G established the β-D-Xylp-1→5-α-LAraf linkages. The t-Araf-1→2-β-D-Xylp connections in HMBC spectrum, between Araf H-1 at 5.1-5.3 ppm and Xylp C-2 at 82-84 ppm, were overlapped with correlations of Araf H-1 to Araf C-3 and Araf C-4.

FIG. 9. Proposed structural model for APAP1. RG-I is attached to type II AG through an α-D-GalA-1→2-α-L-Rha-1→4-β-D-GlcA-1→6-Gal structural unit (box 4). HG, with at least four to five GalA residues is embedded within RG-I (box 6). The length of flanking RG-I and HG domains is unknown. Arabinoxylan 1 is attached to type II AG through a β-D-Xylp-1→5-α-L-Araf-linkage (box 9). However, only one out of three possible types of Ara residues (based on the common sizes of arabinosyl side chains: Ara₁; Ara₂; and Ara₃) for this attachment is shown in this model. Furthermore, some of the Xyl residues in arabinoxylan 1 are arabinosylated at position 2. Arabinoxylan 2 is attached to RG-I through a β-D-Xylp-1→4-α-L-Rha linkage (box 10). The AGP protein core in APAP1 is AGP57C encoded by At3g45230. Numbered boxes (1 to 10) represent the identified structural units as listed in Table 10. Based on the amount of Hyp and monosaccharides in YS1 and YS2, a given Hyp residue in AtAGP57C in YS1 is attached with, on average, approximately 24 Gal, 45 Ara, 28 Xyl, 7 Rha and 13 GalA/GlcA while in YS2 there are 11 Gal, 65 Ara, 65 Xyl, 9 Rha and 13 GalA/GlcA residues. Due to the heterogeneity of glycosylation, this proposed model reflects representative, and not exact, numbers and length of the AG-pectin-(arabino)xylan chains in APAP1.

FIG. 10. Purification of Arabidopsis cell wall RG-1-enriched fraction. An RG-1-enriched fraction (Ara101) was prepared from cell walls of Arabidopsis suspension cultured cells using standard procedures (Guillaumie et al., 2003, Carbohydr. Res. 338:1951-1960; York et al., 1985, Methods Enzymol. 118:3-40), loaded onto a reverse phase column, and eluted using the same gradient as that used to purify YS1 and YS2 (see Methods, Example 1). The 220 nm-absorbing material that eluted at 20 min contained the RG-I containing APAP1-like complex. This fraction (Ara101P) was collected and used for further study.

FIG. 11. Analysis of APAP1 transcript level in apap1 mutants. (A). Relative positions of T-DNA inserts for apap1-3 and apap1-4, and primers for transcript expression level analysis of APAP1/AtAGP57C, the gene encoding the protein core of APAP1, are labeled with arrows. (B). Gel electrophoresis of semi-quantitative RT-PCR products from total RNA from leaves of apap1 homozygous and wild-type plants. The partial APAP1/AtAGP57C transcript (430 bp between primer 1 and 2 as labeled in A, out of the full length transcript of 498 bp) was absent in apap1-4, but was reduced in apap1-3. This result demonstrated that apap1-4 was a knockout line and apap1-3 was a knockdown line. The primers for APAP1/AtAGP57C are: Primer 1,5′-GAT GCT AAG TCT CGT ACT CGT C (SEQ ID NO:17); Primer 2,5′-CTC TTG CCG TTT CTT GTA CAC (SEQ ID NO:18); ACTIN 2 gene used as a control (71). The primers for ACTIN 2 included the sense primer 5′-ATC CTC CGT CTT GAC CTT GC (SEQ ID NO:19) and the antisense primer 5′-GAC CTG CCT CAT CAT ACT CG (SEQ ID NO:20). (C). Relative expression level of APAP1/AtAGP57C in wild type, apap1-3, and apap1-4 cauline leaves determined by real-time PCR. This result is consistent with the result shown in (B). There is a significant (˜31%) reduction in APAP1/AtAGP57C transcript in apap1-3, and no transcript detected in apap1-4. Relative expression level is presented as mean±standard deviation (n=4, ***p<0.001, Student's t-test), and the expression level of APAP1/AtAGP57C in wild type is set as 1. The data were analyzed as previously described (Livak and Schmittgen, 2001). The qPCR primer positions for APAP1/AtAGP57C are shown in (A) with sequences: q-primer-F, 5′-TCG CCG GAT TTG TGT ACA AG (SEQ ID NO:21); q-primer-R, 5′-AAT CTC TCT GGC GGC GTA AC (SEQ ID NO:22) (generating a 74 bp amplicon). ACTIN2 was used as the reference gene. The qPCR primers for ACTIN2 included the sense primer 5′-GGT AAC ATT GTG CTC AGT GGT GG (SEQ ID NO:23) and the antisense primer 5′-AAC GAC CTT AAT CTT CAT GCT GC (SEQ ID NO:24).

FIG. 12. Glycome profiling of sequential cell wall extracts from 8-week-old Arabidopsis thaliana apap1-3 and apap1-4 mutant and wild type plants. (A) Glycome profiling of wild type. Panels show analyses of representative examples from multiple biological replicates (See FIG. 13 for full set of results). Sequential cell wall extracts were prepared from the aerial portion (above the rosette leaves) of 8-week-old Arabidopsis thaliana wild type (Columbia-0) (A), apap1-3 mutant (B), and (C) apap1-4 mutant plants. Labels at the bottom show reagents used for the different extraction steps. The amounts of material extracted in each extraction step are indicated in the bar graphs at the tops of the heat maps. Extracts were ELISA-screened using 155 plant cell wall glycan-directed monoclonal antibodies (Table 12; Pattathil et al., 2010, Plant Physiol. 153:514-525). Data are represented as heatmaps. The panel on the right of the heatmaps shows the antibodies used, coded as groups based on the principal cell wall glycans recognized by each antibody group (Table 12; Pattathil et al., 2010, Plant Physiol. 153:514-525). Major changes in binding of specific antibodies to different mutant versus wild type wall extracts are outlined and correspond to xylan groups 3-7 and HG backbone-1 and RG-1 backbone. Strength of ELISA signal is indicated by the scale with bright depicting strongest binding and black indicating no binding. (B) Glycome profiling of apap1-3 mutant as described in (A). (C) Glycome profiling of apap1-4 mutant as described in (A).

FIG. 13. Glycome profiling of sequential cell wall extracts from 8-week-old Arabidopsis thaliana apap1-3 and apap1-4 mutant and wild type plants. (A-B) Glycome profiling of two different wild type plants. Sequential cell wall extracts were prepared from the aerial portion (above rosette leaves) of three biological replicates of 8-weekold Arabidopsis thaliana apap1-3 and apap1-4 mutant (Plants 1-3) and two biological replicates of wild type (Columbia-0) plants (Plants 1-2). Labels at the bottom show reagents used for the different extraction steps. The amounts of material extracted in each extraction step are indicated in the bar graphs at the tops of the heat maps. Extracts were ELISA-screened using 155 plant cell wall glycan-directed monoclonal antibodies. Data are presented as heatmaps. The panel on the right of the heatmaps shows the antibodies used, coded as groups based on the principal cell wall glycans recognized by each antibody group (Table 12). Strength of the ELISA signal is indicated by a scale with bright depicting the strongest binding and black indicating no binding. (C-E) Glycome profiling of two different apap1-3 mutants processed as described in (A-B). (F-H) Glycome profiling of two different apap1-4 mutants processed as described in (A-B).

FIG. 14. Difference heatmap of the glycome profiling data from 8-week-old Arabidopsis thaliana apap1 and wild type stems. The ELISA signals for the binding of each antibody to extracts from alcohol insoluble residue (AIR) from the apap1 mutant were subtracted from the corresponding ELISA signals for binding of the corresponding antibody to extracts from AIR from wild type plants. The resulting data are presented as a heatmap in which purple represents no difference between mutant and WT, black represents an increased signal in the mutant and yellow represents a reduced signal in the mutant. The heatmaps represent three biological replicates of 8-week-old Arabidopsis thaliana apap1-3 mutant (Plants 1-3) compared with two biological replicates of wild type (Columbia-0) plants (Plants 1-2) in all possible combinations. Differences observed in at least 5 out of the 6 individual difference heat maps were considered as indicative of mutant-associated changes in cell walls. The principal changes noted were changes in extractability of some glycan epitopes in mutant plants vs. WT plants. Notable differences (marked by white dotted lines) include: more HG and RG-I backbone epitopes eluting in the earliest extracted fraction (i.e. oxalate fraction) of the apap1 mutant; a reduced amount of RG-Ia epitope in later extracts from the apap1 mutant; less RG-I/AG epitopes in the 4M KOH extract and more of these epitopes in the chlorite extract from the apap1 mutant; reduced AG4 epitope in many extracts of the mutant plants.

FIG. 15. Measurement of stem length of Arabidopsis apap1-3/apap-14 mutants vs. wild type plants. Primary stem length of wild type, apap1-3 (A) and apap1-4 (B) mutant plants was measured on days 9 or 10 to 20 after planting on soil. Data are average growth rate±standard error measured as difference in stem length (cm) for each plant between the days indicated. The average stem length of apap1-3 (A) is (non-significantly) longer than that of wild type, while the average stem length of apap1-4 (B) is significantly longer than that of wild type from day 15 to day 17.

FIG. 16. Reverse phase purification of A104P. The detailed conditions were listed in the methods part of Example 1. The fraction with UV-220 nm absorbance at 27 min was named as A104P.

FIG. 17. Glycome-profiling of different extracts of either flowers or siliques from XG-5 mutants and WT plants. The alcohol insoluble residues of these tissues were extracted with increasing harsh solvent, including oxalate, carbonate, 1 M KOH, and 4 M KOH. The extracts were probed with antibodies raised against different cell wall polymers from the Plant Cell Wall Monoclonal Antibody Tool Kit. The 4 M KOH extract of mutant flowers has significant stronger binding to the antibodies against different xyloglucan antigens.

FIG. 18. Comparison of stem height of three selected APAP1-OE T1 plants, in which plant #1 has no stems, plant #2 has one modestly high stem (16 cm), and plant #3 has 6 WT-like stems (28 cm). All plants are 1-month-old after planting on soil.

FIG. 19. Semi-quantitative PCR of total RNA isolated from rosette leaf samples of five individual APAP1-OE T1 and 2 WT plants. RT-PCR products 300 bp in size were checked on a 1.2% agarose gel. Lanes 1 to 5 correspond to leaf samples from APAP1-OE T1 plants. Lanes 6 and 7 correspond to two leaf samples from WT plants. The stem number and heights of each APAP1-OE T1 plant were as follows: Lane 1, 0 cm; Lane 2, 1 stem, 14.5 cm; Lane 3, 3 stems, tallest 15 cm; Lane 4, 5 stems, tallest 27 cm; Lane 5, 5 stems, tallest 25.5 cm. WT plants: Lane 6, 6 stems, tallest 27 cm; Lane 7, 6 stems, tallest 25 cm. All plants were 1-month-old after planting on soil when measured.

FIG. 20. Comparison of siliques from WT, APAP1/AtAGP57C overexpression T1, and AP₅₁EGFP overexpression T1 plants. The AP₅₁EGFP gene is a synthetic gene, encoding a peptide of 51 repeats of Ala-Pro in which each Pro was posttranslationally hydroxylated and subsequently glycosylated with an arabinogalactan polysaccharide. The AP₅₁EGFP overexpression T1 plants were used as a control in this study. The severe APAP1-OE plants had short and sterile siliques, while WT and the control overexpression line AP₅₁EGFP-OE plants had normal siliques. Plants were 7-week-old.

### DETAILED DESCRIPTION OF ILLUSTRATIVE EMBODIMENTS

### Polypeptides

The present invention includes, but is not limited to, a transgenic plant having an alteration in expression of a coding region encoding a hydroxyproline-rich glycopolypeptide (HRGP). Examples of hydroxyproline-rich glycopolypeptides include arabinogalactan-protein (AGP) polypeptides, extensin polypeptides, proline-rich polypeptides, leucine-rich repeat extensin-like polypeptides, extensin-like polypeptides, and formin-like polypeptides. AGPs are a family of hydroxyproline-rich glycopolypeptides, and there are many members of this family (Showalter, 2001, CMLS Cell. Mol. Life. Sci., 58:1399-1417, Ellis et al., 2010, Plant Physiol., 153:403-419, and Showalter et al., 2010, Plant Physiol. 153:485-513).

One example of an AGP polypeptide is depicted at SEQ ID NO:2 (available through the Genbank database at accession number NP_{—}190109). Other examples of AGP polypeptides are depicted at SEQ ID NO:4 (available through the Genbank database at accession number EEE87468), SEQ ID NO:6 (available through the Genbank database at accession number EEE76032), SEQ ID NO:8 (available through the Genbank database at accession number AEE86796), and SEQ ID NO:10 (available through the Genbank database at accession number AEE30943).

One example of an extensin polypeptide is depicted at SEQ ID NO:14 (available through the Genbank database at accession number AEE76184).

One example of a proline-rich polypeptide is depicted at SEQ ID NO:12 (available through the Genbank database at accession number AAK63893).

One example of a formin-like polypeptide is depicted at SEQ ID NO:16 (available through the Genbank database at accession number Q6H7U3).

Other examples of HRGP polypeptides include those that are structurally similar the amino acid sequence of SEQ ID NO:2, SEQ ID NO:4, SEQ ID NO:6, SEQ ID NO:8, SEQ ID NO:10, SEQ ID NO:12, SEQ ID NO:14, or SEQ ID NO:16. For instance, other examples of AGP polypeptides include those that are structurally similar the amino acid sequence of SEQ ID NO:2, SEQ ID NO:4, SEQ ID NO:6, SEQ ID NO:8, or SEQ ID NO:10. Other examples of extensin polypeptides include those that are structurally similar the amino acid sequence of SEQ ID NO:12. Other examples of proline-rich polypeptides include those that are structurally similar the amino acid sequence of SEQ ID NO:14. Other examples of formin-like polypeptides include those that are structurally similar the amino acid sequence of SEQ ID NO:16.

Structural similarity of two polypeptides can be determined by aligning the residues of the two polypeptides (for example, a candidate polypeptide and any appropriate reference polypeptide described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference polypeptide may be a polypeptide described herein. A candidate polypeptide is the polypeptide being compared to the reference polypeptide. A candidate polypeptide may be isolated, for example, from a plant, or can be produced using recombinant techniques, or chemically or enzymatically synthesized. A candidate polypeptide may be inferred from a nucleotide sequence present in the genome of a plant.

Unless modified as otherwise described herein, a pair-wise comparison analysis of amino acid sequences can be carried out using the Blastp program of the BLAST 2 search algorithm, as described by Tatiana et al., (1999, FEMS Microbiol Lett, 174:247-250), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on. Alternatively, polypeptides may be compared using the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2.

Thus, as used herein, a candidate polypeptide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence.

Alternatively, as used herein, a candidate polypeptide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

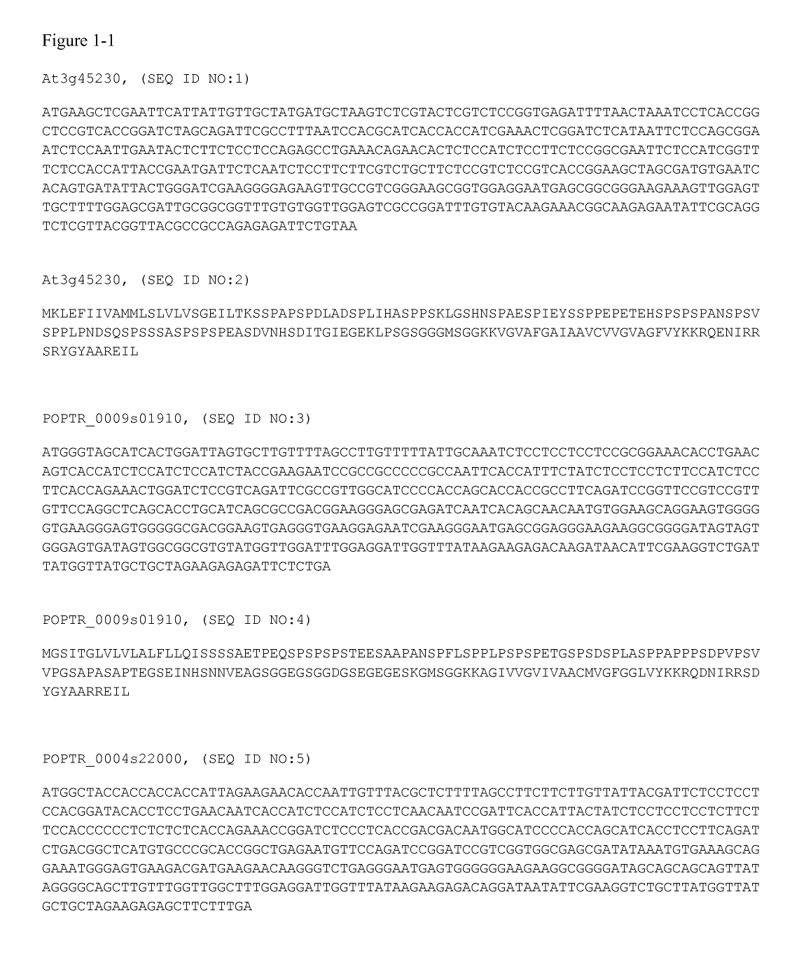

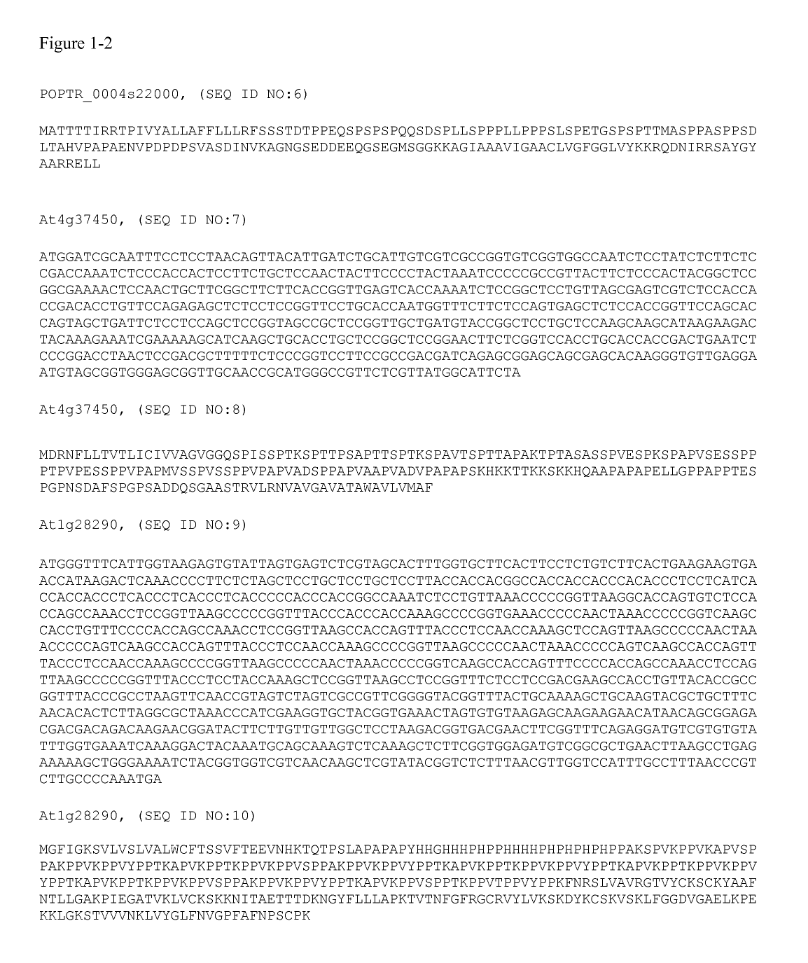

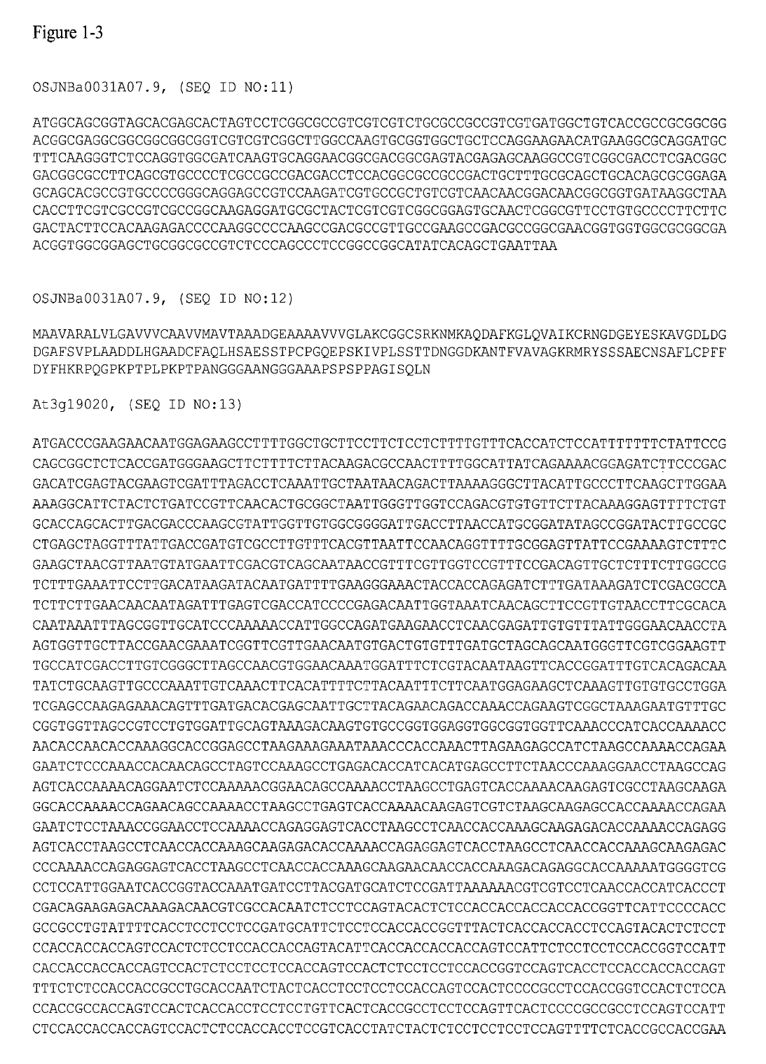

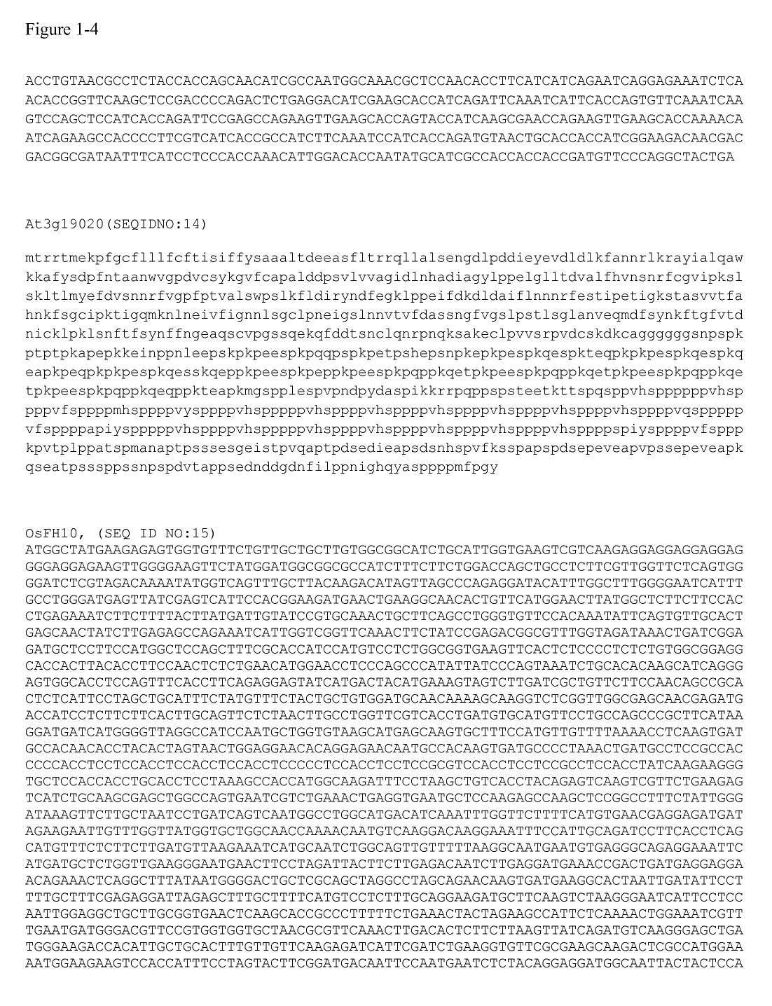

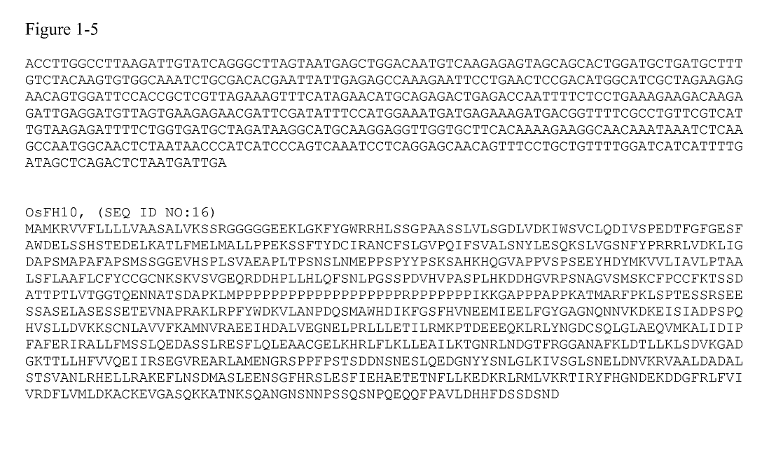

Among the HRGP superfamily, AGPs share the conserved repetitive AlaHyp, HypAla, SerHyp, and ThrHyp glycosylation motifs presented in their polypeptides. The polypeptides disclosed in FIG. 1 are shown having proline residues. The skilled person will understand that a proline present in a polypeptide may be post-translationally modified by hydroxylation to result in a hydroxyproline. Thus, reference to AlaHyp, HypAla, or other motifs having a hydroxyproline may appear in the primary sequence of a polypeptide as AlyPro, ProAla, etc. The Hyp residues in these motifs are usually glycosylated with large arabinogalactan polysaccharides. Other chimeric AGPs are composed both of these AGP glycosylation motifs and of other protein domains such as fasciclin-like or formin-like domains, which allow the bi- or multiple-features/functions of such AGPs (Showalter et al., 2010, Plant Physiology, 153: 485-513). Extensins have the characteristic SerHypHypHyp, SerHypHypHypHyp, and/or SerHypHypHypHypHyp repeats, in which the Hyp residues are the attachment sites of either single arabinose or oligoarabinosyl moieties (2 to 4 in a linear chain). Most extensins also share the TyrXTyr sequence, where X represents any amino acid usually except Pro. This sequence allows extensins to cross-link intramolecularly and intermolecularly to form covalently attached extensin network in plant cell wall. The proline residues in proline-rich proteins (PRPs) are usually the least hydroxylated, hence the least glycosylated (with single, 2 or 3 arabinosyl residues per chain) in the HRGP family PRPs contain variations of the pentapeptide ProHypValTyrLys, with both glycosylation site and cross-linking site. However, hybrid HRGPs are very common. Hybrid HRGP usually shares different characteristic amino acid sequences from different HRGPs in a single polypeptide. Other motifs are known to be present in HRGPs (see, for instance, Russinova and Reuseau, US Published Patent Application 20120331584).

### Polynucleotides

The present invention includes polynucleotides. Examples of polynucleotides encoding SEQ ID NO:2, SEQ ID NO:4, SEQ ID NO:6, SEQ ID NO:81, SEQ ID NO:10, SEQ ID NO:12, SEQ ID NO:14, and SEQ ID NO:16 are shown at SEQ ID NO:1, SEQ ID NO:3, SEQ ID NO:5, SEQ ID NO:7, SEQ ID NO:9, SEQ ID NO:11, SEQ ID NO:13, and SEQ ID NO:15 respectively. A nucleotide sequence of a polynucleotide encoding a polypeptide of the present invention may be readily determined by one skilled in the art by reference to the standard genetic code, wherein different nucleotide triplets (codons) are known to encode a specific amino acid. As is readily apparent to a skilled person, the class of nucleotide sequences that encode any polypeptide of the present invention is large as a result of the degeneracy of the genetic code, but it is also finite.

While the polynucleotide sequences described herein are listed as DNA sequences, it is understood that the complements, reverse sequences, and reverse complements of the DNA sequences can be easily determined by the skilled person. It is also understood that the sequences disclosed herein as DNA sequences can be converted from a DNA sequence to an RNA sequence by replacing each thymidine nucleotide with a uridine nucleotide.

Structural similarity of two polynucleotides can be determined by aligning the residues of the two polynucleotides (for example, a candidate polynucleotide and any appropriate reference polynucleotide described herein) to optimize the number of identical amino acids along the lengths of their sequences; gaps in either or both sequences are permitted in making the alignment in order to optimize the number of identical nucleotides, although the nucleotides in each sequence must nonetheless remain in their proper order. A reference polynucleotide may be a polynucleotide described herein. A candidate polynucleotide is the polynucleotide being compared to the reference polynucleotide. A candidate polynucleotide may be isolated, for example, from a plant, or can be produced using recombinant techniques, or chemically or enzymatically synthesized.

Unless modified as otherwise described herein, a pair-wise comparison analysis of nucleotide sequences can be carried out using the Blastn program of the BLAST search algorithm, available through the World Wide Web, for instance at the internet site maintained by the National Center for Biotechnology Information, National Institutes of Health. Preferably, the default values for all Blastn search parameters are used. Alternatively, sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

Thus, as used herein, a candidate polynucleotide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a reference amino acid sequence.

A polynucleotide of the present invention may further include additional nucleotides flanking the open reading frame encoding a polypeptide described herein. Typically, additional nucleotides may be at the 5′ end of the coding region, at the 3′ end of the coding region, or the combination thereof. The number of additional nucleotides may be, for instance, at least 10, at least 100, or at least 1000.

### Methods of Use

The present invention also provides methods of using hydroxyproline-rich glycopolypeptides and polynucleotides encoding hydroxyproline-rich glycopolypeptides. The hydroxyproline-rich glycopolypeptide may be, for instance, an AGP polypeptide, an extensin polypeptide, a proline-rich polypeptide, a leucine-rich repeat extensin-like polypeptide, an extensin-like polypeptide, or a formin-like polypeptide. The present invention includes methods for altering expression of plant hydroxyproline-rich glycopolypeptide coding regions for purposes including, but not limited to (i) investigating function of biosynthesis of pectin, xylan, and hydroxyproline-rich glycoproteins and ultimate effect on plant phenotype, (ii) effecting a change in plant phenotype, and (iii) using plants having an altered phenotype.

The present invention includes methods for altering the expression of a coding region encoding a hydroxyproline-rich glycopolypeptide. Thus, for example, the invention includes altering expression of an AGP coding region present in the genome of a wild-type plant. As disclosed herein, in one embodiment a wild-type plant is a woody plant, such as a member of the species Populus.

Techniques which can be used in accordance with the present invention to alter expression of a hydroxyproline-rich glycopolypeptide coding region include, but are not limited to: (i) disrupting a coding region's transcript, such as disrupting a coding region's mRNA transcript; (ii) disrupting the function of a polypeptide encoded by a coding region, (iii) disrupting the coding region itself, (iv) modifying the timing of expression of the coding region by placing it under the control of a non-native promoter, or (v) over-expression of the coding region. The use of antisense RNAs, ribozymes, double-stranded RNA interference (dsRNAi), and gene knockouts are valuable techniques for discovering the functional effects of a coding region and for generating plants with a phenotype that is different from a wild-type plant of the same species.

Antisense RNA, ribozyme, and dsRNAi technologies typically target RNA transcripts of coding regions, usually mRNA. Antisense RNA technology involves expressing in, or introducing into, a cell an RNA molecule (or RNA derivative) that is complementary to, or antisense to, sequences found in a particular mRNA in a cell. By associating with the mRNA, the antisense RNA can inhibit translation of the encoded gene product. The use of antisense technology to reduce or inhibit the expression of specific plant genes has been described, for example in European Patent Publication No. 271988, Smith et al., 1988, Nature, 334:724-726; Smith et. al., 1990, Plant Mol. Biol., 14:369-379.

A ribozyme is an RNA that has both a catalytic domain and a sequence that is complementary to a particular mRNA. The ribozyme functions by associating with the mRNA (through the complementary domain of the ribozyme) and then cleaving (degrading) the message using the catalytic domain

RNA interference (RNAi) involves a post-transcriptional gene silencing (PTGS) regulatory process, in which the steady-state level of a specific mRNA is reduced by sequence-specific degradation of the transcribed, usually fully processed mRNA without an alteration in the rate of de novo transcription of the target gene itself. The RNAi technique is discussed, for example, in Small, 2007, Curr. Opin. Biotechnol., 18:148-153; McGinnis, 1010, Brief Funct. Genomics, 9(2): 111-117.

Disruption of a coding region may be accomplished by T-DNA based inactivation. For instance, a T-DNA may be positioned within a polynucleotide coding region described herein, thereby disrupting expression of the encoded transcript and protein. T-DNA based inactivation can be used to introduce into a plant cell a mutation that alters expression of the coding region, e.g., decreases expression of a coding region or decreases activity of the polypeptide encoded by the coding region. For instance, mutations in a coding region and/or an operably linked regulatory region may be made by deleting, substituting, or adding a nucleotide(s). The use of T-DNA based inactiviation is discussed, for example, in Azpiroz-Leehan et al. (1997, Trends in Genetics, 13:152-156).

Over-expression of a coding region may be accomplished by cloning the coding region into an expression vector and introducing the vector into recipient cells. Alternatively, over-expression can be accomplished by introducing exogenous promoters into cells to drive expression of coding regions residing in the genome. The effect of over-expression of a given coding region on the phenotype of a plant can be evaluated by comparing plants over-expressing the coding region to control plants.

Altering expression of a coding region encoding a hydroxyproline-rich glycopolypeptide may be accomplished by using a portion of a polynucleotide described herein. In one embodiment, a polynucleotide for altering expression of a coding region in a plant cell includes one strand, referred to herein as the sense strand, of at least 19 nucleotides, for instance, 19, 20, 21, 22, 23, 24, 25, 26, 27, 28, or 29 nucleotides (e.g., lengths useful for dsRNAi and/or antisense RNA). In one embodiment, a polynucleotide for altering expression of a coding region in a plant cell includes substantially all of a coding region, or in some cases, an entire coding region (e.g., lengths useful for T-DNA based inactivation). The sense strand is substantially identical, preferably, identical, to a target coding region or a target mRNA. As used herein, the term “identical” means the nucleotide sequence of the sense strand has the same nucleotide sequence as a portion of the target coding region or the target mRNA. As used herein, the term “substantially identical” means the sequence of the sense strand differs from the sequence of a target mRNA at least 1%, 2%, 3%, 4%, or 5% of the nucleotides, and the remaining nucleotides are identical to the sequence of the mRNA.

In one embodiment, a polynucleotide for altering expression of a coding region encoding a hydroxyproline-rich glycopolypeptide in a plant cell includes one strand, referred to herein as the antisense strand. The antisense strand may be at least 19 nucleotides, for instance, 19, 20, 21, 22, 23, 24, 25, 26, 27, 28, or 29 nucleotides. In one embodiment, a polynucleotide for altering expression of a coding region in a plant cell includes substantially all of a coding region, or in some cases, an entire coding region. An antisense strand is substantially complementary, preferably, complementary, to a target coding region or a target mRNA. As used herein, the term “substantially complementary” means that at least 1%, 2%, 3%, 4%, or 5% of the nucleotides of the antisense strand are not complementary to a nucleotide sequence of a target coding region or a target mRNA.

Methods are readily available to aid in the choice of a series of nucleotides from a polynucleotide described herein. For instance, algorithms are available that permit selection of nucleotides that will function as dsRNAi and antisense RNA for use in altering expression of a coding region. The selection of nucleotides that can be used to selectively target a coding region for T-DNA based inactivation may be aided by knowledge of the nucleotide sequence of the target coding region.

Polynucleotides described herein, including nucleotide sequences which are a portion of a coding region described herein, may be operably linked to a regulatory sequence. An example of a regulatory region is a promoter. A promoter is a nucleic acid, such as DNA, that binds RNA polymerase and/or other transcription regulatory elements. A promoter facilitates or controls the transcription of DNA or RNA to generate an RNA molecule from a nucleic acid molecule that is operably linked to the promoter. The RNA can encode an antisense RNA molecule or a molecule useful in RNAi. Promoters useful in the invention include constitutive promoters, inducible promoters, and/or tissue preferred promoters for expression of a polynucleotide in a particular tissue or intracellular environment, examples of which are known to one of ordinary skill in the art.

Examples of useful constitutive plant promoters include, but are not limited to, the cauliflower mosaic virus (CaMV) 35S promoter, (Odel et al., 1985, Nature, 313:810), the nopaline synthase promoter (An et al., 1988, Plant Physiol., 88:547), and the octopine synthase promoter (Fromm et al., 1989, Plant Cell 1: 977).

Examples of inducible promoters include, but are not limited to, auxin-inducible promoters (Baumann et al., 1999, Plant Cell, 11:323-334), cytokinin-inducible promoters (Guevara-Garcia, 1998, Plant Mol. Biol., 38:743-753), and gibberellin-responsive promoters (Shi et al., 1998, Plant Mol. Biol., 38:1053-1060). Additionally, promoters responsive to heat, light, wounding, pathogen resistance, and chemicals such as methyl jasmonate or salicylic acid, can be used, as can tissue or cell-type specific promoters such as xylem-specific promoters (Lu et al., 2003, Plant Growth Regulation 41:279-286).

Another example of a regulatory region is a transcription terminator. Suitable transcription terminators are known in the art and include, for instance, a stretch of 5 consecutive thymidine nucleotides.

Thus, in one embodiment a polynucleotide that is operably linked to a regulatory sequence may be in an “antisense” orientation, the transcription of which produces a polynucleotide which can form secondary structures that affect expression of a target coding region in a plant cell. In another embodiment, the polynucleotide that is operably linked to a regulatory sequence may yield one or both strands of a double-stranded RNA product that initiates RNA interference of a target coding region in a plant cell.

A polynucleotide may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. A vector may result in integration into a cell's genomic DNA. A vector may be capable of replication in a bacterial host, for instance E. coli. Preferably the vector is a plasmid. In some embodiments, a polynucleotide can be present in a vector as two separate complementary polynucleotides, each of which can be expressed to yield a sense and an antisense strand of a dsRNA, or as a single polynucleotide containing a sense strand, an intervening spacer region, and an antisense strand, which can be expressed to yield an RNA polynucleotide having a sense and an antisense strand of the dsRNA.

Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include plant cells. Suitable prokaryotic cells include eubacteria, such as gram-negative organisms, for example, E. coli.

A selection marker is useful in identifying and selecting transformed plant cells or plants. Examples of such markers include, but are not limited to, a neomycin phosphotransferase (nptII) gene (Potrykus et al., 1985, Mol. Gen. Genet., 199:183-188), which confers kanamycin resistance. Cells expressing the nptII gene can be selected using an appropriate antibiotic such as kanamycin or G418. Other commonly used selectable markers include a mutant EPSP synthase gene (Hinchee et al., 1988, Bio/Technology 6:915-922), which confers glyphosate resistance; and a mutant acetolactate synthase gene (ALS), which confers imidazolinone or sulphonylurea resistance (Conner and Santino, 1985, European Patent Application 154,204).

Polynucleotides described herein can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide of the present invention in a cell, and the polynucleotide may then be isolated from the cell.

### Host Cells, Plants, and Transgenic Plants

The invention also provides host cells having altered expression of a coding region described herein. As used herein, a host cell includes the cell into which a polynucleotide described herein was introduced (a recombinant host cell), and its progeny, which may or may not include the polynucleotide. Accordingly, a host cell can be an individual cell, a cell culture, or cells that are part of an organism. The host cell can also be a portion of an embryo, endosperm, sperm or egg cell, or a fertilized egg. In one embodiment, the host cell is a plant cell.

The present invention further provides transgenic plants having altered expression of a coding region. A transgenic plant may be homozygous or heterozygous for a modification that results in altered expression of a coding region.

In one embodiment, a host cell is not an Arabidopsis thaliana cell. In one embodiment, a transgenic plant is not Arabidopsis thaliana. In one embodiment, a host cell or a transgenic plant may have a decrease in the amount of a hydroxyproline-rich glycopolypeptide. A host cell or transgenic plant having less of a hydroxyproline-rich glycopolypeptide may have a decrease of at least 1%, at least 5%, at least 10%, at least 20%, at least 30%, at least 40%, at least 50%, at least 60%, at least 70%, at least 80%, or at least 90% compared to the amount of the hydroxyproline-rich glycopolypeptide in a control plant. In one embodiment, a host cell or a transgenic plant may have a decrease in expression of a hydroxyproline-rich glycopolypeptide. In one embodiment, a host cell or a transgenic plant may have expression of an inactive hydroxyproline-rich glycopolypeptide. In one embodiment, a host cell or a transgenic plant may have expression of a hydroxyproline-rich glycopolypeptide that is altered to have decreased activity. A hydroxyproline-rich glycopolypeptide that is altered to have decreased activity may be decreased by at least 1%, at least 5%, at least 10%, at least 20%, at least 30%, at least 40%, at least 50%, at least 60%, at least 70%, at least 80%, or at least 90% compared to the activity of a hydroxyproline-rich glycopolypeptide in a control plant. In one embodiment, a host cell or a transgenic plant may have an absence of detectable expression of a hydroxyproline-rich glycopolypeptide.

In one embodiment, host cell or a transgenic plant may have an increase in the amount of a hydroxyproline-rich glycopolypeptide. A host cell or transgenic plant having more of a hydroxyproline-rich glycopolypeptide may have an increase of at least 1%, at least 5%, at least 10%, at least 20%, at least 30%, at least 40%, at least 50%, at least 60%, at least 70%, at least 80%, or at least 90% compared to the amount of the hydroxyproline-rich glycopolypeptide in a control plant. In one embodiment, a host cell or a transgenic plant may have an increase in expression of a hydroxyproline-rich glycopolypeptide. In one embodiment, a host cell or a transgenic plant may have expression of a hydroxyproline-rich glycopolypeptide that is altered to have increased activity. A hydroxyproline-rich glycopolypeptide that is altered to have increased activity may be increased by at least 1%, at least 5%, at least 10%, at least 20%, at least 30%, at least 40%, at least 50%, at least 60%, at least 70%, at least 80%, or at least 90% compared to the activity of a hydroxyproline-rich glycopolypeptide in a control plant.

The present invention also includes natural variants of plants, where the natural variants have increased or decreased expression of hydroxyproline-rich glycopolypeptide polypeptides, such as AGP polypeptides. In one embodiment, AGP expression is decreased. The change in AGP expression is relative to the level of expression of the AGP polypeptide in a natural population of the same species of plant. Natural populations include natural variants, and at a low level, extreme variants (Studer et al., 2011, 108:6300-6305). The level of expression of AGP polypeptide in an extreme variant may vary from the average level of expression of the AGP polypeptide in a natural population by at least 5%, at least 10%, at least 15%, at least 20%, or at least 25%. The average level of expression of the AGP polypeptide in a natural population may be determined by using at least 50 randomly chosen plants of the same species as the putative extreme variant.

The plants may be angiosperms or gymnosperms. The polynucleotides described herein may be used to transform a variety of plants, both monocotyledonous (e.g., grasses, corn, grains, oat, wheat, barley), dicotyledonous (e.g., Arabidopsis, tobacco, legumes, alfalfa, oaks, eucalyptus, maple, poplar, aspen, cottonwood), and Gymnosperms (e.g., Scots pine, white spruce, and larch).

The plants also include switchgrass, turfgrass, millets (such as foxtail millet), wheat, maize, rice, sugar beet, potato, tomato, lettuce, carrot, strawberry, cassava, sweet potato, geranium, soybean, and various types of woody plants. Woody plants include trees such as palm oak, pine, maple, fir, apple, fig, plum acacia, poplar, aspen, cottonwood, and willow. Woody plants also include rose and grape vines.

In one embodiment, the plants are woody plants, which are trees or shrubs whose stems live for a number of years and increase in diameter each year by the addition of woody tissue.

The invention includes plants of significance in the commercial biomass industry such as members of the family Salicaceae, such as Populus spp. (e.g., Populus trichocarpa, Populus deltoides), members of the family Pinaceae, such as Pinus spp. (e.g., Pinus taeda [Loblolly Pine]), and Eucalyptus spp.

Also provided is the plant material (such as, for instance, stems, branches, roots, leaves, fruit, etc.) derived from plant described herein. In one embodiment, the plant material is present in a plant material-derived product such as lumber (including, for instance, dimensional lumber and engineered lumber). In one embodiment, a plant material-derived product is a pulp. As used herein, “pulp” refers to a mechanically, chemically and/or biologically processed wood or non-wood plant material that contains cell wall material. Cell wall material includes cell walls, cell-wall polymers and/or molecules (such as oligosaccharides) that are derived from cell wall polymers. Cell wall polymers include cellulose, hemicellulose, pectin and/or lignin. Processing to generate a pulp may increase the susceptibility of the cell wall polysaccharides to hydrolysis and fermentation. Examples of pulp include, for instance, woodchips and sawdust. Also provided is pulp derived from a plant and/or plant material described herein.

Transformation of a plant with a polynucleotide described herein may yield a phenotype including, but not limited to, any one or more of changes in root growth, height, stem width, biomass yield, lignin quality, lignin structure, amount of lignin, pectin structure, hemicellulose structure, glycoconjugate structure, wood composition, wood strength, cellulose polymerization, fiber dimensions, cell wall composition (such as cell wall polysaccharide content), rate of wood formation, rate of growth, altered infloresence, leaf shape, wood flexibility, and wood strength. In one embodiment a phenotype is increased height compared to a control plant. In one embodiment a phenotype is reduced recalcitrance compared to a control plant. In one embodiment, decreased expression of a polynucleotide described herein may result in a plant having increased height and/or decreased recalcitrance compared to a control plant. In one embodiment a phenotype is shorter stems, smaller leaves, smaller inflorescences, smaller pollen, shorter roots, and/or reduced total biomass. In one embodiment, increased expression of a polynucleotide described herein may result in a plant having shorter stems, smaller leaves, smaller inflorescences, smaller pollen, shorter roots, and/or reduced total biomass. Methods for measuring recalcitrance are routine and include, but are not limited to, measuring changes in the extractability of carbohydrates, where an increase in extractability suggests a more loosely held together wall, and thus, decreased recalcitrance.

Other phenotypes present in a transgenic plant described herein may include yielding biomass with reduced recalcitrance and from which sugars can be released more efficiently for use in biofuel and biomaterial production, yielding biomass which is more easily deconstructed and allows more efficient use of wall structural polymers and components, and yielding biomass that will be less costly to refine for recovery of sugars and biomaterials. Other phenotypes present in a plant described herein, for instance, a transgenic plant with over-expression of an HRGP, may include biomass that is more cross-linked which may yield biomass with greater strength and altered biomechanical properties, biomass that is more dense, and/or biomass that yields biomaterials and sugars having altered characteristics when compared to a plant that does not have over-expression of an HRGP.

Phenotype can be assessed by any suitable means. The plants may be evaluated based on their general morphology. Transgenic plants can be observed with the naked eye, can be weighed and their height measured. The plant can be examined by isolating individual layers of plant tissue, e.g. phloem and cambium, and also by examining meristematic cells, early expansion tissue, late expansion tissue, and at secondary wall formation, late cell maturation and primary wall formation stages. The plants also can be assessed using microscopic analysis or chemical analysis.

Microscopic analysis includes examining cell types, stage of development, and stain uptake by tissues and cells. Fiber morphology, such as fiber wall thickness may be observed using, for example, microscopic transmission ellipsometry (Ye and Sundstrom, 1977, Tappi J., 80:181). Wood strength and density in wet wood and standing trees can be determined by measuring the visible and near infrared spectral data in conjunction with multivariate analysis (Gabor, U.S. Pat. No. 6,525,319). Lumen size can be measured using scanning electron microscopy. Lignin structure and chemical properties, (such as cell wall properties) can be observed using nuclear magnetic resonance spectroscopy, chemical derivatization, mass spectrometry, diverse microscopies, colorimetric assays, glycome profiling.

The biochemical characteristic of lignin, cellulose, carbohydrates and other plant extracts can be evaluated by standard analytical methods including spectrophotometry, fluorescence spectroscopy, HPLC, mass spectroscopy, molecular beam mass spectroscopy, near infrared spectroscopy, nuclear magnetic resonance spectroscopy, and tissue staining methods.

One method that can be used to evaluate the phenotype of a transgenic plant is glycome profiling. Glycome profiling gives information about the presence of carbohydrate structures in plant cell walls, including changes in the extractability of carbohydrates from cell walls (Zhu et al., 2010, Mol. Plant, 3:818-833; Pattathil et al., 2010, Plant Physiol., 153:514-525), the latter providing information about larger scale changes in wall structure. In one embodiment the change is an increase of one or more carbohydrates in an extracted fraction compared to a control plant. Examples of solvents useful for evaluating the extractability of carbohydrates include, but are not limited to, oxalate, carbonate, KOH (e.g., 1M and 4M), and chlorite. Diverse plant glycan-directed monoclonal antibodies are available from, for instance, CarboSource Services (Athens, Ga.), and PlantProbes (Leeds, UK).

In one embodiment, a transgenic plant has changes in glycoprotein recovery from extracts of alcohol insoluble residues of plant. The change may be an increase or a decrease of glycoprotein present in an extracted fraction compared to a control plant. In one embodiment, a transgenic plant has changes in the extractability of cell wall carbohydrates, such as arabinose, xylose, fuctose, rhamnose, glucose, galactose, mannose, glucuronic acid, galacturonic acid, or a combination thereof. The change may be an increase or a decrease of one or more of these carbohydrates in an extracted fraction compared to a control plant. In one embodiment the change is an increase of one or more of these carbohydrates in an extracted fraction compared to a control plant. In one embodiment the change is a decrease of one or more of these carbohydrates in an extracted fraction compared to a control plant. Examples of solvents useful for evaluating the extractability of glycoproteins and/or carbohydrates include, but are not limited to, oxalate, carbonate, KOH (e.g., 1M and 4M), and chlorite.

### Methods for Making

Transgenic plants described herein may be produced using routine methods. Methods for transformation and regeneration are known to the skilled person. Transformation of a plant cell with a polynucleotide described herein to yield a recombinant host cell may be achieved by any known method for the insertion of nucleic acid sequences into a prokaryotic or eukaryotic host cell, including Agrobacterium-mediated transformation protocols, viral infection, whiskers, electroporation, microinjection, polyethylene glycol-treatment, heat shock, lipofection, particle bombardment, and chloroplast transformation.

Transformation techniques for dicotyledons are known in the art and include Agrobacterium-based techniques and techniques that do not require Agrobacterium. Non-Agrobacterium techniques involve the uptake of exogenous genetic material directly by protoplasts or cells. This may be accomplished by PEG or electroporation mediated-uptake, particle bombardment-mediated delivery, or microinjection. In each case the transformed cells may be regenerated to whole plants using standard techniques known in the art.

Techniques for the transformation of monocotyledon species include direct gene transfer into protoplasts using PEG or electroporation techniques, particle bombardment into callus tissue or organized structures, as well as Agrobacterium-mediated transformation.

The cells that have been transformed may be grown into plants in accordance with conventional techniques. See, for example, McCormick et al. (1986, Plant Cell Reports, 5:81-84). These plants may then be grown and evaluated for expression of desired phenotypic characteristics. These plants may be either pollinated with the same transformed strain or different strains, and the resulting hybrid having desired phenotypic characteristics identified. Two or more generations may be grown to ensure that the desired phenotypic characteristics are stably maintained and inherited and then seeds harvested to ensure stability of the desired phenotypic characteristics have been achieved.

### Methods of Use

Provided herein are methods for using the plants and/or plant material described herein.

In one embodiment, a method includes using a plant and/or plant material. In one embodiment, a plant and/or plant material may be used to produce a plant material-derived product. Examples of plant material-derived products include pulp. Plant material-derived products, such as pulp, may be used as a food additive, a liquid absorbent, as animal bedding, and in gardening. Plants and/or plant material described herein may also be used as a feedstock for livestock. Plants with reduced recalcitrance are expected to be more easily digested by an animal and more efficiently converted into animal mass. Accordingly, in one embodiment, a method include using a plant and/or plant material described herein as a source for a feedstock, and includes a feedstock that has plant material from a transgenic plant as one of its components.

In one embodiment, the methods include producing a metabolic product. A process for producing a metabolic product from a transgenic plant described herein may include processing a plant (also referred to as pretreatment of a plant), enzymatic hydrolysis, fermentation, and/or recovery of the metabolic product. Each of these steps may be practiced separately, thus included herein are methods for processing a transgenic plant to result in a pulp, methods for hydrolyzing a pulp that contain cells from a transgenic plant, and methods for producing a metabolic product from a pulp.

There are numerous methods or combinations of methods known in the art and routinely used to process plants. The result of processing a plant is a pulp. Plant material, which can be any part of a plant, may be processed by any means, including, for instance, mechanical, chemical, biological, or a combination thereof. Mechanical pretreatment breaks down the size of plant material. Biomass from agricultural residues is often mechanically broken up during harvesting. Other types of mechanical processing include milling or aqueous/steam processing. Chipping or grinding may be used to typically produce particles between 0.2 and 30 mm in size. Methods used for plant materials may include intense physical pretreatments such as steam explosion and other such treatments (Peterson et al., U.S. Patent Application 20090093028). Common chemical pretreatment methods used for plant materials include, but are not limited to, dilute acid, alkaline, organic solvent, ammonia, sulfur dioxide, carbon dioxide or other chemicals to make the biomass more available to enzymes. Biological pretreatments are sometimes used in combination with chemical treatments to solubilize lignin in order to make cell wall polysaccharides more accessible to hydrolysis and fermentation. In one embodiment, a method for using transgenic plants described herein includes processing plant material to result in a pulp. In one embodiment, transgenic plants described herein, such as those with reduced recalcitrance and/or decreased lignification, are expected to require less processing than a control plant. In some embodiments, the conditions described below for different types of processing are expected to result in greater amounts of carbohydrate oligomers and carbohydrate monomers when used with a plant described herein compared to a control plant. The conditions described below for different types of processing are for a control plant, and the use of a plant as described herein is expected to require less severe conditions.

Steam explosion is a common method for pretreatment of plant biomass and increases the amount of cellulose available for enzymatic hydrolysis (Foody, U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process typically causes degradation of cell wall complex carbohydrates and lignin transformation. Addition of H₂SO₄, SO₂, or CO₂ to the steam explosion reaction can improve subsequent cellulose hydrolysis (Morjanoff and Gray, 1987, Biotechnol. Bioeng. 29:733-741).

In ammonia fiber explosion (AFEX) pretreatment, biomass is treated with approximately 1-2 kg ammonia per kg dry biomass for approximately 30 minutes at pressures of 1.5 to 2 MPa. (Dale, U.S. Pat. No. 4,600,590; Dale, U.S. Pat. No. 5,037,663; Mes-Hartree, et al. 1988, Appl. Microbiol. Biotechnol., 29:462-468). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun and Cheng, 2002, Bioresource Technol., 83:1-11).

Concentrated or dilute acids may also be used for pretreatment of plant biomass. H₂SO₄ and HCl have been used at high concentrations, for instance, greater than 70%. In addition to pretreatment, concentrated acid may also be used for hydrolysis of cellulose (Hester et al., U.S. Pat. No. 5,972,118). Dilute acids can be used at either high (>160° C.) or low (<160° C.) temperatures, although high temperature is preferred for cellulose hydrolysis (Sun and Cheng, 2002, Bioresource Technol., 83:1-11). H₂SO₄ and HCl at concentrations of 0.3 to 2% (wt/wt) and treatment times ranging from minutes to 2 hours or longer can be used for dilute acid pretreatment.

Hot water can also be used as a pretreatment of plant biomass (Studer et al, 2011, Proc. Natl. Acad. Sci., U.S.A., 108:6300-6305). In one embodiment, hydrothermal treatment is at a temperature between 130° C. and 200° C., such as 140° C., 160, or 180° C., and for a time between 5 minutes and 120 minutes. In one embodiment, examples of times include at least 5 minutes, at least 10 minutes, at least 20 minutes, at least 30 minutes, at least 45 minutes, or at least 60 minutes. In one embodiment, examples of times include no greater than 120 minutes, no greater than 105 minutes, no greater than 90 minutes, or no greater than 75 minutes. The temperature and time used depends upon the source and condition of the biomass used, and an effective combination of time and temperature can be easily determined by the skilled person. In one embodiment, the biomass is exposed to a hydrothermal pretreatment having a severity level of logR0 between 2 and 5, where severity is defined as R0=t*exp ((T−100)/14.73) with t the time in minutes and T the temperature in degree Celsius (Lloyd and Wyman, 2005, Bioresource Technology, 96(18):1967-1977; Overend and Chornet, 1987, Phil. Trans. R. Soc. Lond. (A321), 523-536; and Wyman and Kumar, US Published Patent Application 20110201084). Examples of severity levels include at least 2, at least 2.5, at least 3, at least 3.5, at least 4, at least 4.5, and at least 5.

Other pretreatments include alkaline hydrolysis (Qian et al., 2006, Appl. Biochem. Biotechnol., 134:273; Galbe and Zacchi, 2002, Appl. Microbiol. Biotechnol., 59:618), oxidative delignification, organosolv process (Pan et al., 2005, Biotechnol. Bioeng., 90:473; Pan et al., 2006, Biotechnol. Bioeng., 94:851; Pan et al., 2006, J. Agric. Food Chem., 54:5806; Pan et al., 2007, Appl. Biochem. Biotechnol., 137-140:367), or biological pretreatment.

Methods for hydrolyzing a pulp may include enzymatic hydrolysis. Enzymatic hydrolysis of processed biomass includes the use of cellulases. Some of the pretreatment processes described above include hydrolysis of complex carbohydrates, such as hemicellulose and cellulose, to monomer sugars. Others, such as organosolv, prepare the substrates so that they will be susceptible to hydrolysis. This hydrolysis step can in fact be part of the fermentation process if some methods, such as simultaneous saccharification and fermentation (SSF), are used. Otherwise, the pretreatment may be followed by enzymatic hydrolysis with cellulases.

A cellulase may be any enzyme involved in the degradation of the complex carbohydrates in plant cell walls to fermentable sugars, such as glucose, xylose, mannose, galactose, and arabinose. The cellulolytic enzyme may be a multicomponent enzyme preparation, e.g., cellulase, a monocomponent enzyme preparation, e.g., endoglucanase, cellobiohydrolase, glucohydrolase, beta-glucosidase, or a combination of multicomponent and monocomponent enzymes. The cellulolytic enzymes may have activity, e.g., hydrolyze cellulose, either in the acid, neutral, or alkaline pH-range.

A cellulase may be of fungal or bacterial origin, which may be obtainable or isolated from microorganisms which are known to be capable of producing cellulolytic enzymes. Useful cellulases may be produced by fermentation of the above-noted microbial strains on a nutrient medium containing suitable carbon and nitrogen sources and inorganic salts, using procedures known in the art.

Examples of cellulases suitable for use in the present invention include, but are not limited to, CELLUCLAST (available from Novozymes A/S) and NOVOZYME (available from Novozymes A/S). Other commercially available preparations including cellulase which may be used include CELLUZYME, CEREFLO and ULTRAFLO (Novozymes A/S), LAMINEX and SPEZYME CP (Genencor Int.), and ROHAMENT 7069 W (Rohm GmbH).

The hydrolysis/fermentation of plant material may require addition of cellulases (e.g., cellulases available from Novozymes A/S). Typically, cellulase enzymes may be added in amounts effective from 5 to 35 filter paper units of activity per gram of substrate, or, for instance, 0.001% to 5.0% wt. of solids. The amount of cellulases appropriate for the hydrolysis may be decreased by using a transgenic plant described herein. The amount of cellulases (e.g., cellulases available from Novozymes A/S) required for hydrolysis of the pretreated plant biomass may be decreased by at least 5%, at least 10%, at least 15%, at least 20%, at least 25%, at least 30%, or at least 40% compared to the amount of cellulases required for hydrolysis of a control plant. This decreased need for cellulases can result in a significant decrease in costs associated with producing metabolic products from plant materials.

The steps following pretreatment, e.g., hydrolysis and fermentation, can be performed separately or simultaneously. Conventional methods used to process the plant material in accordance with the methods disclosed herein are well understood to those skilled in the art. Detailed discussion of methods and protocols for the production of ethanol from biomass are reviewed in Wyman (1999, Annu. Rev. Energy Environ., 24:189-226), Gong et al. (1999, Adv. Biochem. Eng. Biotech., 65: 207-241), Sun and Cheng (2002, Bioresource Technol., 83:1-11), and Olsson and Hahn-Hagerdal (1996, Enzyme and Microb. Technol., 18:312-331). The methods of the present invention may be implemented using any conventional biomass processing apparatus (also referred to herein as a bioreactor) configured to operate in accordance with the invention. Such an apparatus may include a batch-stirred reactor, a continuous flow stirred reactor with ultrafiltration, a continuous plug-flow column reactor (Gusakov, A. V., and Sinitsyn, A. P., 1985, Enz. Microb. Technol., 7: 346-352), an attrition reactor (Ryu, S. K., and Lee, J. M., 1983, Biotechnol. Bioeng., 25: 53-65), or a reactor with intensive stirring induced by an electromagnetic field (Gusakov, A. V., Sinitsyn, A. P., Davydkin, I. Y., Davydkin, V. Y., Protas, O. V., 1996, Appl. Biochem. Biotechnol., 56: 141-153) Smaller scale fermentations may be conducted using, for instance, a flask.

The conventional methods include, but are not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC). The fermentation can be carried out by batch fermentation or by fed-batch fermentation.

SHF uses separate process steps to first enzymatically hydrolyze plant material to glucose and then ferment glucose to ethanol. In SSF, the enzymatic hydrolysis of plant material and the fermentation of glucose to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF includes the co-fermentation of multiple sugars (Sheehan, J., and Himmel, M., 1999, Enzymes, energy and the environment: A strategic perspective on the U.S. Department of Energy's research and development activities for bioethanol, Biotechnol. Prog., 15: 817-827). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., 2002, Microbiol. Mol. Biol. Reviews, 66: 506-577).

A method described herein may include recovery of the metabolic product. Examples of metabolic products include, but are not limited to, alcohols, such as ethanol, butanol, a diol, and organic acids such as lactic acid, acetic acid, formic acid, citric acid, oxalic acid, and uric acid. The method depends upon the metabolic product that is to be recovered, and methods for recovering metabolic products resulting from microbial fermentation of plant material are known to the skilled person and used routinely. For instance, when the metabolic product is ethanol, the ethanol may be distilled using conventional methods. For example, after fermentation the metabolic product, e.g., ethanol, may be separated from the fermented slurry. The slurry may be distilled to extract the ethanol, or the ethanol may be extracted from the fermented slurry by micro or membrane filtration techniques. Alternatively the fermentation product may be recovered by stripping.

The present invention is illustrated by the following examples. It is to be understood that the particular examples, materials, amounts, and procedures are to be interpreted broadly in accordance with the scope and spirit of the invention as set forth herein.

### Example 1

Plant cell walls are comprised largely of the polysaccharides cellulose, hemicellulose, and pectin along with ˜10% protein and up to 40% lignin. These wall polymers interact covalently and noncovalently to form the functional cell wall. Characterized cross-links in the wall include covalent linkages between wall glycoprotein extensins, between rhamnogalacturonan II monomer domains, and between polysaccharides and lignin phenolic residues. Here we show that two isoforms of a purified Arabidopsis arabinogalactan-protein (AGP) encoded by gene At3g45230/BAC43203C are covalently attached to wall matrix hemicellulosic and pectic polysaccharides, with rhamnogalacturonan I (RG I)/homogalacturonan (HG) linked to the rhamnosyl residue in the arabinogalactan (AG) of the AGP and with arabinoxylan attached to either a rhamnosyl residue in the RG I domain or directly to an arabinosyl residue in the AG glycan domain. The existence of this wall structure, named Arabinoxylan-Pectin-Arabinogalactan Protein 1 (APAP1), is contrary to prevailing cell wall models that depict separate protein, pectin and hemicellulose polysaccharide networks. The modified sugar composition and increased extractability of pectin and xylan immunoreactive epitopes in apap1 mutant aerial biomass support a role for the APAP1 proteoglycan in plant wall architecture and function.

### Methods

Isolation of YS1 and YS2 from Culture Media

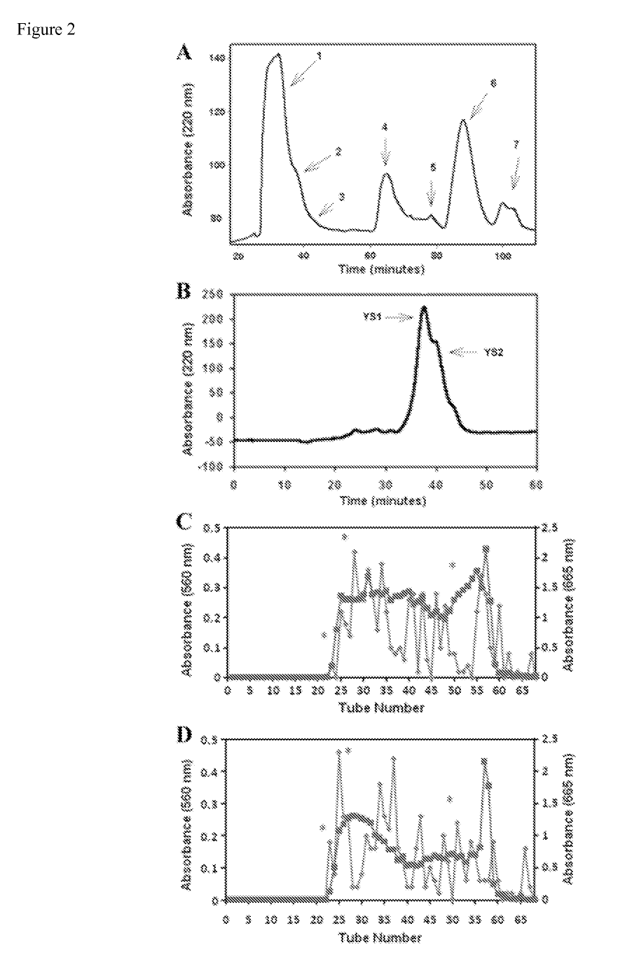

Arabidopsis thaliana (cv Columbia-0) cells were cultured as described earlier (Xu et al., 2008, Phytochemistry 69:1631-1640). AGP fractions from Arabidopsis culture media were purified and separated into seven subfractions by DEAE anion exchange (16·700 mm, Amersham-Pharmacia Biotech), Superose 12 size exclusion (16·500 mm, Amersham-Pharmacia Biotech), and PRP-1 reverse phase (10 μm, C-18, 7·305 mm, Hamilton) chromatographies as previously reported (Xu et al., 2008, Phytochemistry 69:1631-1640). The seven fractions were identified as containing AGPs based on precipitate formation upon addition of β-Gal Yariv-reagent to aliquots from each fraction. Fraction 3 from the PRP-1 column (see FIG. 2A) was shown to contain Xyl residues by glycosyl residue composition analysis of alditol acetates. The lyophilized fraction 3 (70 mg) was dissolved in 70 ml of water and mixed with an equal volume of 1 mg/ml β-galactosyl Yariv reagent. After incubation (25° C., 250 rpm) for 1 hr, the mixture was centrifuged (10,000×g, 10 min). The supernatant (termed Yariv soluble (YS)) was removed from the pellet (termed Yariv-precipitate (YP)) and dialyzed (10,000 MWCO, Spectrum) extensively against ddH₂O until the solution became colorless. The YS dialysate was lyophilized; dissolved in 3 ml Superose buffer (200 mM sodium phosphate, pH 7); and separated on a preparative Superose-12 column (16·500 mm, Amersham-Pharmacia Biotech, equilibrated with the same buffer at 1 ml/min). The column effluent was monitored by UV-detection and two fractions absorbing at 220 nm were detected and collected. The fraction representing the first peak was collected (YS-S12). The second peak was residual Yariv reagent.

The YS-S12 fraction was further purified on an analytical PRP-1 reverse phase column (5 μm, C-18, 4.1·150 mm, Hamilton), with a gradient from solvent A (0.1% (v/v) TFA aqueous) to 50% solvent B (80% (v/v) acetonitrile in 0.1% (v/v) TFA aqueous) over 100 min at 0.5 ml/min. Two fractions (Peak 1, designated YS1 and shoulder Peak 2, designated YS2 (Yariv soluble 1 and 2, respectively)) with UV absorbance at 220 nm were collected and freeze-dried. The material at the cross region between YS 1 and YS2 was not collected.

### Protein Deglycosylation Via Anhydrous HF Treatment

Freeze-dried YS2 (9 mg) was solubilized in 500 μl of anhydrous HF containing 10% (v/v) anhydrous methanol and the peptide deglycosylated on ice as previously described (Tan et al., 2003, Plant Physiol. 132:1362-1369).

### Automated Edman Degradation

N-terminal amino acid sequencing of the intact glycoprotein and the HF-deglycosylated protein was done on a 477A Applied Biosystems, Inc. gas phase sequencer at the Macromolecular Facility, Michigan State University (East Lancing, Mich.).

### Hyp-O-Glycoside Profiling

Fourteen mg of YS1 and 13.6 mg of YS2 were each hydrolyzed in 2 ml 0.44 N NaOH (105° C., 18 hr). The chilled hydrolysates were neutralized with 1M HCl, freeze-dried, redissolved in 500 μl of ddH₂O and loaded onto a ChromobeadsC2 cation exchange chromatography column (75×0.6 cm, Technicon) previously equilibrated with ddH₂O at 0.5 ml/min. Hyp-O-glycosides were separated and detected with an automated Hyp analyzer as previously described (Lamport and Miller, 1971, Plant Physiol. 48:454-456).

### Glycosyl Residue Composition and Linkage Analysis

For neutral sugar analysis, 60 μg of each sample was hydrolyzed in 200 μl of 2 N TFA at 121° C. for 1 hr. The hydrolysate was dried at 50° C. under a stream of N2 gas and the free sugars were converted to alditol acetate derivatives and analyzed by gas chromatography-FID as described (Tan et al., 2003, Plant Physiol. 132:1362-1369). Total uronic acids were measured colorimetrically using 100 μg of each sample with m-hydroxydiphenyl and using GlcUA as the standard as described earlier (Tan et al., 2003, Plant Physiol. 132:1362-1369).

A Dionex ICS-3000 HPLC system was used to determine the monosaccharide composition of some samples. The TFA hydrolysates were separated on a PA20 column (Dionex, Sunnyvale, Calif.) at high pH and detected using an electrochemical detector (ECD) in the carbohydrate mode. The buffers were Buffer A: nanopure water; Buffer B: 200 mM NaOH; and Buffer C, 1M NaOAc. The gradient was as follows: 0 min 1% buffer B; 0.1 min 10% buffer B; 2 min 10% buffer B; 4 min 1% buffer B; 15 min 0% buffer B; 25 min 5% buffer B and 10% buffer C; 30 min 5% buffer B and 50% buffer C; 35 min 1% buffer B. The flow rate was at 0.5 ml/min.

Total sugar analysis for quantification of both neutral and acidic sugars by GC-MS analysis of trimethylsilyl (TMS) derivatives and linkage analysis were conducted at the Complex Carbohydrate Research Center Analytical Services, University of Georgia. Glycosyl residue composition analysis was performed by combined GC/MS of per-O-TMS derivatives of monosaccharide methyl glycosides produced from the sample by acidic methanolysis. For glycosyl linkage analysis, the samples were permethylated, depolymerized, reduced, acetylated, and the resultant partially methylated alditol acetate residues analyzed by GC-MS (York et al., 1985, Methods Enzymol. 118:3-40).

### Fractionation of Hyp-O-Glycosides on Superdex-75 Column

Hyp-O-polysaccharides (750 μg) from YS1 or YS2 isolated following C2 Chromobeads cation exchange chromatography were dissolved in 1 ml of ddH₂O and further fractionated on an analytical Superdex-75 size exclusion column (10 mm i.d., 300 mm, Amersham-Pharmacia). The column eluent was acetonitrile/H₂O (20/80, v/v) at 0.3 ml/min and fractions (0.3 ml/fraction) were collected. All fractions were freeze-dried and those with abundant materials were analyzed for neutral sugar and uronic acid compositions (Tan et al., 2004, J. Biol. Chem. 279:13156-13165).

### β-Elimination of YS 1 and YS2

One mg each of YS 1 and YS2 was dissolved in 1 ml of 0.2 M NaOH in 1 M Na₂SO₃ solution. The mixtures were incubated at 50° C. for 5 hr, cooled on ice and neutralized with 0.1M HCl (Kieliszewski et al., 1992, Plant Physiol. 99:538-547). The glycoproteins after β-elimination were repurified on an analytical PRP-1 column as described above.

Preparation of RG-I from Cell Walls of Arabidopsis thaliana Suspension Cultured Cells

The RG-I preparation Ara101 was isolated from the walls of suspension-cultured Arabidopsis wild type cells (Guillaumie et al., 2003, Carbohydr. Res. 338:1951-1960) basically as described (York et al., 1985, Methods Enzymol. 118:3-40) with the following modifications. In brief, the cells were harvested and alcohol insoluble residue (AIR) of the cell walls was prepared. The dried AIR was deesterified overnight at pH 12 at 4° C. The reaction was stopped by lowering the pH to 5.2 with 1M HCl and the mixture dialyzed and lyophilized. The pectic polysaccharides were solubilized from the AIR by treatment with a purified Aspergillus niger endo-α-1,4-polygalacturonase (obtained from Carl Berfmann, CCRC) and the solubilized material was dialyzed and lyophilized. The resulting pectic mixture was fractionated in 50 mM sodium acetate (pH 5.2) by size-exclusion chromatography (SEC) on a Sephadex G-75 column (GE Healthcare Biosciences, Piscataway, N.J.) yielding three populations based on uronic acid colorimetric assays. The material in the largest-sized population size (named Ara101, RG-I) was pooled, dialyzed, and freeze dried.

Ten mg of Ara101 were dissolved in ddH₂O and purified on a reverse phase column using the same conditions as described above for YS1 and YS2. The resulting pectic proteoglycan was named Ara101P.

### Proteomic Analysis of Ara101P

One mg of Ara101P was deglycosylated with anhydrous HF as described above. The HF-treated material was dried under N2 and dissolved in 400 μl of 2% NH₄HCO₃ in 5 mM CaCl₂. A 30 μl aliquot of 0.1 μg/μl of sequence grade trypsin was added to the solution, followed by incubation at room temperature for 24 hrs. The tryptic peptides of HF-treated Ara101P were analyzed on an Agilent 1100 capillary LC (Palo Alto, Calif.) interfaced directly to an LTQ linear ion trap mass spectrometer (Thermo Electron, San Jose, Calif.). Buffers A and B were 0.1% formic acid in H₂O and 0.1% formic acid in acetonitrile, respectively. Peptides were eluted from a C18 column into the mass spectrometer during a 75 min linear gradient from 5 to 60% buffer B at a flow rate of 4 μl/min. The instrument was set to acquire MS/MS spectra on the nine most abundant precursor ions from each MS scan with a repeat count of 3 and repeat duration of 10 s. Dynamic exclusion was enabled for 200 s. Generated raw tandem mass spectra were converted into mzXML format and then into PKL format using ReAdW followed by mzMXL2Other (Pedrioli, et al., 2004, 22:1459-1466). The peak lists were searched using Mascot 2.2 software (Matrix Science, Boston, Mass.).

Database searches were performed against the annotated proteins from Arabidopsis genes obtained from NCBI (available through the world wide web at ncbi.nlm.nih.gov) using the following parameters: full tryptic enzymatic cleavage with two possible missed cleavages, peptide tolerance of 1000 parts-per million, fragment ion tolerance of 0.6 Da. Variable modification was set as carbamidomethyl due to carboxyamidomethylation of cysteine residues (+57 Da), oxidation of methionine residues (+16 Da) and hydroxylation of proline (+16 Da).

### Separation of Polygalacturonic Acid by Reverse Phase Chromatography

One mg of crude Sigma polygalacturonic acid (PGA) (P-1879, >66 kDa) was dissolved in 400 μl of ddH₂O and separated on an analytical PRP-1 reverse-phase column as described above. The polygalacturonic acid portion that voided on the column was collected and lyophilized.

### Attachment of YS2 to Magnetic Amine Beads

The covalent attachment of amino-containing material in YS2 onto BioMag Plus Amine beads (Bangs Laboratories, Inc.) was done following the manufacturer's manual. Briefly, the beads in their free amino group form were incubated with rotation with 5% (v/v) glutaraldehyde for 6 hr at room temperature on a rotary shaker. The supernatant was discarded and the beads were washed six times with pyridine wash buffer (10 mM pyridine adjusted to pH 6 with 6N HCl). YS2 (100 μg) was dissolved in 1 ml pyridine wash buffer, mixed with 1 mg of the above washed beads and incubated overnight at room temperature on a rotary shaker. Following incubation, the unbound material was removed and 0.5 mL of quenching solution (1M glycine) was added. The reaction was rotated for 1 hr at RT to block any un-reacted glutaraldehyde residues on the bead surface. The quenching solution was discarded and the beads were washed ten times with 1.5 ml wash buffer (10 mM Tris-HCl, pH 7.4, 0.1% (w/v) sodium azide, 0.1% (w/v) BSA, 1M NaCl, 10 mM M EDTA) and the sample stored in wash buffer at 4° C. until use.

### 2-Aminobenzamide Labeling

The reductive alkylation labeling of free reducing ends of YS2, YS2 Hyp-O-polysaccharide fraction 24, purified polygalacturonic acid (Sigma, P-1879), and an RG-1-enriched fraction (obtained from Dr. C. Deng, University of Georgia) was carried out by reaction with 2-aminobenzamide as described (Ishii et al., 2002, Carbohydr. Res. 337: 1023-1032). About 100 μg of each sample was used in the labeling reactions and after 1 hr at 80° C. the products from each reaction were purified on a Sephadex LH-20 column (1·30 cm, GE Healthcare, Piscataway, N.J.) to separate free 2-AB from labeled product. The labeled product was eluted by gravity using 20% (v/v) aqueous ethanol. The lyophilized 2-AB treated product was further purified on the analytical PRP-1 column as described above.

### Enzymatic Digestion of APAP 1

One hundred μg of YS2 was dissolved in 200 μl of 50 mM NaOAc, pH 5.5. One μl of the corresponding enzyme [rhamnogalacturonan hydrolase (Novozyme, 1 μg/μl) (Kofod et al., 1994, J. Biol. Chem. 269:29182-29189), endo-β-D-xylanase GH10 (CjXyn10A), endo-β-D-xylanase GH11 (NpXyn11A) and endo-β-D-xylanase GH5 (CtXy15A) (received from H. Gilbert)] was added individually to tubes and the tubes incubated at 37° C. for 24 hr on a rotary shaker. The released oligosaccharides voided on a PRP-1 analytical reverse phase column and were separated from the remaining proteoglycan using the conditions as described above.

### Collision-Induced Dissociation (CID)-MS/MS Analysis of YS2 Oligomers Released by Rhamnogalacturonan Hydrolase

The glycan fragments released from 100 μg of YS2 pre-treated with rhamnogalacturonan hydrolase (see above) were analyzed on a linear ion trap mass spectrometer (LTQ, ThermoFisher). The glycans were dissolved in a total of 50 μL of solvent (15 μL of 100% methanol and 35 μL of 1 mM NaOH in 50% methanol) and infused directly via a syringe pump (0.4 μl/min) into the mass spectrometer using a nanospray ion source with a fused-silica emitter (360×75×30 μm, SilicaTip™, New Objective) at 2.2 kV capillary voltage, 200° C. capillary temperature. Full ITMS (ion trap mass spectrometry) spectra carried out in positive mode and profile mode were collected between 400-2000 m/z for 30 sec with 5 microscans and 150 maximum injection time (ms). The centroid MS/MS spectra following collision-induced dissociation (CID) were obtained from 400 to 2000 m/z at 40% normalized collision energy, 0.25 activation Q, and 30.0 ms activation time by total ion mapping (TIM). Parent mass step size and isolation width were set at 2.0 m/z and 2.8 m/z, respectively, for automated MS/MS spectra with TIM scans.

### NMR Analysis

Five mg of YS2 were dissolved in 500 μl of 99.996% D₂O while 300 μg of YS1 Hyp-Opolysaccharides fraction 26 and 27 (YS1-HP2627) after separation on the Superdex 75 column as described above and 400 μg of YS2 Hyp-O-polysaccharide fraction 26 and 27 (YS2-HP2627) were dissolved in 150 μl of 99.996% D₂O. Samples were analyzed either on a Bruker-DRX800 equipped with a cryoprobe at the Campus Chemical and Instrument Center, Ohio State University, or on a Varian VNMRS 600 or VNMRS 800 equipped with a 3 mm cold probe at the Complex Carbohydrate Research Center, University of Georgia. NMR experiments were carried out either at 25° C. or higher temperature (30 to 55° C.). The 1D ¹H NMR, and 2D ₁H homonuclear COSY, TOCSY (DIPSI2 mixing time 60 millisecond), and NOESY (NOE mixing time 200 or 100 millisecond), 2D heteronuclear ¹H-¹³C HSQC, HMQC, and HMBC experiments were performed as described previously (Tan et al., 2010, J. Biol. Chem. 285:24575-24583).

Plant Genotyping, Semi-Quantitative PCR, and qPCR

Seeds from SALK T-DNA insertion lines SALK_{—}070113c/apap1-3 and SALK_{—}002144/apap1-4 were obtained from the Arabidopsis Biological Resource Center (available through the world wide web at biosci.ohiostate.edu/pcmb/Facilities/abrc/abrchome). Seeds from wild type Arabidopsis (C-0) and the two insertion lines were sterilized and germinated on ½ MS plates as described (Persson et al., 2007, Plant Cell 19:237-255). Two week old seedlings were transferred to soil and maintained in a 14/10 light/dark cycle (14 h at 19° C.; 150 μEi m⁻²s⁻¹/10 h at 15° C.).

Fresh leaf samples (1 cm²) from individual wild type and mutant plants were collected and DNA was isolated and genotyped using a REDExtract-N-Amp plant PCR kit (Sigma, St. Louis, Mo.). The following primers were used for PCR reactions: primers for SALK_{—}002144 (LP: CAAGTGTTTCGCACCTTTCTC (SEQ ID NO:25); RP: TTCATATCAAACAATATTTTTGAACTC (SEQ ID NO:26)), for SALK_{—}070113c (LP: GGTTCGGATATCTTTCGGTTC (SEQ ID NO:27); RP: GCAACTCCAACTTTCTTCCC (SEQ ID NO:28)), and the T-DNA primer LBb1.3 (ATTTTGCCGATTTCGGAAC (SEQ ID NO:29)). Total RNA was isolated from plant leaves using the RNeasy Plant Mini Kit (QIAGEN Sciences, Maryland). First-strand cDNA was synthesized from 850 ng of RNA using the SuperScript III First-Strand Synthesis Super Mix (Invitrogen, California) in reactions incubated for 50 min at 50° C. One microliter of the cDNA from each sample was amplified using primer 1, 5′-GAT GCT AAG TCT CGT ACT CGT C (SEQ ID NO:30) and primer 2,5′-CTC TTG CCG TTT CTT GTA CAC (SEQ ID NO:31), with an annealing temperature of 55° C., and 28 cycles of 95° C. 30 sec, 55° C. 30 sec, 72° C. 1 min.

The quantitative PCR was carried out with q-primer-F, 5′-TCG CCG GAT TTG TGT ACA AG (SEQ ID NO:32); q-primer-R, 5′-AAT CTC TCT GGC GGC GTA AC (SEQ ID NO:33) (generating a 74 bp amplicon) on a CFX96TM Real-Time PCR Detection System (Bio-Rad), following the manufacturer's instructions. ACTIN2 was used as the reference gene. The qPCR primers for ACTIN2 included the sense primer 5′-GGT AAC ATT GTG CTC AGT GGT GG (SEQ ID NO:34) and the antisense primer 5′-AAC GAC CTT AAT CTT CAT GCT GC (SEQ ID NO:35). Each reaction was repeated three times using cDNA generated from independently isolated RNA preparations. The PCR cycles included: initial polymerase activation at 95° C. for 3 min, then 40 cycles of 10 sec at 95° C. and 30 sec at 60° C. Melt curve analyses from 65° C. to 95° C. were performed after each run to ensure single size amplicon production. Data are mean±standard deviation of two biological samples. The data were analyzed as described by Livak and Schmittgen (2001, Methods 25:402-408.).

### Cell Wall Fractionation for Glycome Profiling

Fresh samples (all plant materials above rosette leaves) from each plant were collected and ground immediately under liquid nitrogen to a fine powder. Each powder was sequentially washed with 80% (v/v) EtOH, 95% (v/v) EtOH, CHCl₃/MeOH (1:1, v/v), and acetone with agitation. The insoluble residues (Alcohol Insoluble Residues, AIR) were air-dried in a hood. Sequential extraction of cell walls (AIR) was done with increasingly harsh reagents in the following order: 50 mM ammonium oxalate (Oxalate), 50 mM sodium carbonate with 0.5% (w/v) of sodium borohydride (Carbonate), 1M KOH with 1% (w/v) of sodium borohydride, 4M KOH with 1% (w/v) of sodium borohydride, 100 mM sodium chlorite (Chlorite) and post-chlorite 4M KOH with 1% (w/v) of sodium borohydride treatment (4M KOH PC) to isolate fractions enriched in cell wall components as previously described (Zhu et al., 2010, Mol. Plant. 3:818-833). The 1M KOH, 4M KOH and 4M KOH PC fractions were neutralized using glacial acetic acid. All extracts were dialyzed with four changes of de-ionized water (sample:water ˜1:60) at room temperature for a total of 48 hours and then lyophilized.

Total sugars in the cell wall fractions were quantified using the phenol sulphuric acid method (Masuko et al., 2005, Anal. Biochem. 339:69-72) and ELISA analyses were done as previously described (Pattathil et al., 2010, Plant Physiol. 153:514-525; Zhu et al., 2010).

### Monoclonal Antibodies

Monoclonal antibodies against different cell wall glycans were obtained as hybridoma cell culture supernatants from laboratory stocks at the Complex Carbohydrate Research Center. CCRC, JIM and MAC series of antibodies are available from CarboSource Services (carbosource.net). LAMP and BG-1 antibodies are available from Biosupplies Australia, Parkville, Victoria, Australia and were used as per the manufacturers' instructions. ELISA analyses of YS2 with different antibodies were done as previously described (Pattathil et al., 2010, Plant Physiol. 153:514-525; Zhu et al., 2010).

### Results

### Identification of a Xyl-Rich and GalA-Containing AGP

During an extensive purification and analysis of AGPs from Arabidopsis suspension culture medium, seven AGP-containing protein fractions were collected (FIG. 2A). Neutral sugar and uronic acid analyses showed that the major glycosyl residues in each fraction were those expected for AGPs (see Table 1). These analyses also revealed that fraction 3 (Peak 3 in FIG. 2A) contained 45% (molar percentage) xylose (see Table 1), an uncharacteristically high amount of xylose for AGPs. Attempts to purify the xylose rich material from AGPs in fraction 3 by precipitation with β-Gal Yariv reagent, a method that precipitates most AGPs, resulted in the recovery of the xylose-rich material in the Yariv soluble fraction and this fraction was named YS. The Yariv solubility of YS suggested either that YS did not contain an AGP or that one or more AGPs in YS had unusual glycosylation or glycan modifications and thus, was not accessible for precipitation with Yariv reagent. The YS fraction was further purified by size exclusion and reverse phase chromatography into two YS populations: a major UV-220 nm absorbing protein fraction (abbreviated YS1) and a minor fraction (YS2) (FIG. 2B). Both YS1 and YS2 eluted during Superose-12 gel chromatography with a molecular size between 75 to 100 kDa.

TABLE 1
Glycosyl residue composition (mole percentage) of neutral and acidic sugars
in Arabidopsis AGPs isolated by a combination of anion exchange, gel filtration
and reverse phase liquid chromatography. Final chromatography profile is
shown in FIG. 1A. Peak numbers denote the fraction numbers depicted in FIG.
1A. Material in At peak3 contained high levels of Xyl residues.

| Glycosyl | At^{a} | At | At | At^{a} | At | At^{a} | At^{a} |
|---|---|---|---|---|---|---|---|
| residue | Peak 1 | Peak 2 | Peak 3 | Peak 4 | Peak 5 | Peak 6 | Peak 7 |
| Rha | trace | 2 | 6 | 3 | 2 | 0 | 0 |
| Ara | 38 | 40 | 27 | 37 | 38 | 41 | 32 |
| Gal | 53 | 42 | 11 | 49 | 52 | 48 | 55 |
| Uronic acid | 9 | 11 | 11 | 11 | 8 | 11 | 10 |
| Fuc | 0 | 0 | 0 | 0 | 0 | 0 | 0 |
| Xyl | 0 | trace | 45 | 0 | 0 | 0 | 0 |
| Man | 0 | 0 | 0 | 0 | trace | trace | 3 |

^{a}Taken from Xu et al., 2008, Phytochemistry 69: 1631-1640.

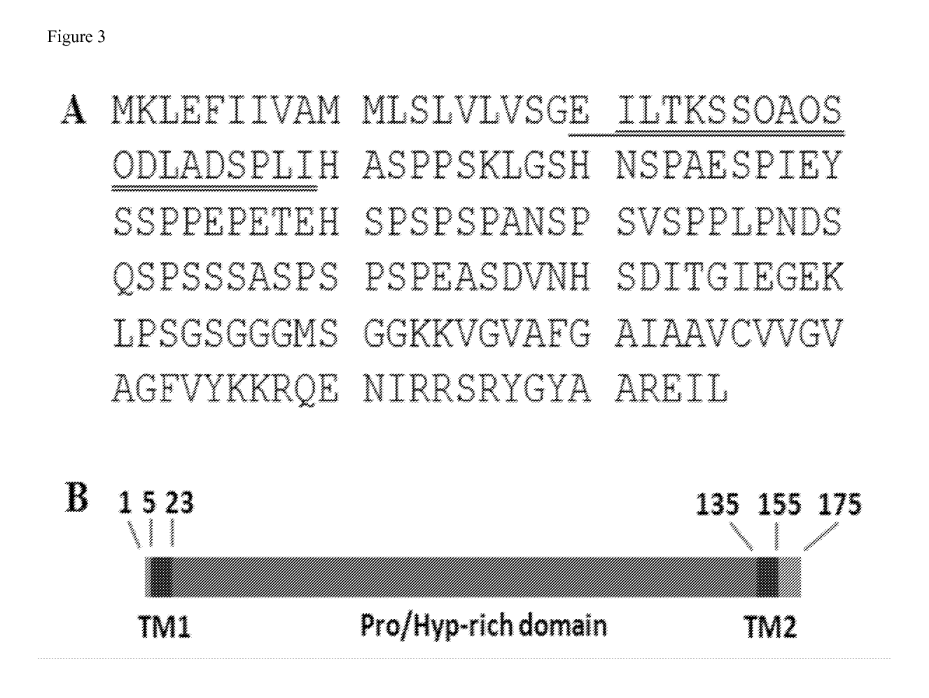

To identify any protein component in YS1 and YS2, native YS1 and anhydrous HF-deglycosylated YS2 were subjected to N-terminal amino acid sequencing, yielding an N-terminal amino acid sequence of EILTKSSOAOSODLADSPLI (0 as Hyp) (SEQ ID NO:36) for YS1 and an almost identical N-terminal sequence of (ILTKSSOAOSODLADSPLI (SEQ ID NO:37)) for YS2. A comparison of these sequences with the translated Arabidopsis genome database revealed a perfect match with Arabidopsis protein (At3g45230/BAC43203) which is annotated as hydroxyproline-rich glycoprotein family protein and as arabinogalactan protein AGP57C (Showalter et al., 2010, Plant Physiol. 153:485-513) (FIG. 3). The identified N-terminal sequences of YS 1 and YS2 represent cleavage at amino acid 19 (G) and 20 (E), respectively, of the predicted 175 amino acid protein encoded by gene At3g45230 (FIG. 3). In agreement with this observation, the protein encoded by At3g45230 is predicted by PSORT (available through the world wide web at psort.hgc.jp) to have a potential signal sequence cleavage site at amino acid 19. The PSORT and TmHMM_V2 (available through the world wide web at cbs.dtu.dk/services/TMHMM/) programs also predict an alpha helix membrane spanning domain between C-terminal amino acids 135 and 154, suggesting a possible signal sequence for GPI-anchoring. Taken together, these results show that YS 1 and YS2 contain the same AGP protein core.

Sugar composition analyses revealed that YS1 and YS2 contained, respectively, 63% and 97% sugar residues (w/w) including galacturonic acid (GalA) and Xyl (Table 8). This high sugar content explained why these relatively small polypeptides (FIG. 3) eluted as much larger proteins (75-100 kDa) upon size exclusion chromatography. The presence of high levels of Xyl and GalA in YS 1 and YS2 (Table 8) was intriguing because these sugars are commonly present in xylan and pectin, respectively, but are not generally found in these amounts in ‘typical’ AGPs. Sugar linkage analyses confirmed the presence of type II arabinogalactan (AG), based on the existence of terminal Galp and 3-, 6-, 3,6-Galp in YS1 and YS2 (Table 8), and also confirmed the identity of these proteoglycans as AGPs. However, the glycosyl linkage analyses also indicated the existence of 2- and 2,4-Rhap, 4-GalAp, and 4- and 2,4-Xylp, indicating the presence of the pectins RG-I, HG and of substituted xylan in the YS1 and YS2 preparations. This led to the question of whether the pectin and xylan-like material was covalently attached to the AGP in YS 1 and YS2, or alternatively, whether free pectin and xylan polysaccharides had cofractionated with the AGPs.

### Evidence for Covalent Attachment of Xylan and Pectin to the AGP in YS

Two methods were used to determine whether the xylan- and pectin-like glycans in YS were free polysaccharides or covalently attached to the AGPs. If free pectin and/or xylan polysaccharides were present in YS they would be detectable by reducing end assays or reducing end labeling due to the presence of terminal anomeric carbons not bound in a glycosidic bond. The reducing end of polysaccharides can be labeled with the hydrophobic fluor 2-amino-benzoamide (2-AB) and detected by a gain in absorbance at UV-254 nm (Ishii et al., 2002, Carbohydr. Res. 337:1023-1032). Following incubation of YS2 with 2-AB and purification of the treated-YS2 by size exclusion and reverse phase chromatography, the resulting 2-AB-treated YS2 showed no gain in absorbance at 254 nm and had the same retention time as non-treated YS2. These results indicated that there was no covalent attachment of the hydrophobic 2-AB to any component in YS2. Conversely, 2-AB labeling of the control polysaccharide, polygalacturonic acid (PGA), resulted in a gain of UV absorbance at 254 nm. In addition, 2-AB labeling caused the PGA to bind to the reverse phase column while non-labeled PGA did not, indicating increased hydrophobicity upon the attachment of the hydrophobic 2-AB to the control PGA. Finally, sugar composition analyses of 2-AB treated and non-treated YS2 after sequential size exclusion and reverse phase chromatography indicated that 2-AB labeling resulted in no change in sugar composition of 2-AB-treated YS2 compared to YS2. The results indicated that there were no free reducing ends present in YS2 and did not support the presence of any free pectin or xylan polysaccharides in the YS2 preparation.

Another method used to test if the pectin and xylan were covalently attached to the YS AGP involved selective attachment of the protein component of the AGP to a resin and the washing away any non-bound material. The AGP protein component in YS2 was covalently bound to glutaraldehyde-activated amine magnetic beads via a Schiff base reaction with the primary amine groups in the N-terminal and intrapeptide lysine residues present in the protein. The YS2-beads were extensively washed with wash buffer (10 mM EDTA, 1 M NaCl, 0.1% BSA) to dissociate non-covalently bound material and the washed YS2-beads hydrolyzed in 2N TFA to release monosaccharides that had been covalently attached to the protein. Glycosyl residue composition analysis revealed that the sugar component on the YS2-beads was identical to that of the starting YS2 material (See Table 11). The lack of any loss of sugar upon washing of the bound beads indicated that there were no free polysaccharides present in YS2. Control reactions in which a monosaccharide mixture (Ara, Gal, Xyl, GalA, Rha) and a yeast 1,3; 1,6-glucan were incubated with activated beads, as well as incubation of YS2 with nonactivated beads, resulted in no sugar residues being bound to the beads. These data confirmed that there were no free polysaccharides present in the YS2 preparation and that the glycans in YS2 were covalently attached to the protein. Taken together these results supported the conclusion that the xylan and pectin-like glycans, AG, and polypeptide moieties in YS2 were covalently linked together.

Xylan in YS is Arabinoxylan that is Attached in Two Different Ways to the AGP with Approximately Half of the Xylan Attached to the RG-I Domain

To probe the identity and structure of the glycans in relation to the typical AGP core, YS2 was hydrolyzed using glycan-specific enzymes. Rhamnogalacturonan hydrolase (RGH) is an endohydrolytic enzyme that cleaves the repeating [-2-α-L-Rha-1→4-α-D-GalA-1→] linkages present in the backbone of RG-I, yielding fragments with Rha at the non-reducing end. Because RG-I may contain 1,5-arabinan, 1,4-galactan, and/or type II AG side chains, the RGH treatment was also predicted to release arabinosyl and galactosyl-containing RG-I fragments from YS2. Indeed, treatment of YS2 with RGH released more than 90% of the Rha, 90% of the GalA, 61% of the Ara, and 35% of the Gal residues from YS2 (see Table 2). These results provided initial evidence that the xylose-rich YS2 contained a glycan structure typical of RG-I. There was also a concomitant release of 44% of the Xyl residues upon cleavage of YS2 with RGH. Because almost half of the xylan in YS2 was released by the RGH treatment, while about half remained associated with the remaining YS2, we postulated that some xylan-like structure was linked to RG-I and some xylan-like structure was linked to the remaining part of YS2 in a different manner (i.e. either to the AG portion of the AGP or to the protein backbone of the AGP).

TABLE 2
Sugar composition of YS2 before and after treatment with rhamnogalacturonan hydrolase
and endo-β-D-xylanase GH10. Rhamnogalacturonan hydrolase released most of the Rha
and GalA from YS2. Xylanase released 33.2% of Xyl from YS2. For each assay, 100 μg of
YS2 were used for digestion. Data are nmol released from 100 μg of sample.

| | RGH | | Combined data | | | Combined data of |
|---|---|---|---|---|---|---|
| | released | | of RGH-treated | Xylanase | | xylanase-treated |
| | oligomers | YS2 after | YS2 and the | released | YS2 after | YS2 and the |
| Glycosyl | from | RGH | released | oligomers | Xylanase | released |
| Residue | YS2 | treatment | oligomers | from YS2 | treatment | oligomers |
| Ara | 141.7 | 89.0 | 230.7 | 36.8 | 182.0 | 218.8 |
| Gal | 15.0 | 28.0 | 43.0 | — | 47.9 | 47.9 |
| Rha | 29.7 | — | 29.7 | — | 31.9 | 31.9 |
| Xyl | 110.6 | 143.1 | 253.7 | 83.0 | 166.8 | 249.8 |
| GalA | 55.7 | — | 55.7 | — | 53.0 | 53.0 |
| GlcA | 0.6 | 7.0 | 7.6 | 4.3 | 4.0 | 8.3 |

Glycosyl residue linkage analyses indicated that the GalA residues in YS2 were 4-linked and that the Rha residues were either 2-linked or 2,4-linked. Thus, one possible attachment site of xylan to RG-I was at the 4-position of Rha in the RG-I backbone. The attachment of short β-1,4-linked xylose oligosaccharides to the 4-position of the RG-I backbone has previously been reported in soybean (Nakamura et al., 2002, Biosci. Biotechnol. Biochem. 66:1155-1158), but there have been no prior reports of this structure associated with AGPs.

After RGH treatment and reverse phase column purification of RGH-treated YS2 (See Table 2), the treated YS2 was able to be precipitated with β-Gal Yariv reagent. These results indicated that the Yariv-precipitable AG glycan portion of the AGP was made accessible following removal of the pectin and xylan-like glycans upon RGH-treatment, thereby resulting in precipitation of the treated YS2 (xylose-containing AGP) by Yariv reagent. The fully glycosylated form of YS2 prior to RGH treatment was resistant to precipitation. These results provided additional support for the covalent attachment of xylan and RG-I glycans to the AGP.

To investigate the YS2 structure further, untreated YS2 was incubated with three endo-β-D-xylanases. Approximately one third of the xylose was released from YS2 upon treatment with either of two different endo-β-D-xylanases: CAZy (Cantarel at al., 2009, Nucleic Acids Res. 37:D233-238) family GH10 xylanase CjXyn10A (Biely et al., 1997, J. Biotechnol. 57:151-166.) and GH11 xylanase NpXyn11A (Paës et al., 2012, Biotechnol Adv. 30:564-92) (See Table 2). These enzymes hydrolyze low-substituted xylan and require at least two or three, respectively, unsubstituted xylose residues for cleavage. The results suggested that 33% of the xylan in YS2 was low to moderately substituted while the remaining ˜67% was more highly substituted, which was consistent with the 22% of terminal-Araf in the linkage analysis of YS2 (Table 8). However YS2 was not cleaved by a GH5 family endo-β-D-xylanase (CtXyl5A) (Correia et al., 2011, J. Biol. Chem. 286:22510-22520). CtXyl5A specifically cleaves highly arabinosylated xylan generating oligosaccharide products with Ara-1→3-Xyl at the reducing end, a structure that has been proposed as a specificity determinant for the enzyme. We interpret the lack of cleavage of YS2 by CtXyl5A to indicate that YS2 does not contain appreciable amounts of O-3-linked Xyl. This conclusion is in agreement with the YS2 sugar linkage data showing that 26% of Xyl in YS2 is O-2 substituted (Table 8). Taken together these results indicate that the xylan in YS2 is partially O-2 arabinosylated.

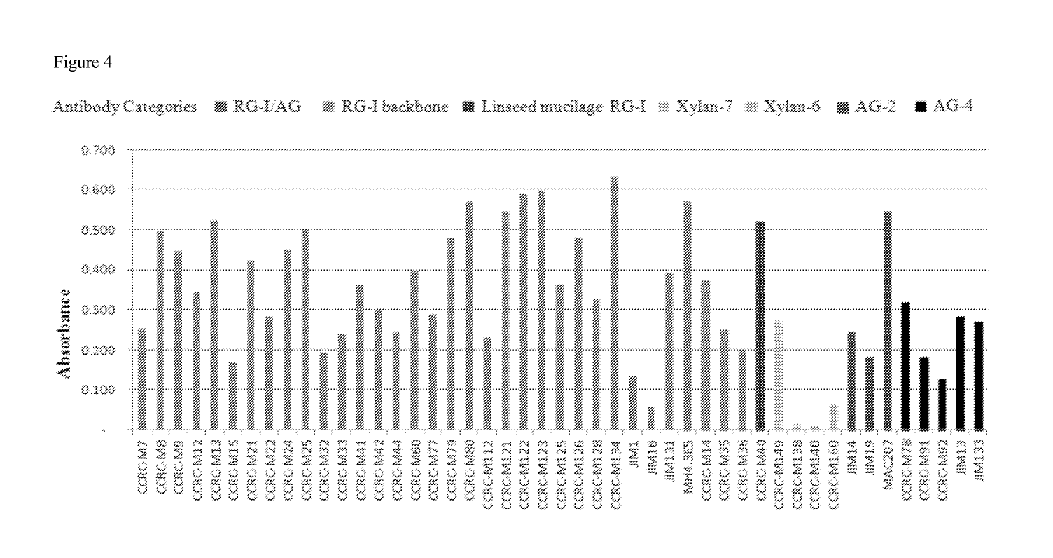

To further study the nature of the pectin and arabinoxylan regions in the AGP structure, YS2 was probed for cross-reactivity with 47 antibodies raised against RG-I, xylan, and AG (Pattathil et al., 2010, Plant Physiol. 153:514-525). Reactivity of YS2 against antibody classes AG-2, AG-4, RG-I/AG, RG-1 backbone, linseed mucilage RG-I, and xylan 7 (Pattathil et al., 2010, Plant Physiol. 153:514-525) confirmed the presence of RG-I, xylan, and AG glycan epitopes in YS2 (see FIG. 4). Among the four selected anti-xylan antibodies, only CCRC-M149 showed strong reaction with YS2. We propose that reaction with the other xylan-reactive antibodies may have been limited due to the high level of arabinosylation and acetylation (see below) of xylan in YS2.

The results led us to propose that YS was an AGP proteoglycan with two arabinoxylan containing regions, one attached to a pectin moiety and one not. To test this we first asked how the pectin-arabinoxylan domain and the separate arabinoxylan domain (henceforth referred to as the arabinoxylan) domain) were linked to the AGP. We postulated that the pectin-arabinoxylan domain and the arabinoxylan) domain were linked either to the AG domain or to the polypeptide portion of the AGP.

### Hyp Residues are the Attachment Sites for the Glycan to the Protein Core in YS and Naming of YS as Arabinoxylan-Pectin-Arabinogalactan-Protein 1 (APAP1)

To determine if the glycan in YS1 and YS2 was O-glycosylated to Hyp, or to Ser/Thr, or to both types of amino acids in the AGP protein core, a β-elimination reaction known to remove sugars O-linked to Ser/Thr was used (Kieliszewski et al., 1992, Plant Physiol. 99:538-547). AGPs are known to contain long AG glycans and short oligoarabinosides attached to Hyp residues (Kieliszewski, 2001, Phytochemistry 57:319-323), and these Hyp-O-linked glycans/oligosaccharides are stable to β-elimination (Lamport and Miller, 1971, Plant Physiol. 48:454-456; Tan et al., 2004, J. Biol. Chem. 279:13156-13165). β-Elimination of YS1 resulted in only a small decrease in the mole percentage of Gal (See Table 3) and a total sugar weight decrease from 53.3% (intact) to 50.9%. These results agree with prior reports showing that Ser/Thr residues of AGPs are usually glycosylated with single Gal residues. The results further suggested that the glycan in YS 1 was attached via O-glycosylation to Hyp in the AGP57C polypeptide (Kieliszewski, 2001, Phytochemistry 57:319-323).

TABLE 3
Sugar composition (mole percentage) of APAP1 YS1 and β-eliminated
YS1. Neutral sugars were analyzed via the alditol acetate
method, while uronic acid residues were estimated by colorimetric
method as described in the methods section. Sugar residues
in YS1 and β-eliminated YS1 account for 53.3% and 50.9%
of total weight, respectively.

| Sugar Residues | | |
|---|---|---|
| (Mol %) | YS1 | β-eliminated YS1 |
| Rha | 4.3 | 5.6 |
| Ara | 31.5 | 36.3 |
| Xyl | 28.8 | 29.5 |
| Uronic acid | 17.6 | 17.5 |
| Gal | 17.8 | 11.1 |

To confirm that the AG, RG-I, and arabinoxylan glycan domains were indeed directly or indirectly O-linked to Hyp, the glycosides attached to Hyp (Hyp-O-glycosides) were released from YS1 and YS2 by treatment with mild base, separated, and analyzed (Tan et al., 2004, J. Biol. Chem. 279:13156-13165; 2010, J. Biol. Chem. 285:24575-24583). The Hyp-O-glycoside profile revealed that about 70% of Hyp residues in both YS 1 and YS2 are attachment sites for large polysaccharides. The remaining Hyp residues are in part glycosylated with oligoarabinosides (˜11-13%), with single Ara residues (5-7%), or are present as free Hyp (nonglycosylated) (˜11%) (See Table 4). These results confirmed that YS 1 and YS2 are AGPs with both long glycan and oligoarabinose substitutions at Hyp residues.

TABLE 4
Hyp-glycoside profile of YS1 and YS2. Mole percentage
of Hyp as Hyp-glycosides (Hyp-polysaccharides or
Hyp-Ara₍ₙ₎) or free Hyp in total amount of Hyp residues.

| Hyp-glycoside | YS1 | YS2 |
|---|---|---|
| Hyp- | 69.7% | 71.8% |
| polysaccharides | | |
| Hyp-Ara₄ | Trace | Trace |
| Hyp-Ara₃ | 6.1% | Trace |
| Hyp-Ara₂ | 6.6% | 11.3% |
| Hyp-Ara | 7.1% | 5.3% |
| Hyp | 10.5% | 11.6% |

Size exclusion chromatography of the Hyp-O-polysaccharides released from YS 1 and YS2 by base hydrolysis, combined with colorimetric analyses revealed that pentose residues and Hyp residues co-chromatographed (FIGS. 2C and 1D), as expected for glycans covalently attached to Hyp (Tan et al., 2004, J. Biol. Chem. 279:13156-13165). Furthermore, sugar composition analyses of YS1 and YS2 Hyp-O-polysaccharide fractions representing large and small Hyp-O-polysaccharides, fractions 22 and 50, respectively, (FIGS. 2C and 1D; Table 5) showed that the large Hyp-O-polysaccharides still contained the characteristic Xyl, uronic acid, and Rha as well as Gal and Ara. Importantly, based on 2-AB reducing end labeling tests, no reducing end sugar residues were detected in representative YS2 Hyp-O-polysaccharide fraction 24 (FIG. 2D). Specifically, 2AB-treated Hyp-O-polysaccharides had a maximum UV absorbance of only ˜220 nm while 2-AB-labeled control polygalacturonic acid or RG-I oligomers displayed maximum UV absorbances at 254 nm. These results confirmed the direct or indirect covalent attachment of the arabinoxylan, RG-I, and AG glycan domains to Hyp residues in AGP57C.

TABLE 5
Sugar composition of YS1 and YS2 Hyp-O-polysaccharides. Total
Hyp-O-polysaccharides from each sample were fractionated
on an S-75 size exclusion chromatography analytical column.
Sixty μg of each selected fraction were used for neutral
sugar analysis or uronic acid colorimetric analysis. Fraction
22 and 50 represent a large- and a small-sized Hyp-poly/oligosaccharide
from each sample, respectively.

| | YS1 Hyp- | YS1 Hyp- | YS2 Hyp- | YS2 Hyp- |
|---|---|---|---|---|
| Sugar | polysac- | polysac- | polysac- | polysac- |
| Residue | charide | charide | charide | charide |
| (Mol %) | Fraction 22 | Fraction 50 | Fraction 22 | Fraction 50 |
| Rha | 2.2 | 0 | 6.1 | 0 |
| Ara | 32.6 | 44.5 | 37.5 | 76.8 |
| Xyl | 24.0 | 3.8 | 37.0 | 10.6 |
| Gal | 27.3 | 46.2 | 10.4 | 7.0 |
| Uronic acid | 13.9 | 5.6 | 9.1 | 5.6 |

The combined results provided compelling evidence that AGP57C, encoded by Arabidopsis gene At3g45230, is the core of a proteoglycan with covalently attached AG, arabinoxylan, and pectin domains. To distinguish the AGP core protein from the highly glycosylated proteoglycan structure identified here, from this point forward we refer to the AGP protein core as AGP57C and to the full proteoglycan structure as APAP1 (Arabinoxylan-Pectin-Arabinogalactan-Protein 1).

### the AG Glycans in YS 1 and YS2 are Attached Directly to Hyp in the Protein Core

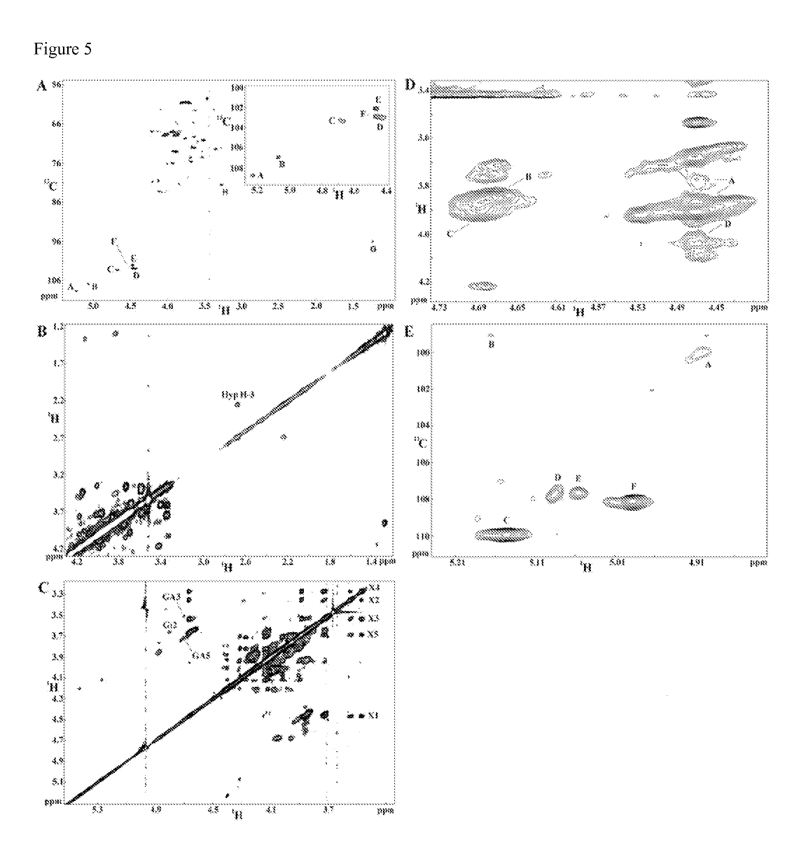

We next sought to determine which of the three glycan domain(s) in YS, i.e. the AG, pectin, and/or arabinoxylan domains, were directly attached to Hyp residues in the protein core of APAP1. To achieve this, YS1 Hyp-O-polysaccharide fractions 26 and 27 (named as YS1-HP2627, see FIG. 2C) were pooled for NMR analysis because of their relative abundance. Sugar analyses of YS1-HP2627 revealed a Gal:Ara:Xyl:GalA:Rha molar ratio of 14:10.2:3.1:1.5:1. The mild base treatment used to generate YS1-HP2627 almost completely β-eliminated Rha and GalA residues, thus simplifying its structure and the linkages between Hyp, AG and Xyl residues. The Heteronuclear Single Quantum Coherence (HSQC) spectrum of YS1-HP2627 (FIG. 5A) resembled those reported for Hyp-O-arabinogalactan (Hyp-O-AG) polysaccharides (Tan et al., 2004, J. Biol. Chem. 279:13156-13165; 2010, J. Biol. Chem. 285:24575-24583), except for the additional signals attributed to 1,4-β-D-Xylp, suggesting the presence of Hyp-O-AG structure in this Hyp-O-polysaccharide fraction. Furthermore, the identification of Hyp protons from the ¹H Homonuclear Correlation Spectroscopy (COSY) spectrum (FIG. 5B) and the unique Gal residue O-linked to Hyp from the Total Correlation Spectroscopy (TOCSY) spectrum (FIG. 5C) established the Hyp-O-Gal linkage in YS1-HP2627 (Tan et al., 2004, J. Biol. Chem. 279:13156-13165; 2010, J. Biol. Chem. 285:24575-24583). These results indicated that it was the AG domain that was directly linked to the protein core in YS 1. The Hyp-O-Gal linkage was also identified in Hyp-O-polysaccharide fraction YS2-HP3132 from YS2 (fractions 31 and 32 in FIG. 2D). YS2-HP3132 had a Gal:Ara:Xyl:uronic acid:Rha molar ratio of 3:2:7:1.4:1, indicating that this particular YS2 Hyp-O-polysaccharide fraction was more highly xylosylated than that from YS 1.

The type II AG linkages in YS1-HP2627 were further supported by NOE assignments in the Nuclear Overhauser Effect Spectroscopy (NOESY) spectrum (FIG. 5D) including assignments for Gal residues (Gal_{bb}) in the galactan backbone [β-D-Gal_{bb}-1→3-β-D-Gal_{bb} and β-D-Gal_{bb}-1→6-β-D-Gal_{bb}] and side chain Gal (Gals) residues [β-D-Gal_{sc}-1→6-β-D-Gal_{bb}]. These data showed that the AG domain in YS 1 and YS2 was linked to the protein core in the same way as in common AGPs (Kieliszewski, 2001, Phytochemistry 57:319-323). Taken together the above results indicated that the AG domain in APAP1 was linked to Hyp in the polypeptide core and that the pectinarabinoxylan and arabinoxylan1 domains were linked in some manner to the AG domain. In order to identify the precise covalent linkages of the pectin-arabinoxylan and arabinoxylan1 domains to the AG domain, we carried out a series of NMR and MS-based analyses.

### Pectin is Attached to the Rhamnosylglucuronosyl Side Chain of AG

AGPs isolated from many plant materials have been shown to have t-Rha-1→4-β-D-GlcA-1→6-β-D-Gal_{sc} as one of the bifurcate side chains of the AG glycan domain (Defaye and Wong, 1986, Carbohydr. Res. 150:221-231; Gane et al., 1995, Carbohydr Res. 277:67-85; Tan et al., 2004, J. Biol. Chem. 279:13156-13165). A GlcA residue was detected in YS2 by the TMS composition method, but was not detected in YS1 due to its lower abundance. However, signals characteristic for 4-β-D-GlcAp were obtained in HSQC and TOCSY spectra of YS1-HP2627 (FIGS. 5A and 3C) which yielded ¹H/¹³C signals of ˜4.51/˜102.1 (H/C-1), 3.713/75.317 (H/C-5) and 3.501 (H-3) ppm (Tan et al., 2004, J. Biol. Chem. 279:13156-13165). Furthermore, NOEs between GlcA H-1 and the AG side chain Gal (Gal_{sc}) H-6 (3.92 ppm) in YS1 also supported the GlcA-1→6-Gals linkage (FIG. 5D) in YS 1.

A Heteronuclear Multiple Quantum Coherence (HMQC) spectrum of YS1-HP2627 (FIG. 5E) revealed anomeric ¹H/¹³C signals at 4.903/100.077 ppm, typical 2-α-L-Rha signals for RG-I (Zheng and Mort, 2008, Carbohydr. Res. 343:1041-1049), suggesting the residual Rha on the typical t-Rha-1→4-β-D-GlcA-1→6-β-D-Gal_{sc} AG side chain was O-2-substituted in YS1-HP2627. In addition, anomeric signals (₁H/¹³C 5.167/99.115 ppm) for α-D-GalA residues were also found in the HMQC spectrum of YS1 (See Table 6). Taken together, the identification of the AG side chain 4-β-D-GlcA and characteristic 2-α-L-Rha and α-D-GalA of pectin, as well as the establishment of the Xyl to Ara linkage in YS1-HP2627 as shown below, are consistent with the existence in YS1-HP2627 of the following structural unit as a side chain of the AG: α-D-GalA-1→2-α-L-Rha-1→4-β-D-GlcA-1→6-β-D-Gal_{sc}. It also indicates that the non-reducing end Rha of the AG side chain may serve as the attachment site of pectin.

TABLE 6
Chemical shift assignments of YS1-HP2627. The chemical shifts were assigned based on
HSQC and TOCSY spectra which were collected at 25° C. on a Varian VNMR 800 instrument,
and based on published data (Tan et al., 2004, J. Biol. Chem. 279: 13156-13165).

| | C-1/H-1 | C-2/H-2 | C-3/H-3 | C-4/H-4 | C-5/H-5 | C-6/H-6 |
|---|---|---|---|---|---|---|
| Residues | ppm | | | | | |
| 4-β-D-Xylp | ~102/~4.46 | 72.408/3.344 | 74.620/3.532 | 81.607/3.269 | 62.385/3.684 | — |
| 3-α-L-Araf | 108.737/5.223 | 81.714/4.220 | 83.508/4.026 | 83.258/4.108 | 60.645/3.788 | — |
| 5-α-L-Araf | 108.737/5.223 | 80.754/4.206 | 76.195/4.002 | 83.673/4.115 | 66.273/3.863, | — |
| | | | | | 3.791 | |
| t-α-L-Araf | 106.902/5.068 | 80.337/4.118 | 76.019/3.931 | 83.446/4.074 | 60.645/3.814, | — |
| | | | | | 3.697 | |
| β-D-Gal_{bb} | 103.207/4.688 | 69.695/3.763 | 81.255/3.859 | 67.859/4.125 | 73.075/3.898 | 68.8/~4.0, |
| | | | | | | 3.9 |
| β-D-Gal_{bb} | 103.359/4.679 | 69.765/3.763 | 81.380/3.843 | 67.859/4.125 | 73.075/3.898 | 68.8/~4.0, |
| | | | | | | 3.9 |
| β-D-Gal_{sc} | 102.851/4.469 | 69.320/3.666 | 79.711/3.721 | n.a. | 73.034/3.948 | 68.8/~4.0, |
| | | | | | | 3.9 |
| β-D-Gal_{sc} | 102.923/4.432 | 69.278/3.642 | 79.711/3.721 | n.a. | n.a. | n.a. |
| β-D-Gal_{sc} | 103.000/4.424 | 69.278/3.629 | 79.711/3.721 | n.a. | n.a. | n.a. |
| α-L-Rhap | 100.077/4.903* | n.a. | ~69.7/3.784 | n.a. | n.a. | 16.074/1.235 |
| β-D-GlcAp | 102.154/4.513 | n.a. | n.a. | 80.462/3.501 | 75.317/3.713 | n.a. |
| α-D-GalAp | 99.115/5.167* | n.a. | n.a. | n.a. | n.a. | n.a. |

*Data from weak signals on an HMQC spectrum collected at 30° C.
n.a.: not analyzed due to signal overlapping.
Gal_{bb}: Gal in the 1→3-galactan backbone
Gal_{sc}: Gal in the AG side chain

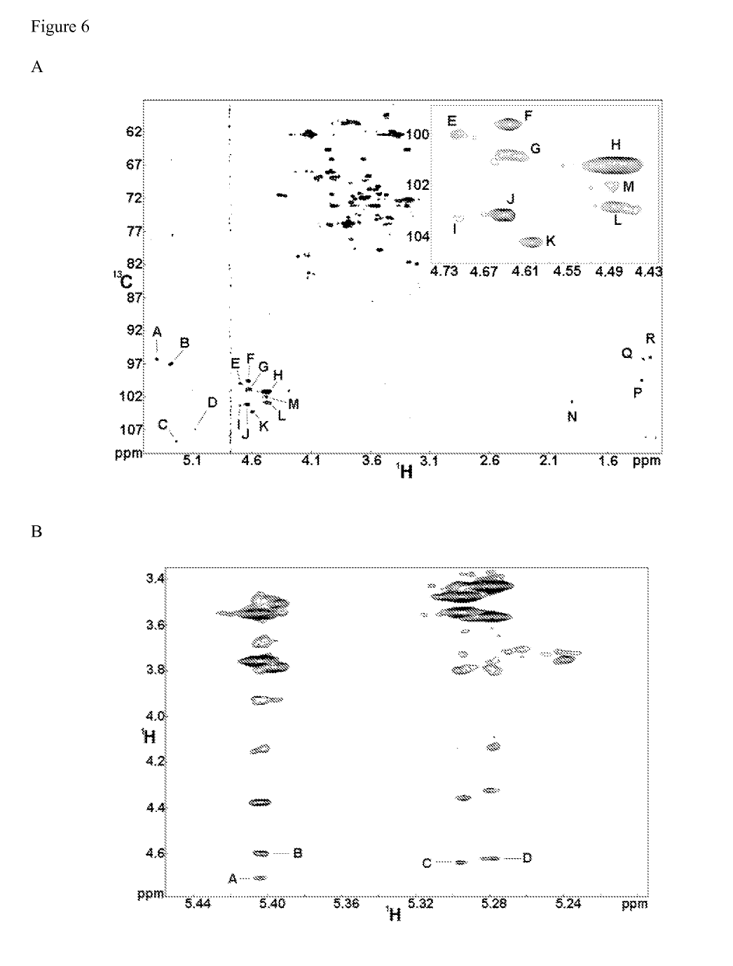

The base treatment that generated YS2 Hyp-O-polysaccharide fractions 26 and 27 (YS2-HP2627) did not completely β-eliminate the pectic 2(,4)-α-Rha and 4-α-GalA residues and even some acetyl groups remained intact after base treatment, as evidenced in the HSQC spectrum (see FIG. 6A and Table 7). 4-β-GlcA ₁H/¹³C signals were also identified in the HSQC spectrum. Because the AG was the glycan domain attached to the polypeptide as demonstrated above and there was no t-Rha in YS2-HP2627, these results provided further evidence for the occurrence of -4-α-D-GalA-1→2-α-L-Rha-1→4-β-D-GlcA-1→6-β-D-Gal_{sc} structures in YS. Furthermore, the 1:1 ratio of anomeric protons between α-LRha and α-D-GalA in YS2-HP2627 indicated that the remaining pectic fragment on YS2-HP2627 was RG-I. These results support the conclusion that the rhamnosyl residue in the rhamnosylglucuronsyl side chain of AG serves as an attachment site for RG-I.

TABLE 7
Chemical shift assignment of YS2-HP2627. The chemical shifts were assigned based
on HSQC and TOCSY spectra collected at 25° C. on a Varian VNMRS 800 instrument.

| | C-1/H-1 | C-2/H-2 | C-3/H-3 | C-4/H-4 | C-5/H-5 | C-6/H-6 |
|---|---|---|---|---|---|---|
| Residues | ppm | | | | | |
| 4-α-D-GalAp | 96.318/5.402 | 70.220/3.530 | 69.687/3.758 | 76.046/3.929 | 71.528/4.374 | n.a. |
| 4-α-D-GalAp | 96.318/5.397 | n.a. | n.a. | 75.883/3.965 | 71.624/4.323 | n.a. |
| 2,4-α-L-Rhap | 97.103/5.296 | n.a. | n.a. | n.a. | 72.622/3.788 | 16.109/1.227 |
| 2-α-L-Rhap | 96.980/5.276 | n.a. | 72.089/3.730 | n.a./3.545 | 67.976/4.098 | 16.269/1.293 |
| 3(5)-α-L-Araf | 108.711/5.239 | 80.779/4.213 | 83.997/4.129 | 83.468/4.078 | n.a. | — |
| t-α-L-Araf | 106.877/5.080 | 80.664/4.127 | ~76/~3.94 | 83.224/4.122 | n.a. | — |
| β-D-Gal_{bb} | 103.170/4.638 | 69.163/3.617 | n.a. | n.a. | 72.943/3.955 | ~68.9/~4.0, |
| | | | | | | ~3.92 |
| β-D-Gal_{sc} | ~102.9/~4.46 | 69.285/3.651 | n.a. | n.a. | n.a. | ~68.9/~4.0, |
| | | | | | | ~3.92 |
| 4-β-D-Gal | 104.255/4.595 | 70.586/3.613 | 71.967/3.672 | 76.113/3.937 | n.a. | 60.426/~3.76 |
| 4-β-D-GlcA | 102.098/4.511 | 73.105/3.346 | 74.975/3.517 | 79.729/~3.58 | 74.365/3.723 | n.a. |
| 3-OAc-4-Xylp | 99.980/4.705 | 74.650/3.695 | 77.547/5.270 | 76.275/3.805 | 62.295/3.551 | 3-OAc: |
| | | | | | | 22.694/1.911 |
| 2,4-β-D-Xylp | 99.690/4.632 | 81.558/3.296 | 74.853/3.522 | 76.275/3.813 | 62.376/4.108, | — |
| | | | | | 3.414 | |
| 4-β-D-Xylp | 100.832/4.619 | n.a. | 73.105/3.615 | ~76.28/3.794 | 62.376/~4.1, | — |
| | | | | | 3.434 | |
| 4-β-D-Xylp | 101.169/4.472 | n.a. | 3.694 | n.a. | n.a. | — |

n.a.: not analyzed due to signal overlapping.
Gal_{bb}: Gal in the 1→3-galactan backbone
Gal_{sc}: Gal in the AG side chain

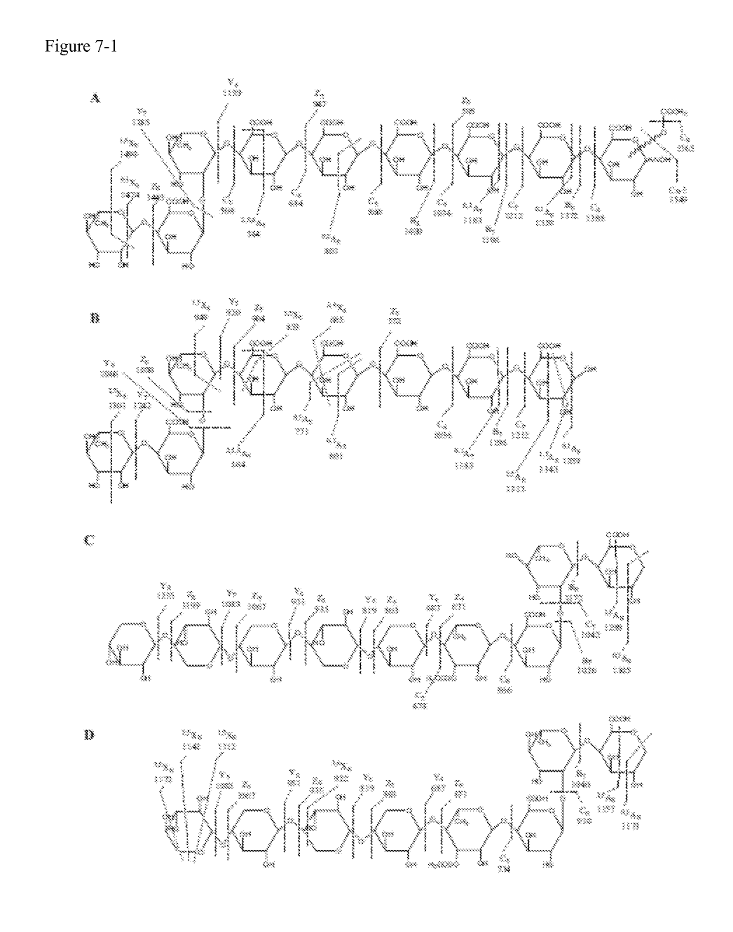

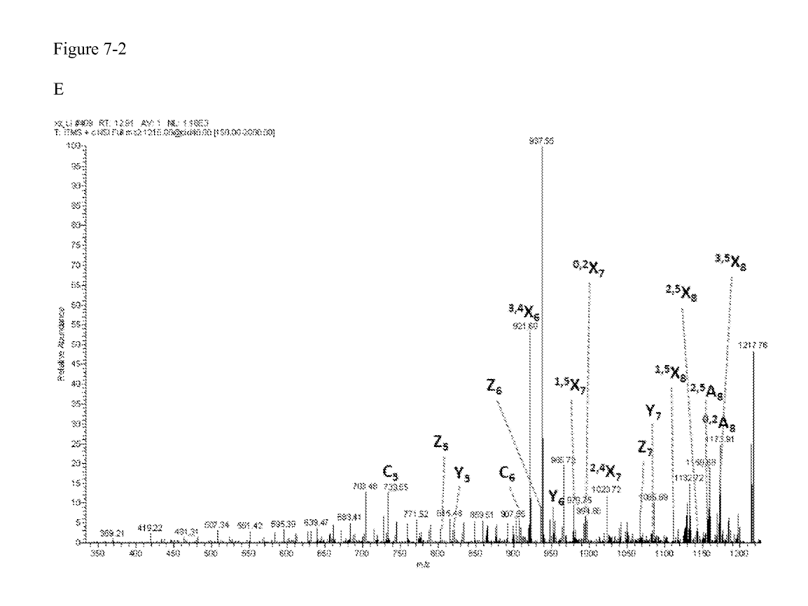

The higher percentages of GalA than Rha in YS suggests the existence of homogalacturonan in the pectin domain of APAP1 (Table 8). Oligosaccharides released from YS2 by treatment with RGH and analyzed by CID-MS/MS, yielded fragments that indicated the presence of RG-I and HG domains in YS2. The MS₂ fragmentation of parent ions at m/z 1608 and 1389 yielded a series of B, C, Y, and Z ions (FIGS. 7A and 5B). Together with the sugar linkage analysis of YS2 and the hydrolytic features of RGH, these MS₂ fragments are consistent with the oligomer structures Rha-GalA-Rha-GalA-GalA₄-GalA-[-O—COCH₃] ([M+H+Na]₊, m/z 1608) and Rha-GalA-Rha-GalA-(GalA)₃-GalA ([M+H+Na]₊, m/z 1389), respectively. The results indicate that at least short homogalacturonan regions are part of the pectin domain in APAP1 and that they form a continuous pectin backbone with RG-I in APAP1.

TABLE 8
Glycosyl composition^{a} and linkage^{b} analysis of sugar residues in YS1, YS2, and Ara101P.
All numbers are mole percentages. The high percentage of t-Ara in YS2 resulted from the
17% of Hyp residues in APAP1 branched with Ara₂ or Ara residues, as well as the t-Ara
residues that serve as side chains of arabinoxylan and arabinogalactan.

| Glycosyl | | | | Glycosyl | | | |
|---|---|---|---|---|---|---|---|
| Residue^{a} | YS1^{a,c} | YS2^{a,d} | Ara101P^{a,e} | Linkage | YS1^{b,c} | YS2^{b,d} | Ara101P^{b,e} |
| Ara | 37.7 | 39.4 | 64.8 | t-Araf | 3.0 | 22.0 | 15.7 |
| Xyl | 23.4 | 39.2 | Trace | 3-Araf | 0.8 | 1.9 | 0.4 |
| GalA | 11.0 | 6.6 | 11.4 | 5-Araf | 10.4 | 8.2 | 8.4 |
| Rha | 5.7 | 5.6 | 10.4 | 3,5-Araf | 5.5 | 2.3 | 1.9 |
| Gal | 19.9 | 6.8 | 13.4 | 2,5-Araf | — | 3.6 | 4.2 |
| GlcA | — | 1.1 | — | t-Arap | — | 5.1 | 0.4 |
| Fuc | — | 0.3 | — | 2,3,4-Arap | 4.8 | 3.3 | 4.3 |
| Glc | — | 1.0 | — | 2,4-Arap | 5.0 | — | — |
| O-methyl | 1.7 | — | — | t-Xylp | — | 4.2 | — |
| sugar | | | | | | | |
| Unknown | 0.6 | — | — | 4-Xylp | 5.7 | 16.9 | — |
| sugar | | | | | | | |
| | | | | 2,4-Xylp | 4.2 | 7.5 | — |
| | | | | 4-GalAp | 13.4 | 6.3 | 8.2^{f} |
| | | | | 4,6-GalAp | — | — | 10.7 |
| | | | | t-Rhap | — | — | 0.2 |
| | | | | 2-Rhap | 6.6 | 3.1 | 15.3 |
| | | | | 4-Rhap | — | — | 0.3 |
| | | | | 2,3-Rhap | 1.2 | — | 0.3 |
| | | | | 2,4-Rhap | 4.3 | 1.2 | 12.3 |
| | | | | 2,3,4-Rhap | 0.8 | — | — |
| | | | | t-Galp | 2.2 | 2.3 | 9.4 |
| | | | | 3-Galp | 4.9 | 3.9 | 2.0 |
| | | | | 4-Galp | 3.3 | 1.9 | ^{f} |
| | | | | 6-Galp | 10.0 | 1.6 | 2.6 |
| | | | | 3,6-Galp | 13.9 | 3.2 | 2.2 |
| | | | | 3,4,6-Galp | — | — | 0.2 |
| | | | | t-GlcAp | — | — | 0.3 |
| | | | | 4-GlcAp | — | 1.6 | 0.2 |
| | | | | 4-Manp | — | — | 0.3 |
| | | | | 3,4-Glcp | — | — | 0.4 |

^{a}Glycosyl residue composition by GC-MS analysis of trimethylsilyl derivatives. Data are average of two separate analyses by CCRC Analytical Services.
^{b}Sugars were converted to partially methylated alditol acetates and analyzed by GC-MS.
^{c}Total average weight % carbohydrate of YS1 was 63.2%
^{d}Total average weight % carbohydrate of YS2 was 97.7%
^{e}Total average weight % carbohydrate of Ara101P was 78.0%
^{f}Total molar percentage of 4-GalAp and 4-Galp was 8.2

### Arabinoxylan1 is 1→5 O-Linked to an Arabinosyl Residue in the AG Domain of APAP1

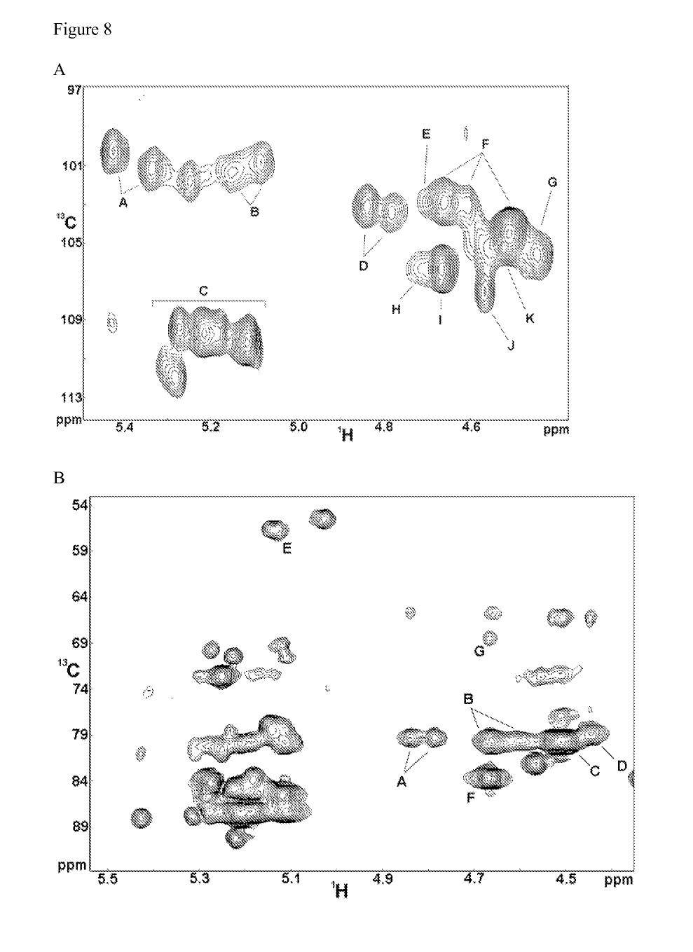

Four types of β-D-Xylp residues were identified in the NMR spectra of YS2 (See FIG. 8), including 3-OAc-(2,4)-β-Xylp, 3-OAc-(4)-β-Xylp, 2,4-β-D-Xylp, and 4-β-DXylp (see Table 9). The -β-Xylp-1→4-P-Xylp-linkages in the arabinoxylan domain of YS2 were confirmed by the HMBC connections. Furthermore, the HMBC connection of 2,4-P-Xylp H-1 to Araf C-5 showed that some of the 2,4-P-Xylp residues were 1→5 attached to [symbol] -L-Araf in YS2. These results indicated that arabinoxylan was either attached to Ara residues present in the AG domain or to 1,5-arabinan in a side chain of RG-I.

TABLE 9
Chemical shift assignments of YS2. The chemical shifts were assigned based on HSQC,
COSY, TOCSY, and HMBC spectra, collected at 55° C. on a Bruker-DRX800 instrument.

| | C-1/H-1 | C-2/H-2 | C-3/H-3 | C-4/H-4 | C-5/H-5 | C-6/H-6 |
|---|---|---|---|---|---|---|
| Residues | ppm | | | | | |
| α-D-GalAp | 100.196/5.421 | ~71/3.753 | n.a. | n.a./~3.7 | n.a. | n.a. |
| α-D-GalAp | 101.184/5.334 | ~71/3.617 | n.a. | n.a./~3.7 | n.a. | n.a. |
| 2-α-L-Rhap | 101.326/5.148 | 79.298/3.990 | n.a. | 75.028/n.a. | 72.677/3.666 | 19.317/1.287 |
| 2,4-α-L-Rhap | 100.831/5.084 | 79.148/3.939 | n.a. | 83.794/3.74 | 71.156/3.910 | 19.601/1.358 |
| α-L-Araf | 111.914/5.282 | ~84.5/4.251 | n.a. | n.a. | n.a. | — |
| α-L-Araf | 109.373/5.270 | ~83.9/4.334 | n.a. | n.a. | n.a. | — |
| α-L-Araf | 109.655/5.214 | ~83.9/4.182 | n.a. | n.a. | n.a. | — |
| α-L-Araf | 109.726/5.200 | ~83.9/4.182 | n.a. | n.a. | n.a. | — |
| α-L-Araf | 109.655/5.181 | ~83.9/4.182 | n.a. | n.a. | n.a. | — |
| α-L-Araf | 110.079/5.113 | ~83.9/4.184 | n.a. | n.a. | n.a. | — |
| 3-OAc-2,4-Xyl | 103.099/4.838 | 82.453/3.801 | 76.512/5.138 | ~79/3.777 | 65.719/3.971 | 3-OAc: |
| | | | | | | 23.469/2.204 |
| 3-OAc-2,4-Xyl | 103.435/4.781 | 82.453/3.751 | 76.716/5.120 | ~79/3.736 | 66.248/3.984 | 3-OAc: |
| | | | | | | 23.469/2.204 |
| 3-OAc-4-Xyl | 102.973/4.689 | n.a./3.661 | 76.933/5.118 | ~79/3.835 | n.a. | 3-OAc: |
| | | | | | | 23.469/2.204 |
| 2,4-Xyl | 102.906/~4.66 | 84.055/3.346 | 74.105/3.492 | ~79/3.811 | 65.719/3.996 | — |
| 2,4-Xyl | 105.137/4.564 | 84.110/~3.48 | n.a. | ~79/3.773 | ~65.8/3.975 | — |
| 2,4-Xyl | 104.530/4.496 | 84.055/3.339 | ~74.5/3.475 | ~79/n.a. | 66.286/3.924 | — |
| 4-Xyl | 105.618/4.443 | 75.506/3.275 | ~75.0/3.570 | ~79/3.777 | 66.286/4.088, | — |
| | | | | | 3.415 | |
| 3,6-β-D-Gal_{bb} | 106.267/4.71-4.66 | 69.180/3.684 | ~83.8/3.886 | n.a. | n.a./3.737 | n.a. |
| 3,6-β-D-Gal_{sc} | 107.467/4.564 | n.a./3.676 | ~82.2/n.a. | n.a. | n.a. | n.a. |
| 4-β-D-Gal | 104.502/4.509 | n.a./3.623 | n.a. | n.a. | n.a. | n.a. |

n.a.: not analyzed due to signal overlapping.
Gal_{bb}: Gal in the 1→3-galactan backbone
Gal_{sc}: Gal in the AG side chain

Further evidence for the attachment of arabinoxylan to the AG domain was provided by NOEs between β-D-Xylp H-1 and unique α-L-Araf H-5 of YS1-HP2627 in the NOESY spectrum (FIG. 5D) (Tan et al., 2004, J. Biol. Chem. 279:13156-13165). The ¹³C/₁H signals of Xylp were assigned based on the TOCSY and the HSQC spectra (See FIG. 5 and Table 6). From the ¹³C signals, we conclude that the Xyl residues in YS1-HP2627 are 4-linked, because there were no 2,4-bisubstituted Xylp residues with two characteristic ¹³C chemical shifts at 80 and 78 ppm (Mazumder and York, 2010, Carbohydr. Res. 345:2183-2193). Because only AG and xylan domains were predominant in YS1-HP2627, the Xylp-1→5-α-L-Araf linkage indicates that the xylan oligomers were attached to the arabinosyl residues in the AG domain. Linkage of xylan to Ara in the AG domain in APAP1 explains why substantial amounts of Xyl remained in YS2 after RGH treatment, while RG-I attached xylose residues were removed as described above.

### The Pectin-Arabinoxylan Moiety in APAP1 Consists of Arabinoxylan 2 Attached to Rha in the RG-I Domain

The NOESY spectrum (See FIG. 6B) of YS2-HP2627 clearly showed unique NOEs between some Xyl H-1 (4.597, 4.621, and 4.638 ppm) and Rha/GalA H-1 (5.402 ppm), indicating a Xyl-O-Rha/or GalA linkage. Because only 2,4-Rha residues but not 2,4-GalA residues were detected in YS2, these NOEs suggested that some Xyl residues were attached to the Rha in the RG-I domain in YS2 through-4-β-D-Xylp-1→4-[-α-D-GalA-1→2-]-α-L-Rhap linkage. The Xyl to Rha linkages were further supported by CID-MS/MS analysis identifying fragment ions of oligosaccharides released from YS2 with RGH. The MS² fragmentation of parent ions at m/z 1348 and 1216 showed a range of Y and Z, B and C ions. These fragment ions were consistent with the following structures, Xylp-(Xylp)₃-Xylp-(AcO)Rha-GalA-Rha-GalA ([M−H₂O+2H]₊, m/z 1348) and Xylp-(Xylp)₂-Xylp-(AcO)Rha-GalA-Rha-GalA ([M−H₂O+2H]₊, m/z 1216) (FIGS. 7C, 7D, and 7E). These data establish the arabinoxylan (named arabinoxylan 2) to RG-I covalent attachment through the Xyl-1→4-Rha linkage, and explain why RGH released 44% of the Xyl from YS2 (see Table 2).

### Proposed Structure of APAP1

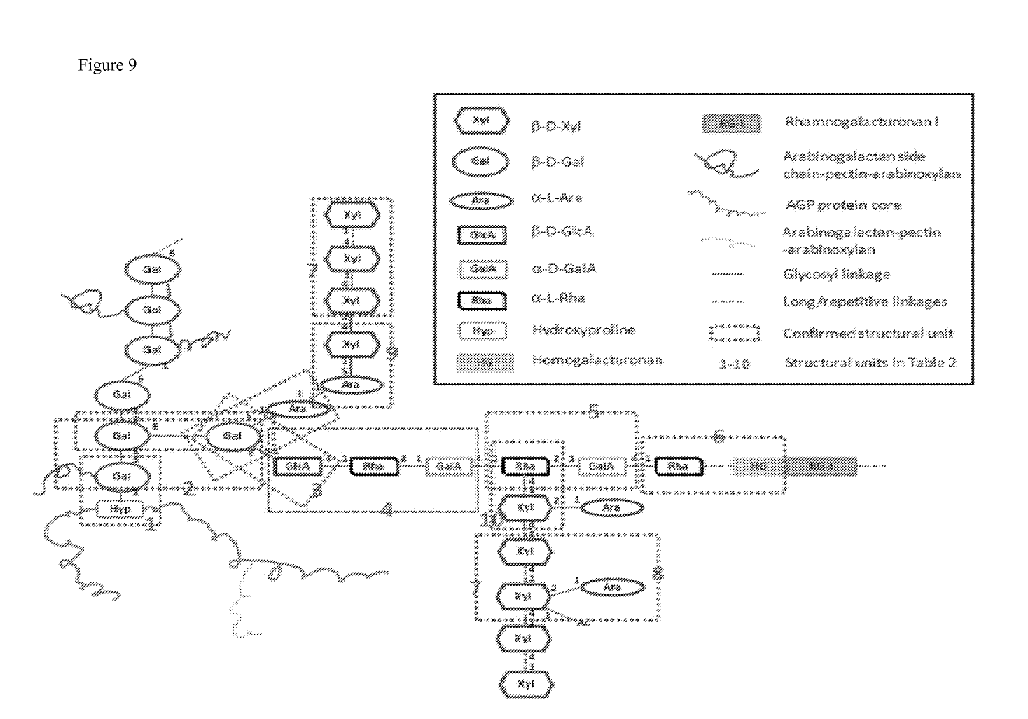

Based on the above results as summarized in Table 10, we propose the following structure for APAP1 (FIG. 9). APAP1 is built on an arabinogalactan protein core (encoded by At3g45230) to which are attached the classic type II AG polysaccharides. The pectin domain of APAP1 is composed of both RG-I and HG, with relatively small HG oligosaccharides flanked by RG-I oligomers on the pectin backbone. The range of lengths of the RG and HG domains is not known. The reducing end GalA residue of an RG-I/HG polysaccharide is linked to a non-reducing end Rha residue in a Rha-1→4-GlcA AG side chain. The data strongly indicate that a typical [→2-α-L-Rha-1→4-α-GalA-1→] disaccharide repeat RG-I backbone is extended from this. Arabinoxylan 1 is directly attached to a side chain Ara residue of the AG domain through a 1→5 linkage. Arabinoxylan 2 is linked to the O-4 of Rha in the RG-I domain of APAP1 (FIG. 9).

TABLE 10
Summary of NMR and mass spectrometry (MS) evidence for structural units in APAP1, all of which is consistent with
the proposed APAP1 structure shown in FIG. 4. Gal_{bb} represents Gal residues in the β-1→3-galactan backbone of the
AG domain. Gal_{sc} represents side chain β-Gal residues connected 1→6 to the β-1→3-galactan backbone.
Structural unit numbers listed in this table are the same as those marked in FIG. 4. YS1-HP2627, YS2-HP2627 and
YS2-HP3132 represent the Hyp-O-polysaccharide fractions 26 plus 27, and 31 plus 32, generated from YS1 and YS2,
respectively. TOCSY is total correlation spectroscopy; HSQC is heteronuclear single-quantum correlation spectroscopy;
HMQC is heteronuclear multiple-quantum correlation spectroscopy; HMBC is heteronuclear multiple-bond correlation
spectroscopy; NOESY is nuclear Overhauser effect spectroscopy; CID-MS/MS is collision-induced dissociation-MS/MS.

| | Structure Unit | Identified Structures | Techniques used |
|---|---|---|---|
| 1 | AG to Hyp | Hyp-O-Galp | TOCSY and HSQC spectra of |
| | linkage | | YS1-HP2627 and YS2-HP3132 |
| | | Hyp-O-AG | TOCSY and HSQC spectra of |
| | | | YS1-HP2627 and YS2-HP3132 |
| 2 | Type II AG | -β-D-Galp_{bb}-1→3-β-D-Galp_{bb}-1- | HMBC spectrum of YS2 and |
| | | | NOESY spectrum of YS1 HP-2627 |
| | | -β-D-Galp_{bb}-1→6-β-D-Galp_{bb}-l- | NOESY spectrum of YS1-HP2627 |
| | | -β-D-Galp_{sc}-1→6-β-D-Galp_{bb}-1- | NOESY spectrum of YS1-HP2627 |
| 3 | GlcA side chain | -β-D-GlcAp-1→6-β-D-Galp_{sc}-1- | NOESY spectra of YS1-HP2627 |
| | of AG | | and YS2-HP2627 |
| 4 | Pectin to AG | α-D-GalAp-1→-2-α-L-Rhap-1→4-β-D-GlcAp | HMQC spectrum of YS1-HP2627 |
| | GlcA linkage | | |
| 5 | RG-I linkage | -2-α-L-Rhap-1→4-α-D-GalAp-1- | HMBC spectrum of YS2 |
| 6 | HG-RG-I attachment | Rha-GalA-Rha-GalA-(GalA)₄-GaLA-(OAc)-OH | CID-MS/MS of RGH released |
| | | | oligomers from YS2 |
| | | Rha-GalA-Rha-GalA-(GalA)₃-GalAOH | CID-MS/MS of RGH released |
| | | | oligomers from YS2 |
| 7 | Different linkages | -4[α-L-Araf-1→2,AcO-3]-β-D-Xylp-1→4-β-D-Xylp-1- | HMBC spectrum of YS2 |
| | of xyloses in | -4[AcO-3]-β-D-Xylp-1→4-β-D-Xylp-1- | HMBC spectrum of YS2 |
| | arabinoxylan | -4-β-D-Xylp-1→4-β-D-Xylp-1- | HMBC spectrum of YS2 |
| | | Xylp₆, Xylp₅, Xylp₄ | CID-MS/MS of RGH released |
| | | | oligomers from YS2 |
| 8 | Arabinose to xylose | α-L-Araf-1→2-β-D-Xylp- | HMBC spectrum of YS2 and |
| | linkage in arabinoxylan | | NOESY spectrum of YS2-HP2627 |
| 9 | Arabinoxylan to AG | β-Xylp-1→5-α-L-Araf | HMBC spectrum of YS2 and |
| | arabinosyl side | | NOESY spectrum of YS1-HP2627 |
| | chain linkage | | |
| 10 | Arabinoxylan to RG-I | β-Xylp-1→4-α-L-Rhap | NOESY spectrum of YS2-HP2627 |
| | rhamnosyl | Xylp-(Xylp)3-Xylp-(AcO)Rha-GalA-Rha-GalA | CID-MS/MS of RGH released |
| | residue linkage | | oligomers from YS2 |
| | | Xylp-(Xylp)2-Xylp-(AcO)Rha-GalA-Rha-GalA | CID-MS/MS of RGH released |
| | | | oligomers from YS2 |
| 11 | β-1,4-galactan side | →4-β-D-Galp-1→4-α-(2-)-Rhap | NOESY spectrum of YS2-HP2627 |
| | chain of RG-I | | |

### Identification of APAP1-Like Proteoglycan in Cell Walls of Arabidopsis Suspension Culture Cells

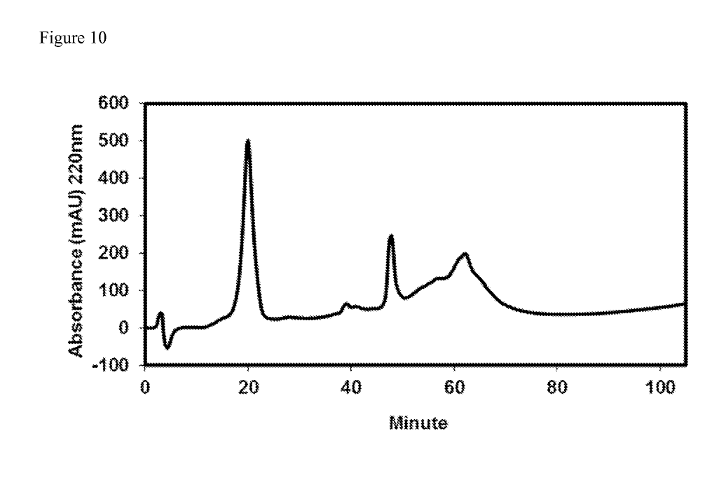

APAP1 was originally isolated from the medium surrounding Arabidopsis suspension cultured cells. To determine if APAP1 is present in plant cell walls, we analyzed an RG-1-enriched fraction prepared from an independent Arabidopsis suspension culture line (Guillaumie et al., 2003, Carbohydr. Res. 338:1951-1960). Cell walls were isolated, treated with mild base to remove methyl and acetyl esters, incubated with endo-polygalacturonase to obtain a pectin-enriched fraction, and then fractionated to yield an RG-1-enriched cell wall preparation as previously described (York et al., 1985, Methods Enzymol. 118:3-40). Further purification of this material using a method similar to the one used to isolate APAP1 (see methods) yielded a UV-220 nm absorbing protein fraction that was named Ara101P (See FIG. 10). Glycosyl residue composition and glycosyl linkage analyses showed that Ara101P consisted of sugar residues consistent with type II AG and RG-I structures (Table 8), suggesting that Ara101P was an APAP1-like proteoglycan. Furthermore, attachment of Ara101P to glutaraldehyde-activated magnetic amine beads, in a manner similar to that done for APAP1, followed by monosaccharide analysis of the Ara101P-beads (See Table 11), demonstrated that the AG and RG-I glycans were covalently attached to the polypeptide in Ara101P.

TABLE 11
Sugar composition of YS2 and Ara101P after covalent attachment
onto magnetic amine-beads. The monosaccharides released from
YS2-beads and Ara101P-beads via acid hydrolysis were analyzed
on the Dionex system as described in the methods.

| Glycosyl | | YS2 | | Ara101P |
|---|---|---|---|---|
| Residue | YS2- | before | Ara101P- | before |
| (mol %) | Beads | Beads^{a} | Beads | beads^{a} |
| Ara | 38.6 | 39.4 | 64.2 | 64.8 |
| Rha | 7.6 | 5.6 | 9.4 | 10.4 |
| Xyl | 36.5 | 39.2 | — | Trace |
| GalA | 8.4 | 6.6 | 10.3 | 11.4 |
| Gal | 8.9 | 6.8 | 16.1 | 13.4 |

^{a}from Table 8 in text; the 2.4% of other sugars of YS2 is not shown (see Table 8)

LC-MS/MS analysis of tryptic peptides from deglycosylated Ara101P identified a 22 amino acid sequence, SSOAOSODLADSOLIHASOOSK (O as Hyp) (SEQ ID NO:51). This peptide is in the N-terminal region of AtAGP57C, the same protein core of YS1 and YS2. These results provided independent evidence that the core protein of APAP1, AtAGP57C, was attached to pectin in an RG-1-enriched preparation released from cell walls of a different Arabidopsis suspension cell line than that used to identify APAP1. Importantly, these results demonstrate that AtAGP57C exists in cell walls in an APAP1-like form, i.e. RG-I/HG-AGP proteoglycan. Without diagnostic tools to probe intact APAP1 and APAP1-like molecules in intact cell walls, a difficult goal due to the very highly glycosylated nature of APAP1, it is not possible at this time to quantify the amount of such proteoglycans among all wall polymers, or to determine the proportion of APAP1-like proteoglycan that exists linked to pectin and xylan in the wall. The method used to isolate Ara101P selectively enriched for pectin-containing fractions, which are relatively easily released from the wall without harsh chemical treatments. Based on the amount of APAP1-like proteoglycan in the total RG-1-enriched pectin preparation, we calculate that more than 95% of the RG-I released from cell walls of Arabidopsis suspension cultured cells is covalently attached to AGPs.

### Identification of Two Arabidopsis Apap1 Homozygous Mutant Lines

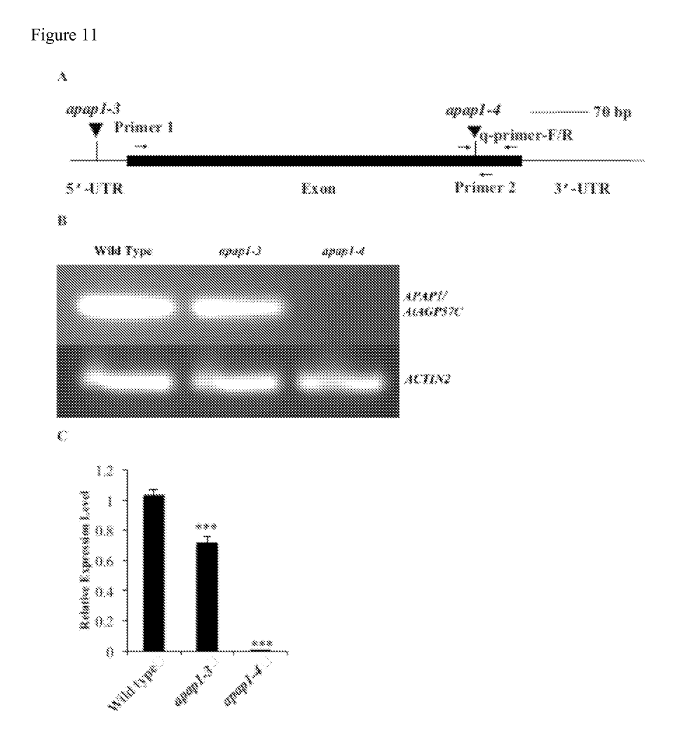

The above results indicated that APAP1 is a proteoglycan that connects an AGP to matrix polysaccharides in the wall. To determine the effect of reduced expression of the APAP1 core protein on cell wall structure in the plant, homozygous lines for two Arabidopsis apap1 homozygous SALK T-DNA insertion mutants (SALK_{—}070113c/apap1-3 and SALK_{—}002144/apap1-4) (see FIG. 11A) were identified. Semi-quantitative PCR of RNA prepared from leaves of wild type and two mutants showed reduced APAP1/AtAGP57C transcript in apap1-3 and complete absence of APAP1/AtAGP57C transcript in apap1-4 (see FIG. 11B). Further qPCR analysis of the RNA confirmed apap1-4 as a knockout line and apap1-3 as a knockdown line with a significant (˜31%) reduction in APAP1/AtAGP57C transcript (see FIG. 11C). The overall morphology of the apap1 mutants was comparable to wild type. However, growth measurements of 10 individual sets of wild type versus apap1-3 and apap1-4 mutants revealed that in 50% of the experiments there was a 5-25% significant increase in inflorescence stem height in the mutants compared to wild type and in the remaining experiments there was a non-significant trend for increased stem growth in the mutants.

### Mutation of APAP1 Effects Cell Wall Properties

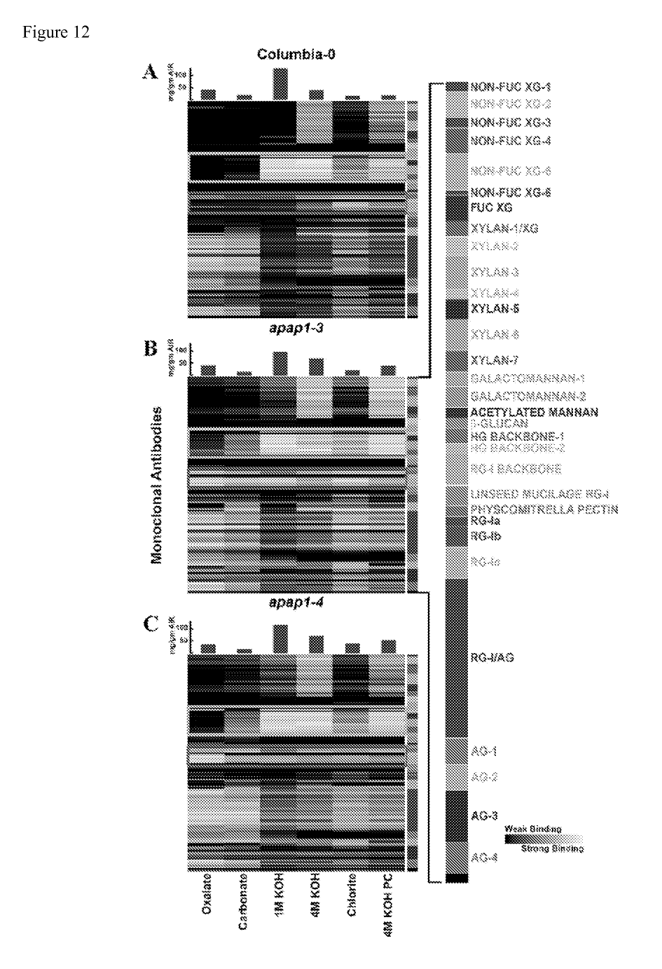

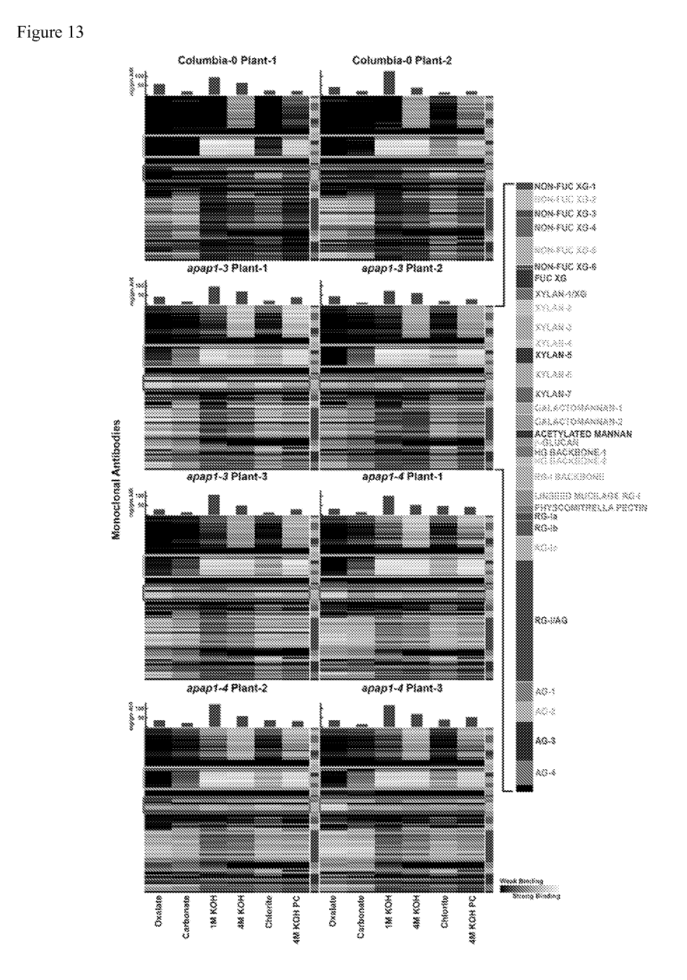

To determine if the apap1 mutation affected wall structure, glycome-profiling (Zhu et al., 2010) was used to compare extractable polysaccharides from Arabidopsis apap1 mutant versus wild type walls. We reasoned that if the apap1 mutation reduced covalent linkages between cell wall proteins, pectins and xylans, one or more components of the wall should be more easily extracted from the apap1 mutant walls. Because APAP1/AtAGP57C transcript is expressed in multiple Arabidopsis tissues including rosette leaves, siliques, seeds, flowers and the shoot apex region of inflorescence stem (eFP browser, available on the workl wide web at bar.utoronto.ca/efp/cgi-bin/efpWeb.cgi), cell walls from both whole aerial tissues and from stems were analyzed. Cell walls from aerial tissues of 8-weekold apap1-3 and apap1-4 mutant plants, and from wild type, were sequentially extracted with increasingly harsh solvents to release wall polymers (Zhu et al., 2010, Mol. Plant. 3:818-833) and the wall extracts were analyzed using a comprehensive array of 155 monoclonal antibodies (Pattathil et al., 2010, Plant Physiol. 153:514-525) reactive against diverse epitopes present on most major plant cell wall glycans (FIG. 12, FIG. 13; and Table 12). There was a 5- to 83-fold (for different antibodies) increase of RG-I backbone and HG epitopes in the milder solvent (oxalate, carbonate, and 1 M KOH) fractions from apap1-3 and apap1-4 aerial tissue walls compared to wild type (see the box corresponding to HG backbone-1 and RG-1 backbone, FIG. 12). Likewise, a 9- to 49-fold increase in xylan-4 through xylan-7 epitopes (see the box corresponding to xylan groups 3-7, FIG. 12) was obtained in the milder solvent (oxalate and carbonate) fractions. The two mutant lines exhibited similar changes in their glycome-profiles.

TABLE 12
List of plant cell wall glycan-directed monoclonal antibodies used
for ELISA (See FIG. 4) and glycome profiling analyses (FIGS. 12, 13,
and 14). The groupings of antibodies are based on a hierarchical clustering
of ELISA data generated from a screen of all monoclonal antibodies
against a panel of plant polysaccharide preparations (Pattathil et
al., 2010, Plant Physiol. 153: 514-525) that group the antibodies
according to the predominant polysaccharides that they recognize.
Detailed descriptions of each antibody, including immunogen, antibody
isotype, epitope structure (to the extent known), supplier information,
and related literature citations may be found at the the WallMabDB
plant cell wall monoclonal antibody database (available through the
world wide web at wallmabdb.net).

| Glycan group | | Glycan group | |
|---|---|---|---|
| recognized | mAB Names | recognized | mAB Names |
| Non-Fucosylated | CCRC-M95 | Xylan-1/XG | CCRC-M111 |
| xyloglucan-1 | CCRC-M101 | | CCRC-M108 |
| Non-Fucosylated | CCRC-M104 | | CCRC-M109 |
| xyloglucan-2 | CCRC-M-89 | Xylan-2 | CCRC-M119 |
| | CCRC-M93 | | CCRC-M115 |
| | CCRC-M87 | | CCRC-M110 |
| | CCRC-M88 | | CCRC-M105 |
| Non-Fucosylated | CCRC-M100 | Xylan-3 | CCRC-M117 |
| xyloglucan-3 | CCRC-M103 | | CCRC-M113 |
| Non-Fucosylated | CCRC-M58 | | CCRC-M120 |
| xyloglucan-4 | CCRC-M86 | | CCRC-M118 |
| | CCRC-M55 | | CCRC-M116 |
| | CCRC-M52 | | CCRC-M114 |
| | CCRC-M99 | Xylan-4 | CCRC-M154 |
| Non-Fucosylated | CCRC-M54 | | CCRC-M150 |
| xyloglucan-5 | CCRC-M48 | Xylan-5 | CCRC-M144 |
| | CCRC-M49 | | CCRC-M146 |
| | CCRC-M96 | | CCRC-M145 |
| | CCRC-M50 | | CCRC-M155 |
| | CCRC-M51 | Xylan-6 | CCRC-M153 |
| | CCRC-M53 | | CCRC-M151 |
| Non-Fucosylated | CCRC-M57 | | CCRC-M148 |
| xyloglucan-6 | | | |
| Fucosylated | CCRC-M102 | | CCRC-M140 |
| xyloglucan | CCRC-M39 | | CCRC-M139 |
| | CCRC-M106 | | CCCRC-M138 |
| | CCRC-M84 | Xylan-7 | CCRC-M160 |
| | CCRC-M1 | | CCRC-M137 |
| Galactomannan-1 | CCRC-M75 | | CCRC-M152 |
| | CCRC-M70 | | CCRC-M149 |
| | CCRC-M74 | | CCRC-M60 |
| Galactomannan-2 | CCRC-M166 | | CCRC-M41 |
| | CCRC-M168 | | CCRC-M80 |
| | CCRC-M174 | | CCRC-M79 |
| | CCRC-M175 | | CCRC-M44 |
| Acetylated mannan | CCRC-M169 | RG-I/Arabino- | CCRC-M33 |
| | CCRC-M170 | galactan | CCRC-M32 |
| β-Glucan | LAMP | | CCRC-M13 |
| | BG1 | | CCRC-M42 |
| HG Backbone-1 | CCRC-M131 | | CCRC-M24 |
| | CCRC-M38 | | CCRC-M12 |
| | JIM5 | | CCRC-M7 |
| HG-Backbone-2 | JIM136 | | CCRC-M77 |
| | JIM7 | | CCRC-M25 |
| RG-I Backbone | CCRC-M69 | | CCRC-M9 |
| | CCRC-M35 | | CCRC-M128 |
| | CCRC-M36 | | CCRC-M126 |
| | CCRC-M14 | | CCRC-M134 |
| | CCRC-M129 | | CCRC-M125 |
| | CCRC-M72 | | CCRC-M123 |
| Linseed Mucilage | JIM3 | | CCRC-M122 |
| RG-I | CCRC-M40 | | CCRC-M121 |
| | CCRC-M161 | | CCRC-M112 |
| | CCRC-M164 | | CCRC-M21 |
| Physcomitrella | CCRC-M98 | | JIM131 |
| Pectin | CCRC-M94 | | CCRC-M22 |
| RG-Ia | CCRC-M5 | | JIM132 |
| | CCRC-M2 | | JIM1 |
| RG-Ib | JIM137 | | CCRC-M15 |
| | JIM101 | | CCRC-M8 |
| | CCRC-M61 | | JIM16 |
| | CCRC-M30 | Arabino- | JIM4 |
| RG-Ic | CCRC-M23 | galactan-3 | CCRC-M31 |
| | CCRC-M17 | | JIM17 |
| | CCRC-M19 | | CCRC-M26 |
| | CCRC-M18 | | JIM15 |
| | CCRC-M56 | | JIM8 |
| | CCRC-M16 | | CCRC-M85 |
| Arabinogalactan-1 | JIM93 | | CCRC-M81 |
| | JIM94 | | MAC266 |
| | JIM11 | | PN 16.4B4 |
| | MAC204 | Arabino- | MAC207 |
| | JIM20 | galactan-4 | JIM133 |
| Arabinogalactan-2 | JIM14 | | JIM13 |
| | JIM19 | | CCRC-M92 |
| | JIM12 | | CCRC-M91 |
| | CCRC-M133 | | CCRC-M78 |
| | CCRC-M107 | Unidentified | MAC265 |
| | | | CCRC-M97 |

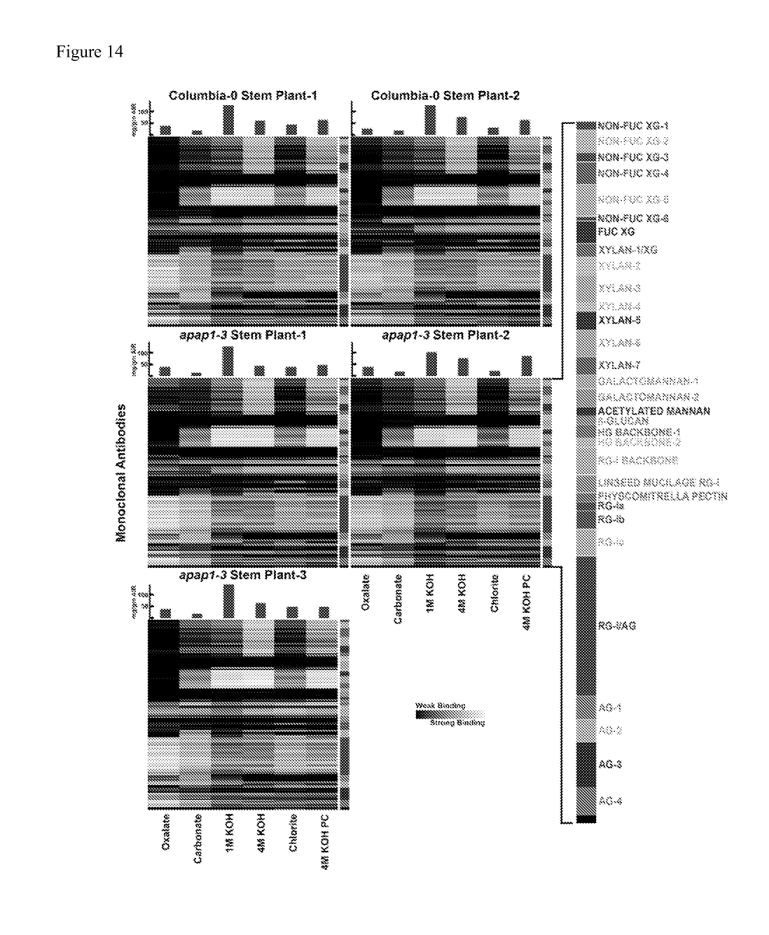

Monosaccharide composition analyses of the wall extracts confirmed the presence of significantly more Rha, GalA, and Xyl in the mild oxalate extract from apap1-3 mutant walls compared to wild type (Table 3), indicating more pectin and xylan in these fractions and supporting the glycome-profiling results. Because there was no significant difference in the total amount of material extracted from walls of wild type (269±14.1 μg) versus apap1 mutant (248.7±22.4 μg), the glycome-profiling and sugar composition analyses indicate that specific HG, RG-I and xylan fractions are more easily extracted from apap1 walls. This result supports the hypothesis that some of the pectin and xylan in the apap1 mutants is held less tightly in the wall because the mutants lack the core protein of the APAP1 structure (FIG. 9). Similar glycomeprofiling analyses of 8-week-old apap1-3 mutant and wild type stems indicated more subtle differences in the distribution of wall glycan epitopes in cell wall extracts from mutant versus wild type stems (See FIG. 14). There was a 2- to 13-fold increase in HG and RG-I backbone epitopes in the most easily extracted (oxalate) cell wall fractions from the mutant, consistent with the conclusion that a loss of the APAP1 core protein AtAGP57C results in reduced covalent linkages in at least a fraction of pectin in Arabidopsis stem cell walls. Taken together the results indicate that APAP1 is a structural proteoglycan in plant cell walls.

### Discussion

The core protein of APAP1 has recently been classified as one of 22 classical AGPs, named AGP57C, in a bioinformatics analysis (Showalter et al., 2010, Plant Physiol. 153:485-513). The mature AGP described here, after posttranslational modification, is a large proteoglycan that harbors three different glycan domains: type II AG, pectin and arabinoxylan. Among the 23 proline residues in AtAGP57C, 12 to 14 are predicted to be hydroxylated from which 8 Hyp residues are predicted to be attachment sites for AG addition (Kieliszewski, 2001, Phytochemistry 57:319-323). This matches the Hyp-O-glycoside profile of APAP1 reported in this paper in which 70% of the Hyp residues were shown to contain attached polysaccharides.

The two different glycosylation forms of APAP1, YS1 and YS2, contain similar amounts of acidic sugar residues (8.72 μg/100 μg YS1 and 9.57 μg/100 μg YS2) and thus have similar overall charges. However, they have significantly different glycan content suggesting heterogeneity in their physical and chemical properties. A comparison of the Hyp:Gal:Ara:Xyl:Rha:GalA/GlcA ratios of YS1 and YS2 indicate that on average, a given Hyp residue in AtAGP57C of YS1 has approximately 24 Gal, 45 Ara, 28 Xyl, 7 Rha and 13 GalA/GlcA while in YS2 there are on average 11 Gal, 65 Ara, 65 Xyl, 9 Rha, and 13 GalA/GlcA residues. Because linkage analyses indicated a terminal-Xyl to internal 4-Xyl/2,4-Xyl ratio above 10 for YS1 and about 6 for YS2, the results suggest that YS1 has 2 to 3 arabinoxylan oligomer chains of an average DP 11 on each Hyp-O-polysaccharide, while YS2 has 6-7 arabinoxylan/xylan oligomer chains of an average DP 7 per each Hyp-O-polysaccharide.

Because APAP1 was isolated from suspension culture media, a question that arises is whether APAP1 is a glycoconjugate generated by enzymes in the culture medium or rather is a glycoconjugate that exists in the walls of tissues in the plant? Numerous prior reports have suggested the possible existence of such complex structures in plant tissues. Among these complex structures, pectic AGP fractions have been broadly reported from different plant tissues, including seeds of Coix lacryma-jobivar (Yamada et al., 1987, Phytochemistry 26:3269-3275), Zea shoots (Kato and Nevins, 1992, Carbohydr. Res. 227:315-329), grapes (Pellerin et al., 1995, Carbohydr. Res. 277:135-143), roots of Angelica acutiloba (Zhang et al., 1996, Carbohydr. Polym. 31:149-156), spent hops (Oosterveld et al., 2002, Carbohydr. Polym. 49:407-413), roots of Vernonia kotschyana (Nergard et al., 2005, Carbohydr. Res. 340:115-130), sugar beet (McKenna et al., 2006, FFI J. 211:264-274), and carrot taproot (Immerzel et al., 2006, Physiol. Plant. 128:18-28). Also, the xylose to pectin linkage was identified in a pectic arabinogalactan polysaccharide isolated from leaves of Diospyros kaki (Duan et al., 2004, Plant Physiol., 153:403-419), and might also exist in the xylose-rich pectins purified from pea hulls (Renard et al., 1997, Int. J. Biol. Macromol. 21:155-162) and flax seed mucilage (Naran et al., 2008, Plant Physiol. 148:132-141). Furthermore, Selvendran and colleagues (Stevens and Selvendran, 1984, Phytochemistry 23:339-347; Ryden and Selvendran, 1990, Biochem. J. 269:393-402) systematically studied wall polysaccharides extracted from diverse tissues of different plants, including runner bean, mung bean, cabbage and tomato. Based on these studies, the authors suggested the presence of wall polysaccharide-protein and wall polysaccharide-polyphenol-Hyp-rich glycoprotein complexes in those plant tissues.

The challenges associated with the definitive identification of covalent linkages between the polysaccharides and proteins in the earlier studies prevented firm conclusions to be made regarding the existence of polysaccharide-protein proteoglycans in plants. However, the early studies from multiple independent labs using diverse plant tissues and plant types support the existence of APAP1-like complexes in the walls of plant tissues and hence their function and biosynthesis in plants (Tan et al., 2012, Front. Plant Sci. 3:article 140). Multiple and diverse methods and numerous controls were used in the present study to show that the pectin and arabinoxylan domains in APAP1 are covalently attached to the AGP. These results suggest that some AGPs may serve as a type of “cross-linker” in cell walls, connecting at least some of the pectin and hemicellulose polysaccharides with at least one AGP, thereby providing the possibility of forming a continuous network between wall polysaccharides and wall structural proteins. This type of the plant cell wall model lies between that proposed by Keegstra et al. (1973, Plant Physiol. 51:188-197) and the more current “tethered network model” (Park and Cosgrove, 2012, Plant Physiol. 158:465-475).

Why can APAP1 be secreted into the culture media and why is APAP1 present at such a low concentration in the culture medium compared to normal AGPs? As we calculated above, a Hyp residue in APAP1 that bears a polysaccharide chain has an average of 13 GalA/GlcA residues and multiple xylan chains with an average length of 7-11 xylosyl residues. This suggests that both the pectin and xylan domains of APAP1 are much shorter than those of native pectins (minimum of 70-100 GalA residues for homogalacturonan regions) (Thibault et al., 1993, Carbohydr. Res., 238:271-286) and xylans (up to 500 kDa) (Porchia and Scheller, 2000, Physiol. Plant. 110:350-356). We hypothesize that APAP1 with longer pectin and arabinoxylan chains may exist in the cell walls and that such macromolecules are highly cross-linked and interwoven in the walls, and thus, cannot be released into the culture media. However, further research on such structures is needed to test this.

The location of APAP1 synthesis is yet to be determined. Pectin and xylan synthesis occur in the Golgi (Mohnen, 2008, Curr. Opin. Plant Biol., 11:266-277; Brown et al., 2011, Plant J., 66:401-413) and AGP synthesis initiates in the ER and continues in the Golgi (Oka et al., 2010, Plant Physiol. 152:332-340; Wu et al., 2010, J. Biol. Chem. 285:13638-13645). Thus, one possible mechanism for APAP1 biosynthesis is the addition of glycans onto AtAGP57C by either the consecutive addition of one residue at a time or by block addition, possibly occurring within the Golgi apparatus (Atmodjo et al., 2011, Proc. Natl. Acad. Sci. USA 108:20225-20230). This mechanism would require that the AGP, pectin and hemicellulose biosynthetic enzymes work in a spatially and temporally coordinated fashion to synthesize such proteoglycans. Alternatively, pectin and arabinoxylan glycans may become attached to AGP in the extra-cellular matrix by endotransglycosylases, in a manner comparable to xyloglucan restructuring in the wall (Fry et al., 1992, Biochem. J., 282:821-828; Rose et al., 2002, Plant and Cell Physiol. 43:1421-1435).

It has recently been proposed that some noncellulosic polysaccharides, like (1,3; 1,4)-β-glucans may be synthesized via a two-phase mechanism where oligosaccharides are synthesized in the Golgi apparatus as oligosaccharides attached to a recyclable lipid or protein, transported via vesicles to the wall, and then transferred to a growing polymer chain via a second stage of synthesis at the plasma membrane (Burton et al., 2010, Nature Chem. Biol., 6:724-732). Evidence to support this model is wanting. However, the transfer of pectin and arabinoxylan glycans from glyco-lipid intermediates onto AGP in the Golgi or to GPI-anchored AGP on the plasma membrane, followed by subsequent cleavage of the GPI-anchor and deposition into the cell wall, are possible mechanisms for the synthesis of APAP1-like molecules. The results presented here do not indicate how many diverse AGPs act as core(s)/acceptor(s) for the synthesis of diverse proteoglycan structures, nor do they make clear how much structural variation there is within diverse APAP1-like structures.

AGPs, pectin and to a lesser extent arabinoxylans exist in a spectrum of structural forms in cell walls, culture media, and plant extracellular exudates such as gums. Their multiple functions include providing structural support to the wall and serving as carbohydrate reserves, signaling molecules, cell-cell adhesion factors, and water retention polymers (Ellis, et al., 2010, Plant Physiol. 153:403-419). The identification of the Arabinoxylan-Pectin-Arabinogalactan Protein 1, APAP1, suggests that at least one AGP serves as a cross-linker for at least a subfraction of pectin and arabinoxylan. The existence of APAP1 in plant walls has significant consequences for our understanding of wall architecture and function, and potentially for engineering plant cell walls for improved agronomic and renewable biomaterial uses.

### Example 2

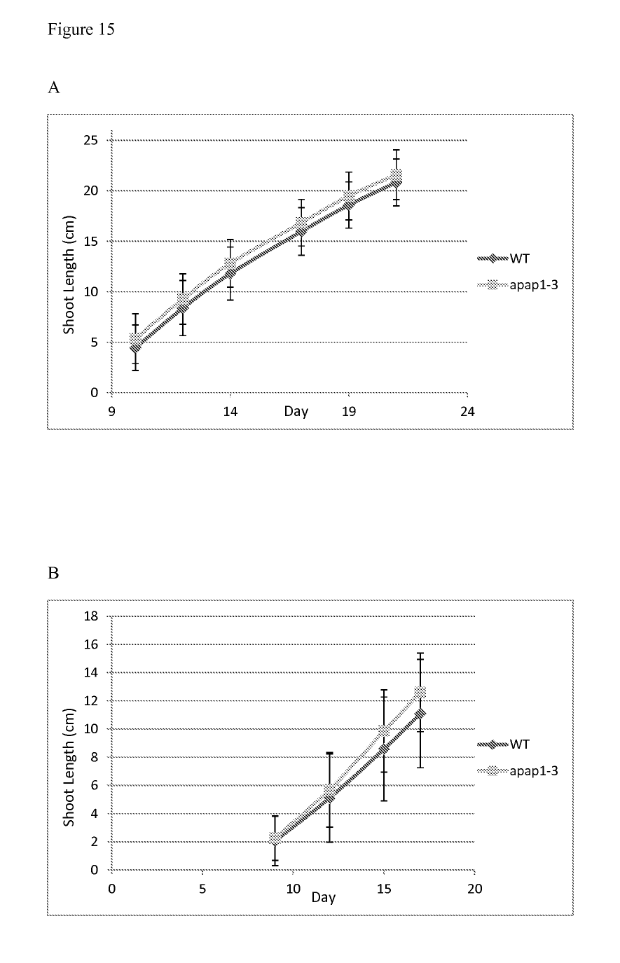

apap1-3 transgenic Arabidopsis and wild-type Arabidopsis were grown in a growth chamber under standard conditions. At days 10, 12, 14, 17, 19, and 21 the shoot length was measured. The apap1-3 mutant plants had a longer shoot length than the wild-type plants, and the difference was significant at days 10, 12, 14, 17, and 19 (see FIGS. 15a and 15b). The increased shoot length of the transgenic plants indicates they have increased growth and shoot elongation compared to the wild-type plants.

### Example 3

apap1-3 transgenic Arabidopsis and wild-type Arabidopsis were grown in a growth chamber under standard conditions. The release of glucose and xylose from the 8 week old plants was evaluated by a combined high-throughput pretreatment and enzymatic hydrolysis process (Studer et al., 2010, Biotechnol. Bioeng., 105:231-238) and release of xylose was found to be significantly higher in apap1-3 transgenic Arabidopsis plants (see Table 13). The measurement of cell wall sugars (glycosyl residue composition by TMS derivatization and GC/MS as described in example 1) revealed that apap1-3 had increase total sugar compared to wild type Table 14). The increased release of xylose and increased total sugars indicates transgenic plants having decreased expression of an AGP polypeptide core have decreased recalcitrance and increased total sugar compared to wild-type plants.

TABLE 13
Sugar release data of alcohol insoluble residues (AIR) of cell
walls (biomass) isolated from aerial tissues of apap1 mutant vs.
wild type Arabidopsis plants. For glucose release, although
the average glucose amount from apap1 mutant AIR is higher than
that of WT AIR, there is no significant difference between mutant
and WT (T-test, p = 0.58371 > 0.05). However, released
xylose amount from apap1 mutant AIR is significantly higher
than that from WT AIR (T-test, p = 0.01588 < 0.05).

| | Glucose (g/g biomass) | Xylose (g/g biomass) |
|---|---|---|
| WT | 0.16731 ± 0.02085 | 0.07613 ± 0.00831 |
| apap1-3 | 0.17047 ± 0.01719 | 0.08248 ± 0.00894 |

TABLE 14
Sugar composition analysis of different cell wall extracts from apap1-3 aerial tissues vs. WT aerial
tissues. Three biological samples (plants) of mutant and two WT biological samples (plants) were
analyzed and the data were averaged, respectively. Pairs in bold are significantly different from
each other. Data are microgram/300 microgram cell wall extract^{a} or microgram/2.1 mg cell wall extract^{b}.

| Sample | | Ara | Xyl | Fuc | Rha | Glc |
|---|---|---|---|---|---|---|
| Oxalate^{a} | Apap1-3 | 18.05 ± 6.54 | 3.08 ± 0.18 | 1.67 ± 0.20 | 20.21 ± 2.89 | 3.59 ± 2.91 |
| | WT | 10.13 ± 8.49 | 2.27 ± 0.32 | 1.45 ± 0.76 | 8.69 ± 2.04 | 1.85 ± 0.02 |
| Carbo- | Apap1-3 | 28.22 ± 11.22 | 14.75 ± 11.14 | 1.63 ± 1.22 | 18.90 ± 3.39 | 16.69 ± 20.39 |
| nate^{a} | WT | 12.93 ± 9.51 | 3.79 ± 3.31 | 0.74 ± 0.17 | 11.81 ± 5.62 | 3.52 ± 0.28 |
| 1M | Apap1-3 | 7.17 ± 5.77 | 65.48 ± 8.57 | 0.22 ± 0.20 | 4.59 ± 1.57 | 18.49 ± 12.57 |
| KOH^{a} | WT | 6.78 ± 5.03 | 71.82 ± 30.94 | 0.35 ± 0.49 | 2.32 ± 0.47 | 33.12 ± 29.41 |
| 4M | Apap1-3 | 8.96 ± 3.11 | 109.43 ± 51.84 | 3.13 ± 1.65 | 6.10 ± 2.28 | 25.53 ± 11.80 |
| KOH^{a} | WT | 5.25 ± 4.77 | 60.58 ± 20.61 | 1.81 ± 1.18 | 3.80 ± 2.31 | 18.12 ± 7.37 |
| Chlorite^{a} | Apap1-3 | 27.76 ± 11.17 | 6.60 ± 3.22 | 1.49 ± 1.79 | 12.90 ± 7.66 | 49.28 ± 25.34 |
| | WT | 16.06 | 1.56 | 1.56 | 7.70 | 10.52 |
| PC 4M | Apap1-3 | 20.42 ± 10.37 | 116.80 ± 42.63 | 2.98 ± 1.07 | 14.82 ± 9.04 | 24.98 ± 10.43 |
| KOH^{a} | WT | 5.45 ± 6.38 | 27.48 ± 19.58 | 0.76 ± 1.07 | 4.46 ± 4.02 | 7.50 ± 5.19 |
| Left | Apap1-3 | 0.53 ± 0.50 | 1.63 ± 1.91 | 0 | 0.81 ± 0.84 | 46.55 ± 4.95 |
| residues^{a} | WT | 0 | 0.84 ± 0.94 | 0 | 0.09 ± 0.13 | 18.87 ± 9.82 |
| Total^{b} | Apap1-3 | 111.11 ± 28.81 | 317.76 ± 82.81 | 11.13 ± 3.36 | 78.33 ± 20.24 | 185.10 ± 47.28 |
| | WT | 48.56 ± 45.07 | 168.32 ± 114.25 | 6.66 ± 3.96 | 38.86 ± 28.30 | 93.49 ± 68.53 |

| Sample | | Gal | Man | GlcA | GalA |
|---|---|---|---|---|---|
| Oxalate^{a} | Apap1-3 | 18.15 ± 7.38 | 2.05 ± 1.31 | 17.85 ± 7.44 | 135.39 ± 41.27 |
| | WT | 10.84 ± 7.54 | 0.76 ± 0.08 | 28.47 ± 8.16 | 43.56 ± 12.47 |
| Carbo- | Apap1-3 | 18.81 ± 6.47 | 2.41 ± 1.22 | 8.99 ± 5.88 | 54.42 ± 6.70 |
| nate^{a} | WT | 13.58 ± 7.35 | 1.64 ± 1.58 | 10.35 ± 6.25 | 17.40 ± 15.43 |
| 1M | Apap1-3 | 4.01 ± 1.71 | 1.13 ± 0.67 | 3.54 ± 2.87 | 7.04 ± 3.16 |
| KOH^{a} | WT | 5.76 ± 3.22 | 0.59 ± 0.02 | 5.70 ± 0.16 | 6.91 ± 3.79 |
| 4M | Apap1-3 | 12.67 ± 5.98 | 5.22 ± 2.25 | 7.58 ± 3.18 | 12.43 ± 7.30 |
| KOH^{a} | WT | 10.96 ± 7.84 | 6.84 ± 8.61 | 4.82 ± 2.35 | 10.95 ± 6.32 |
| Chlorite^{a} | Apap1-3 | 11.74 ± 2.55 | 1.12 ± 0.40 | 6.86 ± 7.22 | 22.21 ± 12.02 |
| | WT | 13.64 | 0.36 | 2.34 | 18.76 |
| PC 4M | Apap1-3 | 12.16 ± 3.01 | 3.83 ± 1.25 | 5.86 ± 6.20 | 29.03 ± 5.93 |
| KOH^{a} | WT | 5.23 ± 4.95 | 3.23 ± 3.78 | 4.27 ± 0.10 | 11.18 ± 8.02 |
| Left | Apap1-3 | 2.43 ± 2.16 | 1.63 ± 2.23 | 0 | 1.77 ± 3.07 |
| residues^{a} | WT | 0.92 ± 1.29 | 2.83 ± 0.40 | 0 | 0 |
| Total^{b} | Apap1-3 | 79.98 ± 8.39 | 17.39 ± 6.07 | 50.68 ± 21.95 | 262.29 ± 57.30 |
| | WT | 60.92 ± 29.75 | 16.23 ± 7.79 | 55.95 ± 17.41 | 108.76 ± 100.33 |

### Example 4

### Pollen-Specific Leucine-Rich Repeat Extensin-Like Protein 1 (PLRX1_ARATH) (AEE76184)

### Objective

This goal of this work was to isolate and characterize any APAP1-like proteoglycan complex, focusing on glycoproteins covalently attached with xyloglucan, a major hemicellulose in the plant cell wall.

### Methods

### 1. Isolation of A104P, a Xyloglucan Containing Extensin.

Arabidopsis suspension culture media were dialyzed and loaded onto a strong anion exchange column (Q-sepharose) as described in Example 1. The material that voided on the column (neutral fraction) was collected, dialyzed against water, and freeze-dried. The dried residues were named as A104.

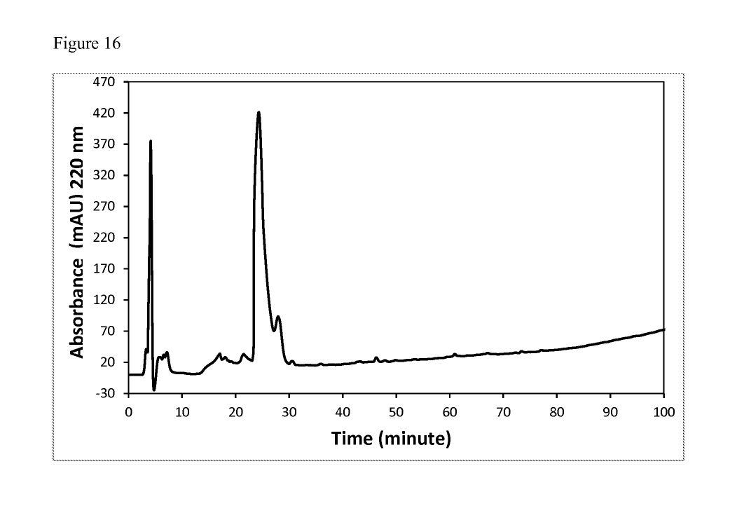

A104 was further separated on a size exclusion column (Superpose 12 column), followed by purification on a reverse phase column (PRP-1 column) as shown in Example 1. The fraction at 27 min on the reverse phase column (FIG. 16) was collected and lyophilized, and named as A104P.

### 2. Sugar Composition and Linkage Analysis

See methods in Example 1.

### 3. Deglycosylation of A104P and Tryptic Peptide Isolation of Deglycosylated A104P

Methods of deglycosylation were described in Example 1. The deglycosylated A104P was digested with trypsin, and the tryptic peptides were sequenced by LC-MS/MS.

4. MALDI-TOF MS Analysis of Hyp-O-Glycans Generated from A104P

Hyp-O-glycans were prepared by base hydrolysis as described in example 1, and analyzed by MALDI-TOF MS.

### 5. Mutant Characterization

Seeds of 5 insertion mutant lines of gene At3g19020 were ordered from ABRC. The seeds were germinated and planted in a growth chamber as described in Example 1. The plants were genotyped with primers as listed in Table 15.

TABLE 15
SALK lines bearing insertion mutation of gene
At3g19020 were screened for homozygotes by
using primers as listed. All mutant lines have
insertion at different exon regions.

| | | LP primer | RP primer |
|---|---|---|---|
| Mutant | Salk line | (SEQ ID NO:) | (SEQ ID NO:) |
| XG-1 | SALK_031536C | TACCAAAAACCA | AGAACACACGTCT |
| | | AGCTCCATG (38) | GGACCAAC (39) |
| XG-2 | SALK_070551 | GGACGGTAGTAT | GACTTTTCGGAAT |
| | | CGGCTGATC (40) | AACTCCGC (41) |
| XG-3 | SALK_074498 | GGACGGTAGTAT | GACTTTTCGGAAT |
| | | CGGCTGATC (42) | AACTCCGC (43) |
| XG-4 | SALK_001365 | AGGCTTAGGTGA | TTGGTAAATCAAC |
| | | CTCCTCTGG (44) | AGCTTCCG (45) |
| XG-5 | SALK_132358 | AAAGAGTTCAAA | TGATTTACCAATT |
| | | ACGCAAACG (46) | GTCTCGGG (47) |

### Results

1. Gene Identified from A104P

The pollen-specific leucine-rich repeat extensin-like protein 1, At3g19020, was identified (see FIG. 1, SEQ ID NO:14).

### 2. Sugar Composition and Linkage Analysis

TABLE 16
Sugar composition of A104P. TMS method was used in this
analysis. Data are presented in mol %. A104P contained
only neutral sugar residues, different from APAP1.

| Glycosyl Residues | Glycosyl Composition mol % |
|---|---|
| Xyl | 34.4 |
| Glc | 49.3 |
| Gal | 16.3 |

Sugar accounts for 33.66% of sample dry weight.

TABLE 17
Glycosyl linkage analysis of A104P. The significant amount
of 4-Glc and 4,6-Glc as well as t-Xyl and 2-Xyl demonstrated
that the carbohydrate domain of A104P was xyloglucan.

| Glycosyl Linkage | Mol % |
|---|---|
| Terminally linked Fucopyranosyl residue (t-Fuc) | 1.1 |
| Terminally linked Xylopyranosyl residue (t-Xyl) | 15.3 |
| Terminally linked Glucopyranosyl residue (t-Glc) | 0.5 |
| Terminally linked Galactopyranosyl residue (t-Gal) | 9.2 |
| 4-linked Arabinopyranosyl residue or 5-linked | 0.1 |
| Arabinofuranosyl residue (4-Arap or 5-Araf) | |
| 2-linked Xylopyranosyl residue (2-Xyl) | 5.0 |
| 4-linked Mannopyranosyl residue (4-Man) | 0.2 |
| 2-linked Galactopyranosyl residue & 6 linked | 3.8 |
| Glucopyranosyl residue (2-Gal + 6-Glc) | |
| 4-linked Glucopyranosyl residue (4-Glc) | 25.4 |
| 4,6-linked Mannopyranosyl residue (4,6-Man) | 0.2 |
| 4,6-linked Glucopyranosyl residue (4,6-Glc) | 39.1 |
| 3,4,6-linked Glucopyranosyl residue (3,4,6-Glc) | 0.2 |

3. MALDI-TOF MS Analysis Data of Hyp-O-Glycans Isolated from A104P.

TABLE 18
Hyp-glycan ions identified via MALDI-TOF MS. The different ions
plus the above glycosyl linkage analysis supported the direct
O-linkage of xyloglucosyl oligosaccharides to hydroxyproline,
a novel linkage in the plant cell wall. Further MS/MS analysis
will be carried out to make certain that the Hex to Hex linkages
are 1 to 4, and the hexose residues are Glc.

| Ions (+Na⁺) | m/z |
|---|---|
| Hyp-O-Hex | 316 |
| Hyp-O-Hex5 | 964 |
| Hyp-O-Hex5Pen1 | 1096 |
| Hyp-O-Hex4Pen2 | 1066 |
| Hyp-O-Hex4Pen3 | 1198 |
| Hyp-O-Hex3-Pen2 | 904 |

### 4. Mutant Analysis of A104P.

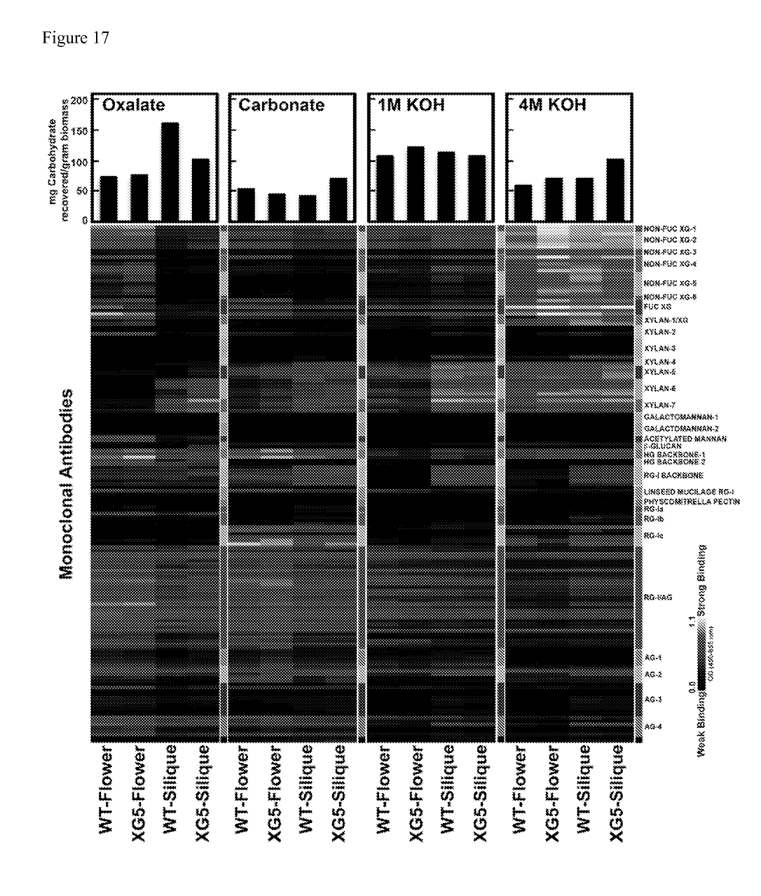

Homozygote line XG-5, with insertion mutation at the exon region, was characterized. Whole aerial tissue, siliques, and flowers were collected separately from the XG-5 mutants. Alcohol insoluble residues were prepared from above different tissues, followed by extracted with increasing harsh solvent, 50 mM ammonium oxalate, 50 mM sodium carbonate, 1 M KOH, and 4 M KOH. The different extracts were probed with the Plant Cell Wall Monoclonal Antibody Kit (Pattathil et al., 2010, Plant Physiol. 153:514-525). The results show that there is no significant difference between the extracts of whole aerial tissues from XG-5 mutants and WT plants. However, the 4 M KOH extract of XG-5 flowers exhibits significantly higher binding to monoclonal antibodies raised against different xyloglucans (FIG. 17). This result suggests that the absence of PEX1 in Arabidopsis flowers lead to either easier extraction of xyloglucan from the mutant flowers than from the WT or more xyloglucans accumulated in the mutant flowers. More subtle binding difference is also showed between the extracts from siliques of XG-5 mutant and WT plants.

### Conclusions:

1. Based on the glycosyl composition and linkage analyses, the carbohydrate domain of A104P was typical xyloglucan, and the protein backbone of A104 was the pollen-specific leucine-rich repeat extensin-like protein 1 corresponding to gene At3g19020.

2. MALDI-TOF MS identified a few ions corresponding to Hyp-O-Hexₙ, supporting the direct linkage between Hyp and Hexose. With aid of glycosyl linkage data, the ions was determined as Hyp-O-Glcₙ, a novel attachment between Hyp of extensin and xyloglucan. Therefore A104P is a xyloglucan-extensin complex.

3. One mutant line, XG5, was characterized as homozygote. The seeds of this line showed a significant early germination and long root length phenotype. Further characterization of other phenotype is on going.

### Example 5

To search for APAP1-like proteoglycans in Arabidopsis stems that may relate to stem growth and to quantify such proteoglycans in stems, glycoproteins were purified from the different extracts of alcohol insoluble residues isolated from WT Arabidopsis stems. Glycosyl composition analysis was carried out to determine what type of glycoproteins exist in each extract. Tryptic peptides generated from deglycosylated stem glycoproteins were analyzed for APAP1-like proteoglycans.

TABLE 20
Glycoprotein recovery from extracts of AIR (alcohol insoluble residues) of WT Arabidopsis
stems. Wild type Arabidopsis stems from individual plants were harvested at 6 weeks
of age. AIR samples were prepared from those stems as described in the methods. Different
extractions were carried out in the order of oxalate, carbonate, 1M KOH, 4M KOH, chlorite,
and post chlorite 4M KOH. Two parallel trials were tested. Glycoproteins (GP) were purified
from each extract as the method of purification of APAP1, and the yields of glycoproteins
from total soluble extracts were calculated. The percentage of glycoproteins in total soluble
material in each extract was comparable between the two parallel trials. Yariv perceptible
GPs of each extract were measured by Yariv precipitation assay. Hyp content of GPs from
each extract were measured colorimetrically as described in the method.

| | Experiment 1 from 324 mg AIR | | | | Experiment 2 from 287 mg AIR | | | | | |
|---|---|---|---|---|---|---|---|---|---|---|
| | | Glyco- | GP/ | GP/ | | Glyco- | GP/ | GP/ | Yariv | Hyp/ |
| | | protein | soluble | AIR | | protein | soluble | AIR | Precipitate | GP |
| | Soluble | (GP) | % | % | Soluble | (GP) | % | % | of GP/GP | % |
| Sample | (mg) | (mg) | (w/w) | (w/w) | (mg) | (mg) | (w/w) | (w/w) | % (w/w) | (w/w) |
| Oxalate | 8.70 | 2.55 | 29.3 | 0.787 | 6.17 | 1.59 | 25.8 | 0.554 | 19.64 | 1.13 |
| Carbonate | 4.41 | 0.77 | 17.5 | 0.238 | 2.52 | 0.33 | 13.1 | 0.115 | 12.14 | 0.80 |
| 1M KOH | 21.16 | 3.64 | 17.2 | 1.123 | 16.24 | 3.08 | 19.0 | 1.073 | 1.20 | 0.60 |
| 4M KOH | 5.84 | 4.09 | 70.0 | 1.262 | 3.78 | 2.74 | 72.5 | 0.955 | 0 | 0.46 |
| Chlorite | 10.47 | 1.82 | 17.4 | 0.562 | 6.47 | 0.86 | 13.3 | 0.300 | 7.74 | 0.67 |
| PC 4M | 6.38 | 2.20 | 34.5 | 0.679 | 4.80 | 1.73 | 36.0 | 0.603 | 0 | 0.80 |
| KOH | | | | | | | | | | |
| Total | 56.96 | 15.07 | 26.5 | 4.651 | 39.98 | 10.33 | 25.8 | 3.600 | 6.79 | 0.74 |

TABLE 21
Sugar composition analysis of glycoproteins (GP) isolated from each extract of WT
Arabidopsis stem. Data are presented as μg/100 μg of GP. TMS method was
used in this analysis. The oxalate and carbonate extractions released most pectins.

| | | | | | | | | | | Sugar % |
|---|---|---|---|---|---|---|---|---|---|---|
| | | | | | | | | | | (w/w |
| Sample | Ara | Xyl | Fuc | Rha | Gal | Glc | Man | GalA | GlcA | of GP) |
| GP- | 8.7 | 0 | 0 | 3.6 | 12.2 | 0 | 0 | 36.6 | 3.7 | 64.8 |
| Oxalate | | | | | | | | | | |
| GP- | 6.6 | 13.9 | 0 | 2.6 | 5.5 | 0 | 0 | 17.6 | 4.5 | 50.7 |
| Carbonate | | | | | | | | | | |
| GP-1M KOH | 1.6 | 88.3 | 0 | 1.1 | 3.0 | 0 | 0 | trace | Trace | 94.0 |
| GP-4M KOH | 1.3 | 48.2 | 1.2 | 0.8 | 7.7 | 8.4 | 9.9 | Trace | trace | 77.5 |
| GP- | 6.8 | 11.2 | 0 | 4.0 | 8.2 | 8.3 | trace | 5.7 | trace | 44.2 |
| Chlorite | | | | | | | | | | |
| GP-4M | trace | 58.7 | 0 | 1.1 | 4.6 | 3.9 | 7.1 | 0 | trace | 75.4 |
| KOH PC | | | | | | | | | | |

One AGP, AtAGP18 (At4g37450, SEQ ID NO:8), was identified from the oxalate extract of 6-week-old wild-type Arabidopsis stems by LC-MS/MS. AtAGP18 is a APAP1-like pectic AGP as it contains large amount of GalA residues.

### Conclusions:

1. The weight percentage of APAP1-like proteoglycans in the soluble materials of each extract showed that these proteoglycans account for 26-29%, 13-17%, 17-19%, 70-72%, and 34-36% in the oxalate, carbonate, 1M KOH, 4M KOH, 4M KOH after chlorite treatment, respectively. This measurement suggests that APAP1-like proteoglycans are major component of Arabidopsis plant tissues.

2. Glycosyl composition analysis suggests that the proteoglycans in the oxalate extract are pectic-AGPs, in the carbonate extract are arabinoxylan-pectin-AGP (APAP), in the 1 M KOH, 4 M KOH, and the 4 M KOH after chlorite treatment are mainly xylan-AGPs.

3. LC-MS sequencing of the tryptic peptides of these proteoglycans shows that AtAGP18 exists as an APAP1-like pectin-AGP form in Arabidopsis stems, at least in the oxalate extract of Arabidopsis stems.

### Example 6

### Overexpression of APAP 1

### Methods:

Construction of pBI-CaMV S35-AtAGP57C Vector

The AtAGP57C/APAP1 gene was cloned from total RNA isolated from rosette leaves of WT Arabidopsis plants using the following primers and RT-PCR. APAP1-Sense Primer with a BamHI restriction site: AA ATT GGA TCC ATG AAG CTC GAA TTC ATT ATT GTT GC (SEQ ID NO:48);

APAP1-Antisense Primer with a Sad restriction site: AA ATT GAG CTC TTA CAG AAT CTC TCT GGC GGC GTA ACC (SEQ ID NO:49).

The introduced BamHI/SacI restriction sites that flanked the cloned AtAGP57C/APAP1 gene allowed the replacement of the SS^{tob}-AP₅₁EGFP gene in the pBII21-SS^{tob}-AP₅₁EGFP vector (Tan et al, 2003, Plant Physiology, 132:1362-1369) by the AtAGP57C/APAP1 gene. The AtAGP57C/APAP1 gene was driven by a CaMV S35 promoter for overexpression in plants. The gene was sequenced using a home-designed pBI primer, CCT TCG CAA GAC CCT TCC TC (SEQ ID NO:50).

Transformation of Arabidopsis thaliana Plants

The AtAGP57C/APAP1 gene was transformed into Arabidopsis thaliana plants via the floral dip method. The constructed pBI vector was transformed into Agrobacterium via the standard freeze-thaw heat-shock method. The transformed Agrobacteria were used to infect two-week-old Arabidopsis plants via the floral dip method as described previously. The APAP1/AtAGP57C T1 seeds were collected from infected mature Arabidopsis plants.

### Selection and Analysis of the APAP1/AtAGP57C T1 Overexpression Plants

The APAP1/AtAGP57C T1 overexpression seeds were germinated and grown on ½ MS plates via Kanamycin selection. The Kanamycin-selected seedlings were planted on soil and grown in day-night cycle conditions in a growth chamber. The rosette leaf samples were harvested from 1-month-old plants, total RNA was prepared from each sample, and cDNA was synthesized. Semi-quantitative PCR was performed to compare the stem length of individual APAP1-OE plants with their APAP1/AtAGP57C-transcript level.

### Results

Individual APAP1/AtAGP57C Overexpression T1 Plants have a Stem Length Phenotype.

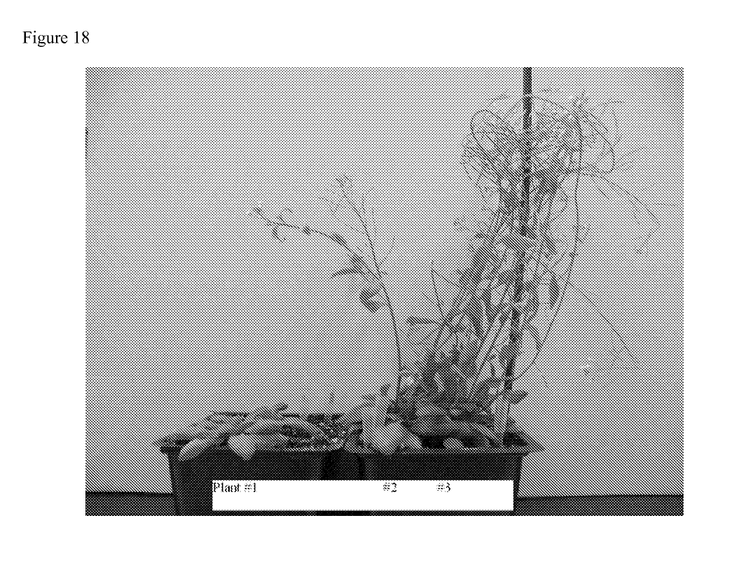

After growth on soil for one month, some T1 plants did not bolt at all, while other T1 plants had stem numbers and lengths comparable to WT Arabidopsis plants (see FIG. 18).

The stem length of APAP1/AtAGP57C overexpression T1 plants correlates to the transcript levels in APAP1/AtAGP57C overexpression transgenic plants.

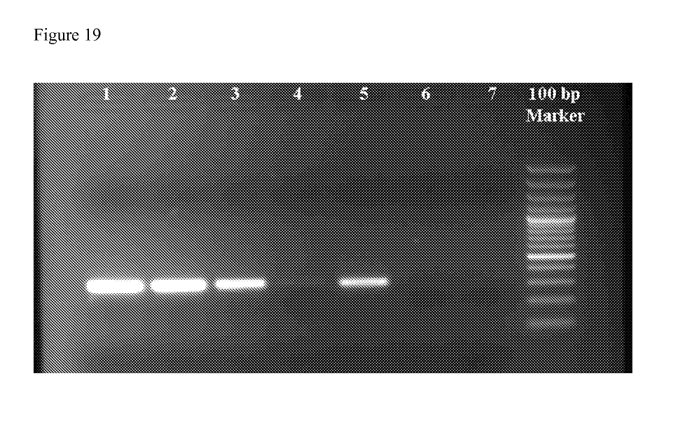

Semi-quantitative RT-PCR analysis of the plants shown in FIG. 18 revealed that the APAP1/AtAGP57C overexpression T1 plants with no stem contained the highest APAP1/AtAGP57C RNA content in their rosette leaves, while the WT-like APAP1/AtAGP57C overexpression T1 plants had low transcript levels comparable to WT levels of APAP1/AtAGP57C transcript (see FIG. 19).

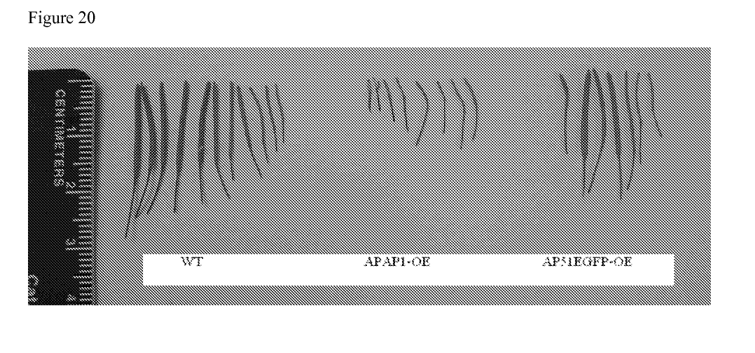

The APAP1/AtAGP57C overexpression T1 plants had a semi-sterile phenotype. We were able to harvest, depending on the severity of the overexpression phenotype, no seeds per plant, 0.1 ml seeds per plant and up to 0.5 ml of seeds per plant with the latter phenotype being found in least severe APAP1/AtAGP57C overexpression T1 plants. WT plants grown under the same growth conditions yielded 0.5 ml of seeds per plant in average (see FIG. 20).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1-6. (canceled)

7. A method for producing a metabolic product comprising:
contacting under conditions suitable for the production of a metabolic product a microbe with a composition comprising a pulp obtained from a part of a transgenic plant, wherein the transgenic plant comprises decreased expression of a coding region encoding a hydroxyproline-rich glycoprotein compared to a control plant.

8. The method of claim 7 further comprising contacting the pulp with an ethanologenic microbe.

9. The method of claim 8 wherein the ethanologenic microbe is a eukaryote.

10. The method of claim 7 further comprising obtaining a metabolic product.

11. The method of claim 10 wherein the metabolic product comprises ethanol.

12. The method of claim 7 wherein the hydroxyproline-rich glycoprotein is an arabinogalactan-protein, a leucine-rich repeat extensin-like polypeptide, a proline-rich polypeptide, an extensin-like polypeptide, or a formin-like polypeptide.

13. The method of claim 7 wherein the transgenic plant is a dicot plant.

14. The method of claim 7 wherein the transgenic plant is a monocot plant.

15. The method of claim 7 wherein the transgenic plant is a woody plant.

16. The method of claim 15 wherein the transgenic plant is a member of the genus Populus.

17. (canceled)

18. A transgenic plant comprising altered expression of a coding region encoding a hydroxyproline-rich glycoprotein compared to a control plant, wherein the transgenic plant is not Arabidopsis thaliana.

19. The transgenic plant of claim 18 wherein the hydroxyproline-rich glycoprotein is an arabinogalactan-protein, a leucine-rich repeat extensin-like polypeptide, a proline-rich polypeptide, an extensin-like polypeptide, or a formin-like polypeptide.

20. The transgenic plant of claim 18 wherein the altered expression is a decrease in expression, and wherein the transgenic plant comprises a phenotype selected from decreased recalcitrance, increased growth, or the combination thereof.

21. The transgenic plant of claim 18 wherein the transgenic plant is a dicot plant.

22. The transgenic plant of claim 18 wherein the transgenic plant is a monocot plant.

23. (canceled)

24. A part of the transgenic plant of claim 18 wherein the part is chosen from a leaf, a stem, a flower, an ovary, a fruit, a seed, and a callus.

25. The progeny of the transgenic plant of claim 18.

26. The progeny of claim 25 wherein said progeny is a hybrid plant.

27. A wood obtained from the transgenic plant of claim 18.

28. A wood pulp obtained from the transgenic plant of claim 18.

29-42. (canceled)

43. The transgenic plant of claim 18 wherein the altered expression is an increase in expression, and wherein the transgenic plant comprises a phenotype selected from shorter stems, smaller leaves, smaller inflorescences, smaller pollen, shorter roots, reduced total biomass, or a combination thereof.

44-57. (canceled)
